(19) United States
(12) Patent Application Publication
(10) Pub. No.: US 2020/0338233 A1
(43) Pub. Date: Oct. 29, 2020

(54) METHODS, POLYMER-CONTAINING FORMULATIONS, AND POLYMER COMPOSITIONS FOR TREATING RETINAL DETACHMENT AND OTHER OCULAR DISORDERS

(71) Applicant: Pykus Therapeutics, Inc., Cambridge, MA (US)

(72) Inventors: Laurence A. ROTH, Windham, NH (US); James Anthony STEFATER, III, Boston, MA (US); Tomasz Pawel STRYJEWSKI, Boston, MA (US); John Solomon GARNER, West Lafayette, IN (US)

(73) Assignee: Pykus Therapeutics, Inc., Cambridge, MA (US)

(21) Appl. No.: 16/961,496

(22) PCT Filed: Jan. 11, 2019
(86) PCT No.: PCT/US19/13185

§ 371 Date: Jul. 10, 2020

Related U.S. Application Data
(60) Provisional application No. 62/616,610, filed on Jan. 12, 2018.
(60) Provisional application No. 62/616,614, filed on Jan. 12, 2018.

(51) Int. Cl. A61L 27/26; A61L 27/58; A61L 27/52; C08L 29/04; C08L 71/02

(52) U.S. Cl. CPC A61L 27/26; A61L 27/58; A61L 27/52; C08L 29/04; C08L 71/02; A61L 2400/18; C08L 2205/025; C08L 2205/03; A61L 2400/06; A61L 2430/16; C08L 2203/02

(57) ABSTRACT

Provided are methods, polymer-containing formulations, and polymer compositions for treating retinal detachment and other ocular disorders, where the methods employ polymer compositions or polymer-containing formulations that can form a hydrogel in the eye of a subject. In certain embodiments, the hydrogel is formed by reaction of (a) a nucleo-functional polymer is a biocompatible polyalkylene polymer substituted by (i) a plurality of —OH groups, (ii) a plurality of thio-functional groups —R¹—SH wherein R¹ is an ester-containing linker, and (iii) optionally one or more —OC(O)—(C₁-C₆ alkyl) groups, such as a thiolated poly(vinyl alcohol) polymer and (ii) an electro-functional polymer that is a biocompatible polymer containing at least one thiol-reactive group, such as a poly(ethylene glycol) polymer containing alpha-beta unsaturated ester groups. In certain embodiments, the hydrogel is formed by curing a biocompatible polymer described herein, such as a thermosensitive polymer, nucleo-functional polymer, electro-functional polymer, or pH-sensitive polymer.

### CROSS-REFERENCE TO EARLIER FILED APPLICATIONS

This application is a national stage application of International Application No. PCT/US2019/013185 entitled “Methods, Polymer-Containing Formulations, And Polymer Compositions For Treating Retinal Detachment And Other Ocular Disorders,” filed on Jan. 11, 2019, which claims benefit to U.S. provisional application No. 62/616,610, filed Jan. 12, 2018, and U.S. provisional application No. 62/616,614, filed Jan. 12, 2018, each of which is incorporated by reference herein in its entirety.

### FIELD OF THE INVENTION

Methods and polymer-containing formulations or polymer compositions for treating retinal detachment and other ocular disorders, where the methods employ polymer compositions that can form a hydrogel in the eye of a subject, are provided. Also provided are ocular formulations containing a polymer composition that can form a hydrogel in the eye of a subject.

### BACKGROUND

Retinal disorders such as retinal detachments, retinal tears, and macular holes are a significant cause of vision loss in subjects. Retinal detachment is characterized by sensory layers of the retina that have become separated from their underlying supporting tissue of retinal pigment epithelium and the choroid. In many instances, retinal detachment is caused by a retinal tear or the presence of vitreous traction, either of which may occur spontaneously or may be due to trauma. Retinal detachment may also result from pathology, such as retinopathy of prematurity in premature infants or diabetic retinopathy in diabetic individuals. With time, retinal detachment can result in loss of vision, due to loss of photoreceptor cells located in the outer part of the retina.

When there is a tear in the retina, or when there is traction causing separation of the retina from its underlying structures, liquid vitreous passes through the opening and into the subretinal space, inducing further exudation in the subretinal space. The retina can gradually separate and detach from the underlying retinal pigment epithelium. This deprives the outer retina of its normal supply of oxygen and nutrients from the choroid, and can result in damage to the retina.

Treatment of retinal detachment involves reestablishing the connection between the sensory retina and its underlying supporting tissue. If a detached retina is not timely repaired, the retinal pigment epithelium and glial cells can proliferate, forming fibrous bands under and in front of the retina which hold the retina in a fixed and detached position. In surgical repair of a detached retina, vitreous gel that fills the eye is removed, thereby permitting surgical access to the retinal tissue, and a tamponade agent is placed in the eye to apply force to the retina, thereby keeping retinal tissue in its desired location while the retina heals.

Tamponade agents commonly used in current medical practice include an expansive intraocular gas or silicone oil. Intraocular gas is the most commonly used form of retinal tamponade. When an intraocular gas is injected into the eye, it slowly expands to several times its initial volume. To keep the central portion of the retina attached, patients are required to be positioned face down for 2-6 weeks after surgery so that the gas bubble is directed upwards against the center of the retina. This requirement places a significant burden on patients. Another limitation of a gas tamponade is its inability to tamponade inferior pathology (retinal breaks/detachments in the bottom half of the eye) as the gas bubble rises in the eye. Currently there is no way to tamponade inferior retinal pathologies. Furthermore, use of gas in the eye prohibits patients from air travel or from receiving some inhalational anesthetic agents for up to 8 weeks. In addition, the gas causes a temporary but profound refractive shift (refractive index is <1.2, very much lower than that of the vitreous) which results in poor vision for up to 8 weeks until the gas bubble is absorbed.

The specific gravity of silicone oil is 0.97 g/cm3, which is slightly less than that of the normal eye fluid, making the oil slightly buoyant and resulting in a poor retinal tamponade effect. Retinal re-detachments are common when oil is in the eye due to the weak tamponade force that oil applies against the retina. In addition, the refractive index (>1.4) of the oil is in excess of that of the native vitreous, causing refractive error shifts of 5-10 diopters when the oil is in the eye. Furthermore, unlike gas, which essentially disappears on its own over several weeks, silicone oil removal requires a second surgery in the operating room for removal. In addition, silicone oil in many patients leads to keratopathy, glaucoma, and cataract formation.

Thus, both intraocular gas and oil have major limitations in both their function and in the burden they impose on the subject or patient. For intraocular gas, the limitations include: 1) face-down positioning of the subject or patient for several weeks after surgery; 2) poor effectiveness when the retinal pathology is in the bottom half of the retina; 3) poor post-operative vision; and 4) no travel by airplane for several months. For silicone oil, while it can be used when positioning is not possible or air travel is needed, it is nevertheless a poor tamponade agent and requires a second surgery for removal.

Many different tamponade agents have been investigated; however, they are often limited in there as a tamponade agent due to, for example, toxicity, emulsification, inadequate degradation rates, and/or being proinflammatory. The use of certain hydrogels has also been proposed in the past; however, those tested have run into various limitations, including lack of sufficient biocompatibility in the eye and the inability to inject the hydrogels through small needles so that the polymer does not shear or lose viscosity.

One significant limitation of certain hydrogels has been their strong promotion of an inflammatory response, including proliferation of fibrous membranes, recruitment of phagocytes that degrade the gel, and/or toxicity to the photoreceptors, as measured by decreased ERG amplitudes.

Additional limitations of certain hydrogel formulations include the tendency to shear and lose elasticity after injection through a small bore needle or to simply aggregate and/or loss of surface tension that permitted the gel to drift underneath retinal tears.

For some hydrogels, it has not demonstrated whether they could provided sufficient tamponade force, the implantation of the polymer was traumatic and took too long to swell into equilibrium, and/or sheer thinning occurred during injection due to a low degree of crosslinking.

Accordingly, the need exists for new methods for repairing retinal detachments, retinal tears, macular holes and related retinal disorders using new materials as a tamponade agent. A need exists for a retinal tamponade agent that would decrease patient morbidity (due to the need for repeat surgery when using silicone oil) and improve patient compliance and comfort (avoiding the face-down positioning when using intraocular gases). Such a tamponade agent would desirably apply an outward intraocular force in all directions, expanding in 360-degrees to remove the need for restrictive patient position, and be biodegradable and absorbable. The present invention addresses these needs and provides other advantages, including biocompatibility, desirable degradation rates, lack of emulsification, amenability to injection through small needles, sufficient surface tension, no impact (or minimal impact) on vision, no restrictions on subject position, and lack of toxicity.

### SUMMARY

Methods, polymer-containing formulations, and polymer compositions for treating retinal detachment and other ocular disorders, where the methods employ polymer compositions or polymer-containing formulations that can form a hydrogel in the eye of a subject are provided. Also provided are ocular formulations containing a polymer composition that can form a hydrogel in the eye of a subject. In certain embodiments, the hydrogel is formed by reaction of (a) a nucleo-functional polymer that is a biocompatible polymer containing (i) plurality of —OH groups, (ii) a plurality of thio-functional groups —R¹—SH wherein R¹ is an ester-containing linker, (iii) at least one polyethylene glycol group, and (iv) optionally one or more —OC(O)—(C¹-C⁶ alkyl) groups and (b) an electro-functional polymer that is a biocompatible polymer containing at least one thiol-reactive group, such as an alpha-beta unsaturated ester. In certain embodiments, the hydrogel is formed by reaction of (a) a nucleo-functional polymer is a biocompatible polyalkylene polymer substituted by (i) a plurality of —OH groups, (ii) a plurality of thio-functional groups —R¹—SH wherein R¹ is an ester-containing linker, and (iii) optionally one or more —OC(O)—(C₁-C₆ alkyl) groups, such as a thiolated poly(vinyl alcohol) polymer and (b) an electro-functional polymer that is a biocompatible polymer containing at least one thiol-reactive group, such as a poly(ethylene glycol) polymer containing alpha-beta unsaturated ester groups. Formulations are provided containing a nucleo-functional polymer, a poly(ethylene glycol) polymer, and an aqueous pharmaceutically acceptable carrier, for use in the therapeutic methods. In certain embodiments, the methods involve administering to the eye of the subject (a) a nucleo-functional polymer that is a biocompatible polymer containing (i) plurality of —OH groups, (ii) a plurality of thio-functional groups —R¹—SH wherein R¹ is an ester-containing linker, (iii) at least one polyethylene glycol group, and (iv) optionally one or more —OC(O)—(C¹-C⁶ alkyl) groups and (b) an electro-functional polymer that is a biocompatible polymer containing at least one thiol-reactive group, such as an alpha-beta unsaturated ester. The nucleo-functional polymer and electro-functional polymer are desirably low-viscosity materials that can be injected easily into the eye of a patient through a narrow-gauge needle, thereby permitting administration of the polymers through small surgical ports in the eye of the patient. This minimizes trauma to the patient's eye and is surgically feasible. The nucleo-functional polymer and electro-functional polymer begin to react spontaneously once mixed, where the vast majority of reaction between the nucleo-functional polymer and electro-functional polymer occurs while the polymers are in the patient's eye thereby forming a hydrogel in the eye of the patient that will apply pressure to and support retinal tissue in the eye of the patient.

In certain embodiments, the methods involve administering to the eye of the subject a biocompatible polymer and curing the biocompatible polymer to form a hydrogel in the vitreous cavity of the subject's eye. A biocompatible polymer may be exposed to a curing agent to facilitate curing of the biocompatible polymer to form the hydrogel. Depending on the identity of the biocompatible polymer, the curing agent may be heat, acid, an ion, a compound with one or more electrophilic groups, a compound with one or more nucleophilic groups, an enzyme, or other agent that facilitates formation of the hydrogel. In certain embodiments, the biocompatible functional polymer is a low-viscosity material that can be injected easily into the eye of a subject through a narrow-gauge needle, thereby permitting administration of the polymer through small surgical ports in the eye of the subject. This minimizes trauma to the subject's eye and is surgically feasible. Further features of the hydrogel may include: formation of the hydrogel uses materials that are non-toxic and no toxic by-products are formed by formation of the hydrogel, and the hydrogel undergoes biodegradation at a rate appropriate to support the retinal tissue over the timeframe necessary for healing of the retinal tissue. The appropriate biodegradation rate is advantageous because, for example, natural clearance of the hydrogel from the subject's eye at the appropriate time avoids having to perform a subsequent surgery to remove the hydrogel tamponade agent. Various aspects and embodiments of the invention are described in further detail below, along with further description of multiple advantages provided by the invention.

One exemplary advantage of certain methods and polymer compositions described herein is that no toxic initiator agent or ultra-violet light is required to facilitate reaction between the nucleo-functional polymer and electro-functional polymer. Additional exemplary advantages of methods and polymer compositions described herein is that reaction between the nucleo-functional polymer and electro-functional polymer does not generate byproducts or result in the formation of any medically significant heat. Thus, the methods and polymer compositions described herein are much safer than various polymer compositions described in literature previously. Still further exemplary advantages of the methods and polymer compositions described herein is that the polymers can be inserted through small surgical ports in the eye of the patient without causing any significant degradation of the polymer, and the resulting hydrogel formed by reaction of the polymers is non-toxic and undergoes biodegradation at a rate appropriate to support the retinal tissue over the timeframe necessary for healing of the retinal tissue. The appropriate biodegradation rate is advantageous because, for example, natural clearance of the hydrogel from the patient's eye at the appropriate time avoids having to perform a subsequent surgery to remove the hydrogel tamponade agent. Various aspects and embodiments of the invention are described in further detail below, along with further description of multiple advantages provided by the invention.

Accordingly, one aspect of the invention provides methods of contacting retinal tissue in the eye of a subject with a hydrogel. In certain embodiments, the method comprises (a) administering to the vitreous cavity of an eye of the subject an effective amount of (i) an electro-functional polymer and (ii) an ocular formulation comprising a nucleo-functional polymer, a poly(ethylene glycol) polymer, and an aqueous pharmaceutically acceptable carrier; and (b) allowing the nucleo-functional polymer and the electro-functional polymer to react to form a hydrogel in the vitreous cavity; wherein the nucleo-functional polymer is a biocompatible polyalkylene polymer substituted by (i) a plurality of —OH groups, (ii) a plurality of thio-functional groups —R¹—SH wherein R¹ is an ester-containing linker, and (iii) optionally one or more —OC(O)—(C₁-C₆ alkyl) groups; and wherein the electro-functional polymer is a biocompatible polymer containing at least one thiol-reactive group. In some embodiments, the method comprises (a) administering to the vitreous cavity of an eye of the subject an effective amount of a nucleo-functional polymer and an electro-functional polymer; and (b) allowing the nucleo-functional polymer and the electro-functional polymer to react to form a hydrogel in the vitreous cavity; wherein the nucleo-functional polymer is a biocompatible polyalkylene polymer substituted by (i) a plurality of —OH groups, (ii) a plurality of thio-functional groups —R¹—SH, (iii) at least one polyethylene glycolyl group, and (iv) optionally one or more —OC(O)—(C₁-C₆ alkyl) groups; R¹ is an ester-containing linker, and the electro-functional polymer is a biocompatible polymer containing at least one thiol-reactive group.

The nucleo-functional polymer and the electro-functional polymer may be administered together as a single composition to the vitreous cavity of the eye of the subject, or alternatively the nucleo-functional polymer and the electro-functional polymer may be administered separately to the vitreous cavity of the eye of the subject. The method may be further characterized according, for example, the identity of the nucleo-functional polymer, electro-functional polymer, and physical characteristics of the hydrogel formed therefrom, as described in the detailed description below. In certain embodiments, the method comprises (a) administering to the vitreous cavity of an eye of the subject an effective amount of a biocompatible polymer described herein, such as one of the thermosensitive polymers, nucleo-functional polymers, electro-functional polymers, pH-sensitive polymers, ion-sensitive polymers, photo-sensitive polymers, pressure-sensitive polymers, free-radical sensitive materials, or other materials described herein and (b) curing the biocompatible polymer to form a hydrogel in the vitreous cavity. The method may be further characterized according, for example, the identity of the biocompatible polymer, technique used to facilitate curing of the biocompatible polymer, and physical characteristics of the hydrogel formed therefrom, as described in the detailed description below. Exemplary subjects that may benefit from the method include, for example, subjects having a physical discontinuity in the retinal tissue, such as subjects having a tear in the retinal tissue, a break in the retinal tissue, or a hole in the retinal tissue. In certain embodiments, the subject has undergone surgery for a macular hole or has undergone a vitrectomy for vitreomacular traction. In certain other embodiments, the subject has undergone surgery to repair a serous retinal detachment, to repair a tractional retinal detachment, or to remove at least a portion of an epiretinal membrane.

Another aspect of the invention provides a method of supporting retinal tissue in the eye of a subject, the method comprising: (a) administering to the vitreous cavity of an eye of the subject an effective amount of (i) an electro-functional polymer and (ii) an ocular formulation comprising a nucleo-functional polymer, a poly(ethylene glycol) polymer, and an aqueous pharmaceutically acceptable carrier; and (b) allowing the nucleo-functional polymer and the electro-functional polymer to react to form a hydrogel in the vitreous cavity; wherein the nucleo-functional polymer is a biocompatible polyalkylene polymer substituted by (i) a plurality of —OH groups, (ii) a plurality of thio-functional groups —R¹—SH wherein R¹ is an ester-containing linker, and (iii) optionally one or more —OC(O)—(C₁-C₆ alkyl) groups; and wherein the electro-functional polymer is a biocompatible polymer containing at least one thiol-reactive group. In certain embodiments, the invention provides a method of supporting retinal tissue in the eye of a subject, the method comprising: (a) administering to the vitreous cavity of an eye of the subject an effective amount of a nucleo-functional polymer and an electro-functional polymer; and (b) allowing the nucleo-functional polymer and the electro-functional polymer to react to form a hydrogel in the vitreous cavity; wherein the nucleo-functional polymer is a biocompatible polyalkylene polymer substituted by (i) a plurality of —OH groups, (ii) a plurality of thio-functional groups —R¹—SH, (iii) at least one polyethylene glycolyl group, and (iv) optionally one or more —OC(O)—(C₁-C₆ alkyl) groups; R₁ is an ester-containing linker, and the electro-functional polymer is a biocompatible polymer containing at least one thiol-reactive group. The nucleo-functional polymer and the electro-functional polymer may be administered together as a single composition to the vitreous cavity of the eye of the subject, or alternatively the nucleo-functional polymer and the electro-functional polymer may be administered separately to the vitreous cavity of the eye of the subject. The method may be further characterized according, for example, the identity of the nucleo-functional polymer, electro-functional polymer, and physical characteristics of the hydrogel formed therefrom, as described in the detailed description below. Exemplary subjects that may benefit from the method include, for example, subjects having a physical discontinuity in the retinal tissue, such as subjects having a tear in the retinal tissue, a break in the retinal tissue, or a hole in the retinal tissue. In certain embodiments, the subject has undergone surgery for a macular hole or has undergone a vitrectomy for vitreomacular traction. In certain other embodiments, the subject has undergone surgery to repair a serous retinal detachment, to repair a tractional retinal detachment, or to remove at least a portion of an epiretinal membrane.

Another aspect of the invention provides a method of supporting retinal tissue in the eye of a subject, the method comprising: (a) administering to the vitreous cavity of an eye of the subject an effective amount of a biocompatible polymer described herein, such as one of the thermosensitive polymers, nucleo-functional polymers, electro-functional polymers, pH-sensitive polymers, ion-sensitive polymers, photo-sensitive polymers, pressure-sensitive polymers, free-radical sensitive materials, or other materials described herein and (b) curing the biocompatible polymer to form a hydrogel in the vitreous cavity. The method may be further characterized according, for example, the identity of the biocompatible polymer, technique used to facilitate curing of the biocompatible polymer, and physical characteristics of the hydrogel formed therefrom, as described in the detailed description below. Exemplary subjects that may benefit from the method include, for example, subjects having a physical discontinuity in the retinal tissue, such as subjects having a tear in the retinal tissue, a break in the retinal tissue, or a hole in the retinal tissue. In certain embodiments, the subject has undergone surgery for a macular hole or has undergone a vitrectomy for vitreomacular traction. In certain other embodiments, the subject has undergone surgery to repair a serous retinal detachment, to repair a tractional retinal detachment, or to remove at least a portion of an epiretinal membrane.

Another aspect of the invention provides a method of treating a subject with a retinal detachment, the method comprising: (a) administering to the vitreous cavity of an eye of the subject with a detachment of at least a portion of retinal tissue an effective amount of (i) an electro-functional polymer and (ii) an ocular formulation comprising a nucleo-functional polymer, a poly(ethylene glycol) polymer, and an aqueous pharmaceutically acceptable carrier; and (b) allowing the nucleo-functional polymer and the electro-functional polymer to react to form a hydrogel in the vitreous cavity; wherein the hydrogel supports the retinal tissue during reattachment of the portion of the retinal tissue; the nucleo-functional polymer is a biocompatible polyalkylene polymer substituted by (i) a plurality of —OH groups, (ii) a plurality of thio-functional groups —R¹—SH wherein R¹ is an ester-containing linker, and (iii) optionally one or more —OC(O)—(C₁-C₆ alkyl) groups; and the electro-functional polymer is a biocompatible polymer containing at least one thiol-reactive group. In certain embodiments, the invention provides a method of treating a subject with a retinal detachment, the method comprising: (a) administering an effective amount of a nucleo-functional polymer and an electro-functional polymer to the vitreous cavity of an eye of the subject with a detachment of at least a portion of retinal tissue; and (b) allowing the nucleo-functional polymer and the electro-functional polymer to react to form a hydrogel in the vitreous cavity; wherein the nucleo-functional polymer is a biocompatible polyalkylene polymer substituted by (i) a plurality of —OH groups, (ii) a plurality of thio-functional groups —R¹—SH, (iii) at least one polyethylene glycolyl group, and (iv) optionally one or more —OC(O)—(C₁-C₆ alkyl) groups; R¹ is an ester-containing linker, and the electro-functional polymer is a biocompatible polymer containing at least one thiol-reactive group. The nucleo-functional polymer and the electro-functional polymer may be administered together as a single composition to the vitreous cavity of the eye of the subject, or alternatively the nucleo-functional polymer and the electro-functional polymer may be administered separately to the vitreous cavity of the eye of the subject. The method may be further characterized according, for example, the identity of the nucleo-functional polymer, electro-functional polymer, and physical characteristics of the hydrogel formed therefrom, as described in the detailed description below. The retinal detachment may be, for example, a rhegmatogenous retinal detachment, a tractional retinal detachment, or a serous retinal detachment.

Another aspect of the invention provides a method of treating a subject with a retinal detachment, the method comprising: (a) administering to the vitreous cavity of an eye of the subject an effective amount of a biocompatible polymer described herein, such as one of the thermosensitive polymers, nucleo-functional polymers, electro-functional polymers, pH-sensitive polymers, ion-sensitive polymers, photo-sensitive polymers, pressure-sensitive polymers, free-radical sensitive materials, or other materials described herein and (b) curing the biocompatible polymer to form a hydrogel in the vitreous cavity. The method may be further characterized according, for example, the identity of the biocompatible polymer, technique used to facilitate curing of the biocompatible polymer, and physical characteristics of the hydrogel formed therefrom, as described in the detailed description below. Exemplary subjects that may benefit from the method include, for example, subjects having a physical discontinuity in the retinal tissue, such as subjects having a tear in the retinal tissue, a break in the retinal tissue, or a hole in the retinal tissue. In certain embodiments, the subject has undergone surgery for a macular hole or has undergone a vitrectomy for vitreomacular traction. In certain other embodiments, the subject has undergone surgery to repair a serous retinal detachment, to repair a tractional retinal detachment, or to remove at least a portion of an epiretinal membrane.

Another aspect of the invention provides an injectable, ocular formulation for forming a hydrogel in the eye of a subject, the formulation comprising: (a) a nucleo-functional polymer that is a biocompatible polyalkylene polymer substituted by (i) a plurality of —OH groups, (ii) a plurality of thio-functional groups —R¹—SH wherein R¹ is an ester-containing linker, and (iii) optionally one or more —OC(O)—(C₁-C₆ alkyl) groups; (b) a poly(ethylene glycol) polymer; and (c) aqueous pharmaceutically acceptable carrier for administration to the eye of a subject. In certain embodiments, the invention provides an injectable, ocular formulation for forming a hydrogel in the eye of a subject, the formulation comprising: (a) a nucleo-functional polymer that is a biocompatible polyalkylene polymer substituted by (i) a plurality of —OH groups, (ii) a plurality of thio-functional groups —R¹—SH, (iii) at least one polyethylene glycolyl group, and (iv) optionally one or more —OC(O)—(C₁-C₆ alkyl) groups; R¹ is an ester-containing linker; (b) an electro-functional polymer that is a biocompatible polymer containing at least one thiol-reactive group; and (c) a liquid pharmaceutically acceptable carrier for administration to the eye of a subject. In some embodiments, the invention provides an injectable, ocular formulation for forming a hydrogel in the eye of a subject, the formulation comprising: (a) a biocompatible polymer described herein, such as one of the thermosensitive polymers, nucleo-functional polymers, electro-functional polymers, pH-sensitive polymers, ion-sensitive polymers, photo-sensitive polymers, pressure-sensitive polymers, free-radical sensitive materials, or other materials described herein and (b) a liquid pharmaceutically acceptable carrier for administration to the eye of a subject. Such injectable, ocular formulation for forming a hydrogel may be used in the methods described herein.

In certain embodiments, the nucleo-functional polymer may be, for example, a biocompatible poly(vinyl alcohol) polymer substituted by a plurality of thio-functional groups —R¹—SH. In certain embodiments, the nucleo-functional polymer is a biocompatible poly(vinyl alcohol) polymer comprising:

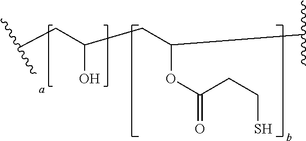

wherein a is an integer from 1-10 and b is an integer from 1-10.

The electro-functional polymer may be, for example, a biocompatible polymer selected from a polyalkylene and polyheteroalkylene polymer each being substituted by at least one thiol-reactive group. In certain embodiments, the thiol-reactive group is —OC(O)CH═CH₂. In yet other embodiments, the electro-functional polymer has the formula:

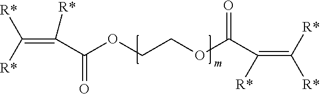

wherein R* is independently for each occurrence hydrogen, alkyl, aryl, or aralkyl; and m is an integer in the range of 5 to 15,000.

Another aspect of the invention provides an polyalkylene polymer substituted by (i) a plurality of —OH groups, (ii) a plurality of thio-functional groups —R¹—SH, (iii) at least one polyethylene glycolyl group, and (iv) optionally one or more —OC(O)—(C₁-C₆ alkyl) groups; R¹ is an ester-containing linker. In certain embodiments, the polymer is a poly(vinyl alcohol) polymer substituted by (i) a plurality of thio-functional groups —R¹—SH and (ii) at least one polyethylene glycolyl group.

In certain embodiments, the hydrogels described herein include one or more of the following properties: 1) provides a tamponade force in 360-degrees (a comprehensive agent for all retinal pathologies) by providing increased pressure inside the eye to force the retina out against the sclera; 2) has a high surface tension for preventing the agent from getting under the breaks in the retina or breaking up into smaller pieces; 3) has a relatively low viscosity such that the substance could be injected over several minutes through a small bore needle (e.g., 25 gauge needle) and/or be cross-linked inside the eye; 4) is degradable and provides a continuous tamponade force for a desirable amount of time (e.g., less than about 30 days) and/or may be susceptible to induced degradation, such as an injection of an agent into the eye that induces degradation, to an absorbable byproduct; 5) is biologically inert; and 6) has an index of refraction similar to water (e.g., 1.3) that would allow the subject to see clearly while the substance is in place.

### DETAILED DESCRIPTION OF THE INVENTION

Methods, polymer-containing formulations, and polymer compositions for treating retinal detachment and other ocular disorders, where the methods employ polymer compositions that can form a hydrogel in the eye of a subject, are provided. Achieving a suitable tamponade agent is difficult, in part because the material needs to meet multiple criteria, which include that it be easily administered to the eye, that once in eye the material provides sufficient support/pressure on the entire retina, the material is not toxic to the subject, the material is desirably optically clear, and the material undergoes biodegradation at an appropriate rate so that the retinal tissue is supported for an appropriate amount of time to facilitate healing of retinal tissue following a vitrectomy without having to perform a second surgery to remove the tamponade agent.

In certain embodiments, the hydrogel is formed by reaction of (a) a nucleo-functional polymer that is a biocompatible polyalkylene polymer substituted by (i) a plurality of —OH groups, (ii) a plurality of thio-functional groups —R¹—SH wherein R¹ is an ester-containing linker, and (iii) optionally one or more —OC(O)—(C₁-C₆ alkyl) groups, such as a thiolated poly(vinyl alcohol) polymer and (b) an electro-functional polymer that is a biocompatible polymer containing at least one thiol-reactive group, such as a poly(ethylene glycol) polymer containing alpha-beta unsaturated ester groups. Formulations are provided containing a nucleo-functional polymer, a poly(ethylene glycol) polymer, and an aqueous pharmaceutically acceptable carrier, for use in the therapeutic methods. In some embodiments, the methods involve administering to the eye of the subject (a) a nucleo-functional polymer that is a biocompatible polymer containing (i) plurality of —OH groups, (ii) a plurality of thio-functional groups —R¹—SH wherein R¹ is an ester-containing linker, (iii) at least one polyethylene glycolyl group, and (iv) optionally one or more —OC(O)—(C¹-C⁶ alkyl) groups and (b) an electro-functional polymer that is a biocompatible polymer containing at least one thiol-reactive group, such as an alpha-beta unsaturated ester. The nucleo-functional polymer and electro-functional polymer are desirably low-viscosity materials that can be injected easily into the eye of a patient through a narrow-gauge needle, thereby permitting administration of the polymers through small surgical ports in the eye of the patient. This minimizes trauma to the patient's eye. The nucleo-functional polymer and electro-functional polymer begin to react spontaneously once mixed, where the vast majority of reaction between the nucleo-functional polymer and electro-functional polymer occurs while the polymers are in the patient's eye thereby forming a hydrogel in the eye of the patient that will apply pressure to and support retinal tissue in the eye of the patient. In certain embodiments, the hydrogel describe herein is a crosslinked hydrogel formed in situ to create a temporary synthetic vitreous for retinal tamponade in vitreoretinal surgery. In some embodiments, crosslinking may be achieved by mixing two solutions just prior to injection into the eye. The mixed solution is then injected into the eye by the surgeon after fluid-air exchange. In certain embodiments, the hydrogel forms in the eye within several minutes of mixing and prevents fluid leakage behind the retina following repair. In some embodiments, the hydrogel then degrades into components that can be safely eliminated from the eye.

In certain embodiments of the methods and polymer compositions described herein, no toxic initiator agent or ultra-violet light is required to facilitate reaction between the nucleo-functional polymer and electro-functional polymer. In some embodiments, exemplary advantages of methods and polymer compositions described herein is that reaction between the nucleo-functional polymer and electro-functional polymer does not generate byproducts or result in the formation of any medically significant heat. Thus, in certain embodiments the methods and polymer compositions described herein are much safer than various polymer compositions described in literature previously. Still further exemplary advantages of the methods and polymer compositions described herein is that the polymers can be inserted through small surgical ports in the eye of the patient without causing any significant degradation of the polymer, and the resulting hydrogel formed by reaction of the polymers is non-toxic and undergoes biodegradation at a rate appropriate to support the retinal tissue over the timeframe necessary for healing of the retinal tissue. The appropriate biodegradation rate is advantageous because, for example, natural clearance of the hydrogel from the patient's eye at the appropriate time avoids having to perform a subsequent surgery to remove the hydrogel tamponade agent.

The invention also provides methods comprising administering to the eye of the subject a biocompatible polymer and curing the biocompatible polymer to form a hydrogel in the vitreous cavity of the subject's eye. A biocompatible polymer is may be exposed to a curing agent to facilitate curing of the biocompatible polymer to form the hydrogel. Depending on the identity of the biocompatible polymer, the curing agent may be heat, acid, an ion, a compound with one or more electrophilic groups, a compound with one or more nucleophilic groups, an enzyme, or other agent that facilitates formation of the hydrogel. For example, the biocompatible functional polymer is a low-viscosity material that can be injected easily into the eye of a subject through a narrow-gauge needle, thereby permitting administration of the polymer through small surgical ports in the eye of the subject. This minimizes trauma to the subject's eye and is surgically feasible. Further features of the hydrogel include: formation of the hydrogel uses materials that are non-toxic and no toxic by-products are formed by formation of the hydrogel, and the hydrogel undergoes biodegradation at a rate appropriate to support the retinal tissue over the timeframe necessary for healing of the retinal tissue. The appropriate biodegradation rate is advantageous because, for example, natural clearance of the hydrogel from the subject's eye at the appropriate time avoids having to perform a subsequent surgery to remove the hydrogel tamponade agent.

Various aspects of the invention are set forth below in sections; however, aspects of the invention described in one particular section are not to be limited to any particular section.

### I. Definitions

To facilitate an understanding of the present invention, a number of terms and phrases are defined below.

The terms “a” and “an” as used herein mean “one or more” and include the plural unless the context is inappropriate.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, and C₁-C₆alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈cycloalkyl,” derived from a cycloalkane. Exemplary cycloalkyl groups include, but are not limited to, cyclohexanes, cyclopentanes, cyclobutanes and cyclopropanes.

The term “aryl” is art-recognized and refers to a carbocyclic aromatic group. Representative aryl groups include phenyl, naphthyl, anthracenyl, and the like. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “aryl” also includes polycyclic ring systems having two or more carbocyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, and/or aryls. In certain embodiments, the aromatic ring is substituted at one or more ring positions with halogen, alkyl, hydroxyl, or alkoxyl. In certain other embodiments, the aromatic ring is not substituted, i.e., it is unsubstituted.

The term “aralkyl” refers to an alkyl group substituted with an aryl group.

The term “heteroaryl” is art-recognized and refers to aromatic groups that include at least one ring heteroatom. In certain instances, a heteroaryl group contains 1, 2, 3, or 4 ring heteroatoms. Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic ring systems having two or more rings in which two or more carbons are common to two adjoining rings (the rings are “fused rings”) wherein at least one of the rings is heteroaromatic, e.g., the other cyclic rings may be cycloalkyls, cycloalkenyls, cycloalkynyls, and/or aryls. In certain embodiments, the heteroaryl ring is substituted at one or more ring positions with halogen, alkyl, hydroxyl, or alkoxyl. In certain other embodiments, the heteroaryl ring is not substituted, i.e., it is unsubstituted.

The term “heteroaralkyl” refers to an alkyl group substituted with a heteroaryl group.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl” and “heterocyclic group” are art-recognized and refer to saturated or partially unsaturated 3- to 10-membered ring structures, alternatively 3- to 7-membered rings, whose ring structures include one to four heteroatoms, such as nitrogen, oxygen, and sulfur. The number of ring atoms in the heterocyclyl group can be specified using Cₓ—Cₓ nomenclature where x is an integer specifying the number of ring atoms. For example, a C₃-C₇heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position. One example of a C₃heterocyclyl is aziridinyl. Heterocycles may also be mono-, bi-, or other multi-cyclic ring systems. A heterocycle may be fused to one or more aryl, partially unsaturated, or saturated rings. Heterocyclyl groups include, for example, biotinyl, chromenyl, dihydrofuryl, dihydroindolyl, dihydropyranyl, dihydrothienyl, dithiazolyl, homopiperidinyl, imidazolidinyl, isoquinolyl, isothiazolidinyl, isoxazolidinyl, morpholinyl, oxolanyl, oxazolidinyl, phenoxanthenyl, piperazinyl, piperidinyl, pyranyl, pyrazolidinyl, pyrazolinyl, pyridyl, pyrimidinyl, pyrrolidinyl, pyrrolidin-2-onyl, pyrrolinyl, tetrahydrofuryl, tetrahydroisoquinolyl, tetrahydropyranyl, tetrahydroquinolyl, thiazolidinyl, thiolanyl, thiomorpholinyl, thiopyranyl, xanthenyl, lactones, lactams such as azetidinones and pyrrolidinones, sultams, sultones, and the like. Unless specified otherwise, the heterocyclic ring is optionally substituted at one or more positions with substituents such as alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl and thiocarbonyl. In certain embodiments, the heterocyclcyl group is not substituted, i.e., it is unsubstituted.

The terms “amine” and “amino” are art-recognized and refer to both unsubstituted and substituted amines, e.g., a moiety represented by the general formula —N(R⁵⁰)(R⁵¹), wherein R⁵⁰ and R⁵¹ each independently represent hydrogen, alkyl, cycloalkyl, heterocyclyl, alkenyl, aryl, aralkyl, or —(CH₂)ₘ—R⁶¹; or R⁵⁰ and R⁵¹, taken together with the N atom to which they are attached complete a heterocycle having from 4 to 8 atoms in the ring structure; R⁶¹ represents an aryl, a cycloalkyl, a cycloalkenyl, a heterocycle or a polycycle; and m is zero or an integer in the range of 1 to 8. In certain embodiments, R⁵⁰ and R⁵¹ each independently represent hydrogen, alkyl, alkenyl, or —(CH₂)ₘ—R⁶¹.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R₆₁, where m and R₆₁ are described above.

The term “amide” or “amido” as used herein refers to a radical of the form —RₐC(O)N(R_{b})—, —RₐC(O)N(R_{b})R_{c}—, —C(O)NR_{b}R_{c}, or —C(O)NH₂, wherein Rₐ, R_{b} and R_{c} are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro. The amide can be attached to another group through the carbon, the nitrogen, R_{b}, R_{c}, or Rₐ. The amide also may be cyclic, for example R_{b} and R_{c}, Rₐ and R_{b}, or Rₐ and Rₑ may be joined to form a 3- to 12-membered ring, such as a 3- to 10-membered ring or a 5- to 6-membered ring.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

As used herein, the terms “subject” and “patient” refer to organisms to be treated by the methods of the present invention. Such organisms are preferably mammals (e.g., murines, simians, equines, bovines, porcines, canines, felines, and the like), and more preferably humans.

As used herein, the term “effective amount” refers to the amount of a compound (e.g., a compound of the present invention) sufficient to effect beneficial or desired results. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. In certain embodiments, the pharmaceutically acceptable carrier is, or comprises, balanced salt solution. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. [1975]. The compositions may optionally contain a dye. Accordingly, in certain embodiments, the composition further comprises a dye.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### II. Therapeutic Methods and Injectable, Ocular Formulations for Forming a Hydrogel

Methods, polymer-containing formulations, and polymer compositions for treating retinal detachment and other ocular disorders, where the methods employ polymer formulations or compositions that can form a hydrogel in the eye of a subject, are provided. Also provided are ocular formulations containing a polymer composition that can form a hydrogel in the eye of a subject. The methods include, for example, methods for contacting retinal tissue in the eye of a subject with a hydrogel, methods for supporting retinal tissue, methods for treating a subject with a retinal detachment, and methods for treating hypotony, methods for treating a choroidal effusion, methods for supporting tissue in or adjacent to the anterior chamber of the eye, and methods of maintaining or expanding a nasolacrimal duct, and injectable, ocular formulations for forming a hydrogel.

In certain embodiments, the polymer compositions include polyalkylene polymers substituted by (i) a plurality of —OH groups, (ii) a plurality of thio-functional groups —R¹—SH, (iii) at least one polyethylene glycolyl group, and (iv) optionally one or more —OC(O)—(C₁-C₆ alkyl) groups; R¹ is an ester-containing linker. Multiple features and embodiments of the polyalkylene polymers are described herein below, which include embodiments where, for example, the polymer is a poly(vinyl alcohol) polymer substituted by (i) a plurality of thio-functional groups —R¹—SH and (ii) at least one polyethylene glycolyl group. In certain embodiments, the polymer is a partially hydrolyzed poly(vinyl alcohol) polymer substituted by (i) a plurality of thio-functional groups —R¹—SH and (ii) at least one polyethylene glycolyl group. Such partially hydrolyzed polymer can be characterized by the degree of hydrolysis, such as where the degree of hydrolysis of the partially hydrolyzed poly(vinyl alcohol) polymer is at least 85%, or where the degree of hydrolysis of the partially hydrolyzed poly(vinyl alcohol) polymer is at least 95%. In certain embodiments, —R¹—SH is —OC(O)—(C₁-C⁶ alkylene)-SH. In certain other embodiments, —R¹—SH is —OC(O)—(CH₂CH₂)—SH.

The methods, formulations, and compositions are described in more detail below.

### First Embodiment—Contacting Retinal Tissue in the Eye of a Subject with a Hydrogel

One aspect of the invention provides a method of contacting retinal tissue in the eye of a subject with a hydrogel. In certain embodiments, the method comprises (a) administering to the vitreous cavity of an eye of the subject an effective amount of (i) an electro-functional polymer and (ii) an ocular formulation comprising a nucleo-functional polymer, a poly(ethylene glycol) polymer, and an aqueous pharmaceutically acceptable carrier; and (b) allowing the nucleo-functional polymer and the electro-functional polymer to react to form a hydrogel in the vitreous cavity; wherein the nucleo-functional polymer is a biocompatible polyalkylene polymer substituted by (i) a plurality of —OH groups, (ii) a plurality of thio-functional groups —R¹—SH wherein R¹ is an ester-containing linker, and (iii) optionally one or more —OC(O)—(C₁-C₆ alkyl) groups; and wherein the electro-functional polymer is a biocompatible polymer containing at least one thiol-reactive group. In some embodiments, the method comprises (a) administering to the vitreous cavity of an eye of the subject an effective amount of a nucleo-functional polymer and an electro-functional polymer; and (b) allowing the nucleo-functional polymer and the electro-functional polymer to react to form a hydrogel in the vitreous cavity; wherein the nucleo-functional polymer is a biocompatible polyalkylene polymer substituted by (i) a plurality of —OH groups, (ii) a plurality of thio-functional groups —R¹—SH, (iii) at least one polyethylene glycolyl group, and (iv) optionally one or more —OC(O)—(C₁-C₆ alkyl) groups; R¹ is an ester-containing linker, and the electro-functional polymer is a biocompatible polymer containing at least one thiol-reactive group.

The nucleo-functional polymer and an electro-functional polymer are administered to the eye of the subject in an amount effective to produce a hydrogel that contacts retinal tissue. This effective amount may vary depending on the volume of the eye cavity to be filled, such that a large eye cavity will require more nucleo-functional polymer and an electro-functional polymer to produce a hydrogel occupying more volume, as can be readily determined by those of skill in the art based on the teachings provided herein. In certain embodiments, the volume of the hydrogel solution (e.g., the amount of the nucleo-functional polymer and electro-functional polymer administered separately or together) administered to the eye is sufficient to fill the cavity one eye. In some embodiments, the amount volume of hydrogel solution administered to the cavity of the eye is about 1 mL, 2 mL, 3 mL, 4 mL, 5 mL, 6 mL, or 7 mL. In certain embodiments, the amount of hydrogel solution administered to the cavity of the eye is at least 6 mL.

In certain embodiments, the nucleo-functional polymer and the electro-functional polymer are administered separately to the vitreous cavity of the eye of the subject. In certain embodiments, the electro-functional polymer is administered as a liquid pharmaceutical formulation containing an aqueous pharmaceutically acceptable carrier to the vitreous cavity of the eye of the subject.

The method can also be further characterized by, for example, the identity of the nucleo-functional polymer, the identity of the electro-functional polymer, the identity of the poly(ethylene glycol) polymer, physical characteristics of the hydrogel formed, and/or other features described herein below.

In certain embodiments, the method comprises:

(a) administering to the vitreous cavity of an eye of the subject an effective amount of a biocompatible polymer selected from the group consisting of:
i. a thermosensitive polymer selected from a hydroxybutyl chitosan, carboxymethyl chitosan, chitosan-(D)-glucose phosphate, (chitosan)-(hydroxypropylmethyl cellulose)-(glycerin) polymer, chitosan-(beta-gly cerophosphate)-hydroxyethyl cellulose polymer, (hyaluronic acid)-(hyperbranched polyethylene glycol) copolymer, poloxamer, (poloxamer)-(chondroitan sulfate)-(polyethylene glycol) polymer, (poly(lactic acid))-(poloxamer)-(poly(lactic acid) polymer, (polyethylene glycol)-polyalanine copolymer, (polyethylene glycol)-(poly caprolactone)-(polyethylene glycol) polymer, (polyethylene glycol)-(polyester urethane) copolymer, [poly(beta-benzyl L-aspartate)]-(polyethylene glycol)-[poly(beta-benzyl L-aspartate)], polycaprolactone-(polyethylene glycol)-polycaprolactone polymer, poly(lactic-co-glycolic acid)-(polyethylene glycol)-(poly(lactic-co-glycolic acid)), polymethacrylamide-polmethacrylate copolymer, poly(methacrylamide-co-methacrylate)-gellan gum copolymer, thiolated gellan, acrylated poloxamine, poly(N-isopropylacrylamide), poly(phosphazene), collagen-(poly(glycolic acid)) copolymer, (glycosaminoglycan)-(polypeptide) polymer, (ulvan)-(polyisopropylacrylamide) copolymer, a mixture of poloxamers, a mixture of hyaluronic acid and (polycaprolactone-(polyethylene glycol)-polycaprolactone), and mixtures thereof;
ii. a nucleo-functional polymer selected from a N—O carboxymethyl chitosan, (poloxamer)-(chondroitan sulfate)-(polyethylene glycol) polymer, polyethylene glycol, (hyaluronic acid)-(polygalacturonic acid) copolymer, (hyaluronic acid)-(gelatin)-(polyethylene glycol) polymer, (hyaluronic acid)-(collagen)-(sericin) polymer, (hyaluronic acid)-dextran copolymer, star polyethylene glycol, (star polyethylene glycol)-dextran copolymer, lysine-functionalized polyethylene glycol, (polyethylene glycol)-(dendritic lysine) polymer, polyethylene glycol-polylysine copolymer, thioloated gellan, acylated-sulfobetaine-starch, acrylated poloxamine, polyamidoamine dendrimer, (polyamidoamine dendrimer)-dextran copolymer, chitosan-dextran copolymer, chitosan-alginate copolymer, (carboxymethyl chitosan)-(carboxymethyl cellulose) copolymer, hyaluronic acid, tetra-succinimidyl substituted polyethylene glycol, tetra-thiol-substituted polyethylene glycol, and mixtures thereof;
iii. an electro-functional polymer selected from a (polyethylene glycol)-(dendritic thioester) polymer, acrylated four-arm polymer containing (poly(p-phenylene oxide))-(polyethylene glycol)-(poly(p-phenylene oxide)), poly(methacrylamide-co-methacrylate)-gellan gum copolymer, chitosan-polylysine copolymer, hyaluronic acid, and mixtures thereof;
iv. a pH-sensitive polymer selected from (polyethylene glycol)-polyaspartylhydrazide copolymer, chitosan-alginate copolymer, chitosan-(gellan gum) copolymer, and mixtures thereof;
v. an ion-sensitive polymer selected from an alginate-chitosan-genipin polymer, chitosan-alginate copolymer, chitosan-(gellan gum) copolymer, gellan gum-kappa carrageenan copolymer, and mixtures thereof,
vi. a photo-sensitive polymer selected from a (polyethylene glycol)-lactide, (polyethylene glycol)-fibrinogen polymer, acrylate-(polyethylene glycolyl)-acrylate, alginate, gelatin, pHEMA-co-APMA-polyamidoamine, poly(6-aminohexyl propylene phosphate), carboxymethyl chitan, hyaluronic acid, and mixtures thereof,
vii. an enzyme-reactive polymer selected from a (polylysine)-(polyethylene glycol)-tyramine polymer, gelatin, pullulan, poly(phenylene oxide)-polyethylene glycol copolymer, gelatin-chitosan copolymer, and mixtures thereof;
viii. a pressure-sensitive polymer selected from (polyethylene glycol)-dihydroxyacetone;
ix. free-radical sensitive polymer selected from a betaine-containing polymer;
x. a polymer selected from a (carboxymethylchitosan)-(oxidized alginate) copolymer, hyaluronic acid, (hyaluronic acid)-(crosslinked alginate) copolymer, (vinyl phosphonic acid)-acrylamide polymer, (poly(vinyl alcohol))-(carboxymethyl cellulose) copolymer, and mixtures thereof, and
xi. mixtures thereof, and
   (b) curing the biocompatible polymer to form a hydrogel in the vitreous cavity.

In certain embodiments, the curing comprises administering a curing agent to the vitreous cavity of an eye of the subject to facilitate curing of the biocompatible polymer. In certain embodiments, the biocompatible polymer is exposed to a curing agent prior to administering the biocompatible polymer to the vitreous cavity of the eye of the subject. In certain embodiments, the biocompatible polymer and a curing agent are administered concurrently to the vitreous cavity of the eye of the subject.

The biocompatible polymer is administered to the eye of the subject in an amount effective to produce a hydrogel that contacts retinal tissue. This effective amount may vary depending on the volume of the eye cavity to be filled, such that a large eye cavity will require more biocompatible polymer to produce a hydrogel occupying more volume, as can be readily determined by those of skill in the art based on the teachings provided herein.

The method can also be further characterized by, for example, the identity of the biocompatible polymer, presence and identity of a curing agent, physical characteristics of the hydrogel formed, and/or other features described herein below.

The method can be further characterized by, for example, the identity of the subject. In certain embodiments, subject has a physical discontinuity in the retinal tissue. In certain embodiments, the physical discontinuity is a tear in the retinal tissue, a break in the retinal tissue, or a hole in the retinal tissue. In other embodiments, the subject has undergone surgery for a macular hole, has undergone surgery to remove at least a portion of a epiretinal membrane, or has undergone a vitrectomy for vitreomacular traction. In other embodiments, the subject has a detachment of at least a portion of the retinal tissue. The retinal detachment may be, for example, a rhegmatogenous retinal detachment. Alternatively, the retinal detachment may be tractional retinal detachment or serous retinal detachment.

### Second Embodiment—Supporting Retinal Tissue

Another aspect of the invention provides a method of supporting retinal tissue in the eye of a subject, the method comprising: (a) administering to the vitreous cavity of an eye of the subject an effective amount of (i) an electro-functional polymer and (ii) an ocular formulation comprising a nucleo-functional polymer, a poly(ethylene glycol) polymer, and an aqueous pharmaceutically acceptable carrier; and (b) allowing the nucleo-functional polymer and the electro-functional polymer to react to form a hydrogel in the vitreous cavity; wherein the nucleo-functional polymer is a biocompatible polyalkylene polymer substituted by (i) a plurality of —OH groups, (ii) a plurality of thio-functional groups —R¹—SH wherein R¹ is an ester-containing linker, and (iii) optionally one or more —OC(O)—(C₁-C₆ alkyl) groups; and wherein the electro-functional polymer is a biocompatible polymer containing at least one thiol-reactive group. In some embodiments, the invention provides a method of supporting retinal tissue in the eye of a subject, the method comprising: (a) administering to the vitreous cavity of an eye of the subject an effective amount of nucleo-functional polymer and an electro-functional polymer; and (b) allowing the nucleo-functional polymer and the electro-functional polymer to react to form a hydrogel in the vitreous cavity; wherein the nucleo-functional polymer is a biocompatible polyalkylene polymer substituted by (i) a plurality of —OH groups, (ii) a plurality of thio-functional groups —R¹—SH, (iii) at least one polyethylene glycolyl group, and (iv) optionally one or more —OC(O)—(C₁-C₆ alkyl) groups; R¹ is an ester-containing linker, and the electro-functional polymer is a biocompatible polymer containing at least one thiol-reactive group.

In certain embodiments, the method comprises:

(a) administering to the vitreous cavity of an eye of the subject an effective amount of a biocompatible polymer selected from the group consisting of:
i. a thermosensitive polymer selected from a hydroxybutyl chitosan, carboxymethyl chitosan, chitosan-(D)-glucose phosphate, (chitosan)-(hydroxypropylmethyl cellulose)-(glycerin) polymer, chitosan-(beta-gly cerophosphate)-hydroxyethyl cellulose polymer, (hyaluronic acid)-(hyperbranched polyethylene glycol) copolymer, poloxamer, (poloxamer)-(chondroitan sulfate)-(polyethylene glycol) polymer, (poly(lactic acid))-(poloxamer)-(poly(lactic acid) polymer, (polyethylene glycol)-polyalanine copolymer, (polyethylene glycol)-(poly caprolactone)-(polyethylene glycol) polymer, (polyethylene glycol)-(polyester urethane) copolymer, [poly(beta-benzyl L-aspartate)]-(polyethylene glycol)-[poly(beta-benzyl L-aspartate)], polycaprolactone-(polyethylene glycol)-polycaprolactone polymer, poly(lactic-co-glycolic acid)-(polyethylene glycol)-(poly(lactic-co-glycolic acid)), polymethacrylamide-polmethacrylate copolymer, poly(methacrylamide-co-methacrylate)-gellan gum copolymer, thiolated gellan, acrylated poloxamine, poly(N-isopropylacrylamide), poly(phosphazene), collagen-(poly(glycolic acid)) copolymer, (glycosaminoglycan)-(polypeptide) polymer, (ulvan)-(polyisopropylacrylamide) copolymer, a mixture of poloxamers, a mixture of hyaluronic acid and (polycaprolactone-(polyethylene glycol)-polycaprolactone), and mixtures thereof;
ii. a nucleo-functional polymer selected from a N—O carboxymethyl chitosan, (poloxamer)-(chondroitan sulfate)-(polyethylene glycol), polyethylene glycol, (hyaluronic acid)-(polygalacturonic acid) copolymer, (hyaluronic acid)-(gelatin)-(polyethylene glycol) polymer, (hyaluronic acid)-(collagen)-(sericin) polymer, (hyaluronic acid)-dextran copolymer, star polyethylene glycol, (star polyethylene glycol)-dextran copolymer, lysine-functionalized polyethylene glycol, (polyethylene glycol)-(dendritic lysine) polymer, polyethylene glycol-polylysine copolymer, thioloated gellan, acylated-sulfobetaine-starch, acrylated poloxamine, polyamidoamine dendrimer, (polyamidoamine dendrimer)-dextran copolymer, chitosan-dextran copolymer, chitosan-alginate copolymer, (carboxymethyl chitosan)-(carboxymethyl cellulose) copolymer, hyaluronic acid, tetra-succinimidyl substituted polyethylene glycol, tetra-thiol-substituted polyethylene glycol, and mixtures thereof;
iii. an electro-functional polymer selected from a (polyethylene glycol)-(dendritic thioester) polymer, acrylated four-arm polymer containing (poly(p-phenylene oxide))-(polyethylene glycol)-(poly(p-phenylene oxide)), poly(methacrylamide-co-methacrylate)-gellan gum copolymer, chitosan-polylysine copolymer, hyaluronic acid, and mixtures thereof;
iv. a pH-sensitive polymer selected from (polyethylene glycol)-polyaspartylhydrazide copolymer, chitosan-alginate copolymer, chitosan-(gellan gum) copolymer, and mixtures thereof;
v. an ion-sensitive polymer selected from an alginate-chitosan-genipin polymer, chitosan-alginate copolymer, chitosan-(gellan gum) copolymer, gellan gum-kappa carrageenan copolymer, and mixtures thereof;
vi. a photo-sensitive polymer selected from a (polyethylene glycol)-lactide, (polyethylene glycol)-fibrinogen polymer, acrylate-(polyethylene glycolyl)-acrylate, alginate, gelatin, pHEMA-co-APMA-polyamidoamine, poly(6-aminohexyl propylene phosphate), carboxymethyl chitan, hyaluronic acid, and mixtures thereof;
vii. an enzyme-reactive polymer selected from a (polylysine)-(polyethylene glycol)-tyramine polymer, gelatin, pullulan, poly(phenylene oxide)-polyethylene glycol copolymer, gelatin-chitosan copolymer, and mixtures thereof;
viii. a pressure-sensitive polymer selected from (polyethylene glycol)-dihydroxyacetone;
ix. free-radical sensitive polymer selected from a betaine-containing polymer; and
x. a polymer selected from a (carboxymethylchitosan)-(oxidized alginate) copolymer, hyaluronic acid, (hyaluronic acid)-(crosslinked alginate) copolymer, (vinyl phosphonic acid)-acrylamide polymer, (poly(vinyl alcohol))-(carboxymethyl cellulose) copolymer, and mixtures thereof; and
xi. mixtures thereof; and
   (b) curing the biocompatible polymer to form a hydrogel in the vitreous cavity.

In certain embodiments, the curing comprises administering a curing agent to the vitreous cavity of an eye of the subject to facilitate curing of the biocompatible polymer. In certain embodiments, the biocompatible polymer is exposed to a curing agent prior to administering the biocompatible polymer to the vitreous cavity of the eye of the subject. In certain embodiments, the biocompatible polymer and a curing agent are administered concurrently to the vitreous cavity of the eye of the subject. In certain embodiments, the biocompatible polymer and an curing agent are administered concurrently to the eye of the subject in an amount effective to support the retinal tissue, such as an amount that upon formation of the hydrogel, the hydrogel contacts the retinal tissue.

The method can be further characterized by, for example, the identity of the subject. In certain embodiments, subject has a physical discontinuity in the retinal tissue. In certain embodiments, the physical discontinuity is a tear in the retinal tissue, a break in the retinal tissue, or a hole in the retinal tissue. In other embodiments, the subject has undergone surgery for a macular hole, has undergone surgery to remove at least a portion of a epiretinal membrane, or has undergone a vitrectomy for vitreomacular traction. In other embodiments, the subject has a detachment of at least a portion of the retinal tissue. The retinal detachment may be, for example, a rhegmatogenous retinal detachment. Alternatively, the retinal detachment may be tractional retinal detachment or serous retinal detachment.

In certain embodiments, the nucleo-functional polymer and an electro-functional polymer are administered to the eye of the subject in an amount effective to support the retinal tissue, such as an amount that upon formation of the hydrogel, the hydrogel contacts the retinal tissue.

In certain embodiments, the nucleo-functional polymer and the electro-functional polymer are administered separately to the vitreous cavity of the eye of the subject. In certain embodiments, the electro-functional polymer is administered as a liquid pharmaceutical formulation containing an aqueous pharmaceutically acceptable carrier to the vitreous cavity of the eye of the subject.

In certain embodiments, the method can also be further characterized by, for example, the identity of the nucleo-functional polymer, the identity of the electro-functional polymer, the identity of the poly(ethylene glycol) polymer, physical characteristics of the hydrogel formed, and/or other features described herein below.

In certain embodiments, the method can also be further characterized by, for example, the identity of the biocompatible polymer, the identity of the curing agent, physical characteristics of the hydrogel formed, and/or other features described herein below.

### Third Embodiment—Treating a Subject with a Retinal Detachment

Another aspect of the invention provides a method of treating a subject with a retinal detachment, the method comprising: (a) administering to the vitreous cavity of an eye of the subject with a detachment of at least a portion of retinal tissue an effective amount of (i) an electro-functional polymer and (ii) an ocular formulation comprising a nucleo-functional polymer, a poly(ethylene glycol) polymer, and an aqueous pharmaceutically acceptable carrier; and (b) allowing the nucleo-functional polymer and the electro-functional polymer to react to form a hydrogel in the vitreous cavity; wherein the hydrogel supports the retinal tissue during reattachment of the portion of the retinal tissue; wherein the nucleo-functional polymer is a biocompatible polyalkylene polymer substituted by (i) a plurality of —OH groups, (ii) a plurality of thio-functional groups —R¹—SH wherein R¹ is an ester-containing linker, and (iii) optionally one or more —OC(O)—(C₁-C₆ alkyl) groups; and the electro-functional polymer is a biocompatible polymer containing at least one thiol-reactive group. In certain embodiments, the invention provides a method of treating a subject with a retinal detachment, the method comprising: (a) administering a nucleo-functional polymer and an electro-functional polymer to the vitreous cavity of an eye of the subject with a detachment of at least a portion of retinal tissue; and (b) allowing the nucleo-functional polymer and the electro-functional polymer to react to form a hydrogel in the vitreous cavity; wherein the hydrogel supports the retinal tissue during reattachment of the portion of the retinal tissue, the nucleo-functional polymer is a biocompatible polyalkylene polymer substituted by (i) a plurality of —OH groups, (ii) a plurality of thio-functional groups —R¹—SH, (iii) at least one polyethylene glycolyl group, and (iv) optionally one or more —OC(O)—(C₁-C₆ alkyl) groups; R¹ is an ester-containing linker, and the electro-functional polymer is a biocompatible polymer containing at least one thiol-reactive group.

In certain embodiments, the method comprises:

(a) administering to the vitreous cavity of an eye of the subject an effective amount of a biocompatible polymer selected from the group consisting of:
i. a thermosensitive polymer selected from a hydroxybutyl chitosan, carboxymethyl chitosan, chitosan-(D)-glucose phosphate, (chitosan)-(hydroxypropylmethyl cellulose)-(glycerin) polymer, chitosan-(beta-gly cerophosphate)-hydroxyethyl cellulose polymer, (hyaluronic acid)-(hyperbranched polyethylene glycol) copolymer, poloxamer, (poloxamer)-(chondroitan sulfate)-(polyethylene glycol) polymer, (poly(lactic acid))-(poloxamer)-(poly(lactic acid) polymer, (polyethylene glycol)-polyalanine copolymer, (polyethylene glycol)-(poly caprolactone)-(polyethylene glycol) polymer, (polyethylene glycol)-(polyester urethane) copolymer, [poly(beta-benzyl L-aspartate)]-(polyethylene glycol)-[poly(beta-benzyl L-aspartate)], polycaprolactone-(polyethylene glycol)-polycaprolactone polymer, poly(lactic-co-glycolic acid)-(polyethylene glycol)-(poly(lactic-co-glycolic acid)), polymethacrylamide-polmethacrylate copolymer, poly(methacrylamide-co-methacrylate)-gellan gum copolymer, thiolated gellan, acrylated poloxamine, poly(N-isopropylacrylamide), poly(phosphazene), collagen-(poly(glycolic acid)) copolymer, (glycosaminoglycan)-(polypeptide) polymer, (ulvan)-(polyisopropylacrylamide) copolymer, a mixture of poloxamers, a mixture of hyaluronic acid and (polycaprolactone-(polyethylene glycol)-polycaprolactone), and mixtures thereof;
ii. a nucleo-functional polymer selected from a N—O carboxymethyl chitosan, (poloxamer)-(chondroitan sulfate)-(polyethylene glycol) polymer, polyethylene glycol, (hyaluronic acid)-(polygalacturonic acid) copolymer, (hyaluronic acid)-(gelatin)-(polyethylene glycol) polymer, (hyaluronic acid)-(collagen)-(sericin) polymer, (hyaluronic acid)-dextran copolymer, star polyethylene glycol, (star polyethylene glycol)-dextran copolymer, lysine-functionalized polyethylene glycol, (polyethylene glycol)-(dendritic lysine) polymer, polyethylene glycol-polylysine copolymer, thioloated gellan, acylated-sulfobetaine-starch, acrylated poloxamine, polyamidoamine dendrimer, (polyamidoamine dendrimer)-dextran copolymer, chitosan-dextran copolymer, chitosan-alginate copolymer, (carboxymethyl chitosan)-(carboxymethyl cellulose) copolymer, hyaluronic acid, tetra-succinimidyl substituted polyethylene glycol, tetra-thiol-substituted polyethylene glycol, and mixtures thereof;
iii. an electro-functional polymer selected from a (polyethylene glycol)-(dendritic thioester) polymer, acrylated four-arm polymer containing (poly(p-phenylene oxide))-(polyethylene glycol)-(poly(p-phenylene oxide)), poly(methacrylamide-co-methacrylate)-gellan gum copolymer, chitosan-polylysine copolymer, hyaluronic acid, and mixtures thereof;
iv. a pH-sensitive polymer selected from (polyethylene glycol)-polyaspartylhydrazide copolymer, chitosan-alginate copolymer, chitosan-(gellan gum) copolymer, and mixtures thereof;
v. an ion-sensitive polymer selected from an alginate-chitosan-genipin polymer, chitosan-alginate copolymer, chitosan-(gellan gum) copolymer, gellan gum-kappa carrageenan copolymer, and mixtures thereof;
vi. a photo-sensitive polymer selected from a (polyethylene glycol)-lactide, (polyethylene glycol)-fibrinogen polymer, acrylate-(polyethylene glycolyl)-acrylate, alginate, gelatin, pHEMA-co-APMA-polyamidoamine, poly(6-aminohexyl propylene phosphate), carboxymethyl chitan, hyaluronic acid, and mixtures thereof;
vii. an enzyme-reactive polymer selected from a (polylysine)-(polyethylene glycol)-tyramine polymer, gelatin, pullulan, poly(phenylene oxide)-polyethylene glycol copolymer, gelatin-chitosan copolymer, and mixtures thereof;
viii. a pressure-sensitive polymer selected from (polyethylene glycol)-dihydroxyacetone;
ix. free-radical sensitive polymer selected from a betaine-containing polymer; and
x. a polymer selected from a (carboxymethylchitosan)-(oxidized alginate) copolymer, hyaluronic acid, (hyaluronic acid)-(crosslinked alginate) copolymer, (vinyl phosphonic acid)-acrylamide polymer, (poly(vinyl alcohol))-(carboxymethyl cellulose) copolymer, and mixtures thereof, and
xi. mixtures thereof, and
   (b) curing the biocompatible polymer to form a hydrogel in the vitreous cavity.

In certain embodiments, the curing comprises administering a curing agent to the vitreous cavity of an eye of the subject to facilitate curing of the biocompatible polymer. In certain embodiments, the biocompatible polymer is exposed to a curing agent prior to administering the biocompatible polymer to the vitreous cavity of the eye of the subject. In certain embodiments, the biocompatible polymer and a curing agent are administered concurrently to the vitreous cavity of the eye of the subject.

The method can be further characterized by, for example, the nature of the retinal detachment. In certain embodiments, the retinal detachment is a rhegmatogenous retinal detachment. In other embodiments, the subject has tractional retinal detachment or serous retinal detachment.

In certain embodiments, the nucleo-functional polymer and an electro-functional polymer are administered to the eye of the subject in an amount effective to support the retinal tissue, thereby facilitating treatment of the retinal detachment.

In certain embodiments, the nucleo-functional polymer and the electro-functional polymer are administered separately to the vitreous cavity of the eye of the subject. In certain embodiments, the electro-functional polymer is administered as a liquid pharmaceutical formulation containing an aqueous pharmaceutically acceptable carrier to the vitreous cavity of the eye of the subject.

In certain embodiments, the biocompatible polymer is administered to the eye of the subject in an amount effective to support the retinal tissue, thereby facilitating treatment of the retinal detachment.

The method can also be further characterized by, for example, the identity of the nucleo-functional polymer, the identity of the electro-functional polymer, the identity of the poly(ethylene glycol) polymer, the identity of the biocompatible polymer, the presence and identity of a curing agent, physical characteristics of the hydrogel formed, and/or other features described herein below.

### Fourth Embodiment—Treating Hypotony

Another aspect of the invention provides a method of treating a subject with low pressure in the eye (i.e., hypotony), the method comprising: (a) administering to the vitreous cavity of an eye of the subject an effective amount of (i) an electro-functional polymer and (ii) an ocular formulation comprising a nucleo-functional polymer, a poly(ethylene glycol) polymer, and an aqueous pharmaceutically acceptable carrier; and (b) allowing the nucleo-functional polymer and the electro-functional polymer to react to form a hydrogel in the vitreous cavity; to thereby treat the subject with low pressure in the eye, wherein the nucleo-functional polymer is a biocompatible polyalkylene polymer substituted by (i) a plurality of —OH groups, (ii) a plurality of thio-functional groups —R¹—SH wherein R¹ is an ester-containing linker, and (iii) optionally one or more —OC(O)—(C₁-C₆ alkyl) groups; and wherein the electro-functional polymer is a biocompatible polymer containing at least one thiol-reactive group. In certain embodiments, the method causes an increase in pressure of at least about 1 mmHg, 2 mmHg, 5 mmHg, 7 mmHg, or 10 mmHg in the eye of the subject. In some embodiments, the invention provides a method of treating a subject with low pressure in the eye (i.e., hypotony), the method comprising: (a) administering an effective amount of a nucleo-functional polymer and an electro-functional polymer to the vitreous cavity of an eye of the subject; and (b) allowing the nucleo-functional polymer and the electro-functional polymer to react to form a hydrogel in the vitreous cavity; to thereby treat the subject with low pressure in the eye, wherein the nucleo-functional polymer is a biocompatible polyalkylene polymer substituted by (i) a plurality of —OH groups, (ii) a plurality of thio-functional groups —R¹—SH, (iii) at least one polyethylene glycolyl group, and (iv) optionally one or more —OC(O)—(C₁-C₆ alkyl) groups; R¹ is an ester-containing linker, and the electro-functional polymer is a biocompatible polymer containing at least one thiol-reactive group. In certain embodiments, the method causes an increase in pressure of at least about 1 mmHg, 2 mmHg, 5 mmHg, 7 mmHg, or 10 mmHg in the eye of the subject.

In certain embodiments, the invention provides a method of treating a subject with low pressure in the eye (i.e., hypotony), the method comprising: (a) administering an effective amount of a biocompatible polymer described herein to the vitreous cavity of an eye of the subject; and (b) curing the biocompatible polymer to form a hydrogel in the vitreous cavity; to thereby treat the subject with low pressure in the eye. In certain embodiments, the method causes an increase in pressure of at least about 1 mmHg, 2 mmHg, 5 mmHg, 7 mmHg, or 10 mmHg in the eye of the subject.

In certain embodiments, the curing comprises administering a curing agent to the vitreous cavity of an eye of the subject to facilitate curing of the biocompatible polymer. In certain embodiments, the biocompatible polymer is exposed to a curing agent prior to administering the biocompatible polymer to the vitreous cavity of the eye of the subject. In certain embodiments, the biocompatible polymer and a curing agent are administered concurrently to the vitreous cavity of the eye of the subject.

In certain embodiments, the subject suffers from a choroidal effusion (e.g., a serous choroidal effusion or hemorrhagic choroidal effusion).

The method can also be further characterized by, for example, the identity of the nucleo-functional polymer, the identity of the electro-functional polymer, the identity of the poly(ethylene glycol) polymer, the identity of the biocompatible polymer, the presence and identity of a curing agent, physical characteristics of the hydrogel formed, and/or other features described herein below.

### Fifth Embodiment—Treating Choroidal Effusion

Another aspect of the invention provides a method of treating a choroidal effusion, the method comprising: (a) administering an effective amount of (i) an electro-functional polymer and (ii) an ocular formulation comprising a nucleo-functional polymer, a poly(ethylene glycol) polymer, and an aqueous pharmaceutically acceptable carrier, to an eye of the subject having a choroidal effusion; and (b) allowing the nucleo-functional polymer and the electro-functional polymer to react to form a hydrogel; to thereby treat the choroidal effusion, wherein the nucleo-functional polymer is a biocompatible polyalkylene polymer substituted by (i) a plurality of —OH groups, (ii) a plurality of thio-functional groups —R¹—SH wherein R¹ is an ester-containing linker, and (iii) optionally one or more —OC(O)—(C₁-C₆ alkyl) groups; and wherein the electro-functional polymer is a biocompatible polymer containing at least one thiol-reactive group. In some embodiments, the invention provides a method of treating a choroidal effusion, the method comprising: (a) administering an effective amount of a nucleo-functional polymer and an electro-functional polymer to an eye of the subject having a choroidal effusion; and (b) allowing the nucleo-functional polymer and the electro-functional polymer to react to form a hydrogel; to thereby treat the choroidal effusion, wherein the nucleo-functional polymer is a biocompatible polyalkylene polymer substituted by (i) a plurality of —OH groups, (ii) a plurality of thio-functional groups —R¹—SH, (iii) at least one polyethylene glycolyl group, and (iv) optionally one or more —OC(O)—(C₁-C₆ alkyl) groups; R¹ is an ester-containing linker, and the electro-functional polymer is a biocompatible polymer containing at least one thiol-reactive group.

In certain embodiments, the invention provides a method of treating a choroidal effusion, the method comprising: (a) administering an effective amount of a biocompatible polymer to an eye of the subject having a choroidal effusion; and (b) curing the biocompatible polymer to form a hydrogel; to thereby treat the choroidal effusion.

In certain embodiments, the curing comprises administering a curing agent to the vitreous cavity of an eye of the subject to facilitate curing of the biocompatible polymer. In certain embodiments, the biocompatible polymer is exposed to a curing agent prior to administering the biocompatible polymer to the vitreous cavity of the eye of the subject. In certain embodiments, the biocompatible polymer and a curing agent are administered concurrently to the vitreous cavity of the eye of the subject.

In certain embodiments, the choroidal effusion is a serous choroidal effusion or hemorrhagic choroidal effusion.

In certain embodiments, the method causes an increase in pressure of at least about 1 mmHg, 2 mmHg, 5 mmHg, 7 mmHg, or 10 mmHg in the eye of the subject.

The method can also be further characterized by, for example, the identity of the nucleo-functional polymer, the identity of the electro-functional polymer, the identity of the poly(ethylene glycol) polymer, the identity of the biocompatible polymer, the presence and identity of a curing agent, physical characteristics of the hydrogel formed, and/or other features described herein below.

### Sixth Embodiment—Improving Visual Performance

Another aspect of the invention provides a method of improving visual performance in a patient suffering from a retinal detachment, the method comprising: (a) administering to the vitreous cavity of an eye of the subject an effective amount of (i) an electro-functional polymer and (ii) an ocular formulation comprising a nucleo-functional polymer, a poly(ethylene glycol) polymer, and an aqueous pharmaceutically acceptable carrier; and (b) allowing the nucleo-functional polymer and the electro-functional polymer to react to form a hydrogel in the vitreous cavity; wherein the nucleo-functional polymer is a biocompatible polyalkylene polymer substituted by (i) a plurality of —OH groups, (ii) a plurality of thio-functional groups —R¹—SH wherein R¹ is an ester-containing linker, and (iii) optionally one or more —OC(O)—(C₁-C₆ alkyl) groups; and wherein the electro-functional polymer is a biocompatible polymer containing at least one thiol-reactive group. In certain embodiments, the invention provides a method of improving visual performance in a patient suffering from a retinal detachment, the method comprising: (a) administering to the vitreous cavity of an eye of the subject an effective amount of nucleo-functional polymer and an electro-functional polymer; and (b) allowing the nucleo-functional polymer and the electro-functional polymer to react to form a hydrogel in the vitreous cavity; wherein the nucleo-functional polymer is a biocompatible polyalkylene polymer substituted by (i) a plurality of —OH groups, (ii) a plurality of thio-functional groups —R¹—SH, (iii) at least one polyethylene glycolyl group, and (iv) optionally one or more —OC(O)—(C₁-C₆ alkyl) groups; R¹ is an ester-containing linker, and the electro-functional polymer is a biocompatible polymer containing at least one thiol-reactive group.

In certain embodiments, the invention provides a method of improving visual performance in a subject suffering from a retinal detachment, the method comprising: (a) administering to the vitreous cavity of an eye of the subject an effective amount of biocompatible polymer described herein; and (b) curing the biocompatible polymer to form a hydrogel in the vitreous cavity.

In certain embodiments, the curing comprises administering a curing agent to the vitreous cavity of an eye of the subject to facilitate curing of the biocompatible polymer. In certain embodiments, the biocompatible polymer is exposed to a curing agent prior to administering the biocompatible polymer to the vitreous cavity of the eye of the subject. In certain embodiments, the biocompatible polymer and a curing agent are administered concurrently to the vitreous cavity of the eye of the subject.

The method can be further characterized by, for example, the identity of the subject. In certain embodiments, the subject may have suffered from a retinal detachment that is a rhegmatogenous retinal detachment. Alternatively, the retinal detachment may be tractional retinal detachment or serous retinal detachment.

The nucleo-functional polymer and an electro-functional polymer are administered to the eye of the subject in an amount effective to support the retinal tissue, such as an amount that upon formation of the hydrogel, the hydrogel contacts the retinal tissue.

Visual performance pertains to the patient's overall vision quality and includes a patient's ability to see clearly, as well as ability to distinguish between an object and its background. One aspect of visual performance is visual acuity, which is a measure of a patient's ability to see clearly. Visual acuity can be assessed, for example, by using conventional “eye charts” in which visual acuity is evaluated by the ability to discern letters of a certain size, with five letters of a given size present on each line (see, e.g., the “ETDRS” eye chart described in the Murphy, R.P., CURRENT TECHNIQUES IN OPHTHALMIC LASER SURGERY, 3^{rd} Ed., edited by L. D. Singerman, and G. Cascas, Butterworth Heinemann, 2000). Evaluation of visual acuity may also be achieved by measuring reading speed and reading time. Visual acuity may be measured to evaluate whether administration of a necrosis inhibitor and/or an apoptosis inhibitor to the affected eye preserves or permits improvement of visual acuity (e.g., to 20/40 vision or to 20/20 vision). In certain embodiments, a Snellen chart can be used to measure a patient's visual acuity, and the measurement can be taken under conditions that test low-contrast visual acuity or under conditions that test high-contrast visual acuity. Also, the visual acuity measurement can be taken under scotopic conditions, mesopic conditions, and/or photopic conditions.

Another aspect of visual performance is contrast sensitivity, which is a measure of the patient's ability to distinguish between an object and its background. The contrast sensitivity can be measured under various light conditions, including, for example, photopic conditions, mesopic conditions, and scotopic conditions. In certain embodiments, the contrast sensitivity is measured under mesopic conditions.

In certain embodiments, the improvement in visual performance provided by the method is improved visual acuity. In certain embodiments, the improvement in visual performance provided by the method is improved visual acuity under scotopic conditions. In certain embodiments, the improvement in visual performance provided by the method is improved visual acuity under mesopic conditions. In certain embodiments, the improvement in visual performance provided by the method is improved visual acuity under photopic conditions. In certain embodiments, the improvement in visual acuity is a two-line improvement in the patient's vision as measured using the Snellen chart. In certain other embodiments, the improvement in visual acuity is a one-line improvement in the patient's vision as measured using the Snellen chart.

In certain embodiments, the improvement in visual performance provided by the method is improved contrast sensitivity. The improvement in contrast sensitivity can be measured under various light conditions, such as photopic conditions, mesopic conditions, and scotopic conditions. In certain embodiments, the improvement in visual performance provided by the method is improved contrast sensitivity under photopic conditions. In certain embodiments, the improvement in visual performance provided by the method is improved contrast sensitivity under mesopic conditions. In certain embodiments, the improvement in visual performance provided by the method is improved contrast sensitivity under scotopic conditions.

Results achieved by the methods can be characterized according to the patient's improvement in contrast sensitivity. For example, in certain embodiments, the improvement in contrast sensitivity is at least a 10%, 20%, 30%, 50%, 60%, 70%, 80%, 90%, or 100% improvement measured under mesopic conditions using an art-recognized test, such as a Holladay Automated Contrast Sensitivity System. In certain embodiments, the improvement in contrast sensitivity is at least a 10%, 20%, 30%, 50%, 60%, 70%, 80%, 90%, or 100% improvement measured under photopic conditions using an art-recognized test, such as a Holladay Automated Contrast Sensitivity System. In certain other embodiments, the improvement in contrast sensitivity is at least a 10%, 20%, 30%, 50%, 60%, 70%, 80%, 90%, or 100% improvement measured under mesopic conditions or scotopic conditions using an art-recognized test, such a Holladay Automated Contrast Sensitivity System.

Visual performance may also be measured by determining whether there is an increase in the thickness of the macula (e.g., macula thickness is 15% thicker than, 35% thicker than, 50% thicker than, 60% thicker than, 70% thicker than, or 80% thicker than a macula without the treatment as measured by optical coherence tomography (OCT); an improvement of the photoreceptor cell layer or its subdivisions as seen in the OCT; an improvement of visual field (e.g., by at least 10% in the mean standard deviation on the Humphrey Visual Field Test; an improvement of an electroretinograph (ERG), a measurement of the electrical response of the retina to light stimulation, (e.g., to increase ERG amplitude by at least 15%); and or preservation or improvement of multifocal ERG, which evaluates the response of the retina to multifocal stimulation and allows characterization of the function of a limited area of the retina.

Visual performance may also be measured by electrooculography (EOG), which is a technique for measuring the resting potential of the retina. EOG is particularly useful for the assessment of RPE function. EOG may be used to evaluate whether administration of a necrosis inhibitor and/or an apoptosis inhibitor to the retina of the affected eye preserves or permits improvement in, for example, the Arden ratio (e.g., an increase in Arden ratio of at least 10%).

Visual performance may also be assessed through fundus autofluorescence (AF) imaging, which is a clinical tool that allows evaluation of the interaction between photoreceptor cells and the RPE. For example, increased fundus AF or decreased fundus AF has been shown to occur in AMD and other ocular disorders. Fundus AF imaging may be used to evaluate whether administration of a necrosis inhibitor and/or an apoptosis inhibitor to the retina of the affected eye slows disease progression.

Visual performance may also be assessed by microperimetry, which monitors retinal visual function against retinal thickness or structure and the condition of the subject's fixation over time. Microperimetry may be used to assess whether administration of a necrosis inhibitor and/or an apoptosis inhibitor to the retina of the affected eye preserves or permits improvement in retinal sensitivity and fixation.

The method can also be further characterized by, for example, the identity of the nucleo-functional polymer, the identity of the electro-functional polymer, the identity of the poly(ethylene glycol) polymer, the identity of the biocompatible polymer, the presence and identity of a curing agent, physical characteristics of the hydrogel formed, and/or other features described herein below.

### Seventh Embodiment—Supporting Tissue in or Adjacent to the Anterior Chamber of the Eye

Another aspect of the invention provides a method of supporting tissue in or adjacent to the anterior chamber of the eye of a subject, the method comprising: (a) administering an effective amount of (i) an electro-functional polymer and (ii) an ocular formulation comprising a nucleo-functional polymer, a poly(ethylene glycol) polymer, and an aqueous pharmaceutically acceptable carrier, to the anterior chamber of an eye of the subject; and (b) allowing the nucleo-functional polymer and the electro-functional polymer to react to form a hydrogel in the anterior chamber; wherein the nucleo-functional polymer is a biocompatible polyalkylene polymer substituted by (i) a plurality of —OH groups, (ii) a plurality of thio-functional groups —R¹—SH wherein R¹ is an ester-containing linker, and (iii) optionally one or more —OC(O)—(C₁-C₆ alkyl) groups; and wherein the electro-functional polymer is a biocompatible polymer containing at least one thiol-reactive group. In certain embodiments, the invention provides a method of supporting tissue in or adjacent to the anterior chamber of the eye of a subject, the method comprising: (a) administering an effective amount of a nucleo-functional polymer and an electro-functional polymer to the anterior chamber of an eye of the subject; and (b) allowing the nucleo-functional polymer and the electro-functional polymer to react to form a hydrogel in the anterior chamber; wherein the nucleo-functional polymer is a biocompatible polyalkylene polymer substituted by (i) a plurality of —OH groups, (ii) a plurality of thio-functional groups —R¹—SH, (iii) at least one polyethylene glycolyl group, and (iv) optionally one or more —OC(O)—(C₁-C₆ alkyl) groups; R¹ is an ester-containing linker, and the electro-functional polymer is a biocompatible polymer containing at least one thiol-reactive group. In some embodiments, the invention provides a method of supporting tissue in or adjacent to the anterior chamber of the eye of a subject, the method comprising: (a) administering an effective amount of a biocompatible polymer described herein to the anterior chamber of an eye of the subject; and (b) curing the biocompatible polymer to form a hydrogel in the anterior chamber. In certain embodiments, the method supports a graft in the anterior chamber of the eye. The hydrogel achieves supporting tissue in or adjacent to the anterior chamber of the eye by coming into contact with such tissue and optionally exerting a force (e.g., 0.1, 0.5, 1.0, or 2.0 N) against such tissue.

The method can also be further characterized by, for example, the identity of the nucleo-functional polymer, the identity of the electro-functional polymer, the identity of the poly(ethylene glycol) polymer, the identity of the biocompatible polymer, the presence and identity of a curing agent, physical characteristics of the hydrogel formed, and/or other features described herein below.

### Eighth Embodiment—Maintaining or Expanding a Nasolacrimal Duct

Another aspect of the invention provides a method of maintaining or expanding a nasolacrimal duct in a subject, the method comprising: (a) administering an effective amount of (i) an electro-functional polymer and (ii) an ocular formulation comprising a nucleo-functional polymer, a poly(ethylene glycol) polymer, and an aqueous pharmaceutically acceptable carrier, to a nasolacrimal duct in a subject; and (b) allowing the nucleo-functional polymer and the electro-functional polymer to react to form a hydrogel in the nasolacrimal duct; wherein the nucleo-functional polymer is a biocompatible polyalkylene polymer substituted by (i) a plurality of —OH groups, (ii) a plurality of thio-functional groups —R¹—SH wherein R¹ is an ester-containing linker, and (iii) optionally one or more —OC(O)—(C₁-C₆ alkyl) groups; and wherein the electro-functional polymer is a biocompatible polymer containing at least one thiol-reactive group. In certain embodiments, the invention provides a method of maintaining or expanding a nasolacrimal duct in a subject, the method comprising: (a) administering an effective amount of a nucleo-functional polymer and an electro-functional polymer to a nasolacrimal duct in a subject; and (b) allowing the nucleo-functional polymer and the electro-functional polymer to react to form a hydrogel in the nasolacrimal duct; wherein the nucleo-functional polymer is a biocompatible polyalkylene polymer substituted by (i) a plurality of —OH groups, (ii) a plurality of thio-functional groups —R¹—SH, (iii) at least one polyethylene glycolyl group, and (iv) optionally one or more —OC(O)—(C₁-C₆ alkyl) groups; R¹ is an ester-containing linker, and the electro-functional polymer is a biocompatible polymer containing at least one thiol-reactive group. In some embodiments, the invention provides a method of maintaining or expanding a nasolacrimal duct in a subject, the method comprising: (a) administering an effective amount of a biocompatible polymer to a nasolacrimal duct in a subject; and (b) curing the biocompatible polymer to form a hydrogel in the nasolacrimal duct. In certain embodiments, the hydrogel achieves maintaining or expanding a nasolacrimal duct by coming into contact with such tissue and optionally exerting a force (e.g., 0.1, 0.5, 1.0, or 2.0 N) against such tissue.

In certain embodiments, the method further comprises administering a curing agent to the nasolacrimal duct of the subject to facilitate curing of the biocompatible polymer. In certain embodiments, the biocompatible polymer is exposed to a curing agent prior to administering the biocompatible polymer to the nasolacrimal duct of the subject. In certain embodiments, the biocompatible polymer and a curing agent are administered concurrently to the nasolacrimal duct of the subject.

The method can also be further characterized by, for example, the identity of the nucleo-functional polymer, the identity of the electro-functional polymer, the identity of the poly(ethylene glycol) polymer, the identity of the biocompatible polymer, the presence and identity of a curing agent, physical characteristics of the hydrogel formed, and/or other features described herein below.

### Injectable, Ocular Formulation for Forming a Hydrogel

Another aspect of the invention provides an injectable, ocular formulation for forming a hydrogel in the eye of a subject, the formulation comprising: (a) a nucleo-functional polymer that is a biocompatible polyalkylene polymer substituted by (i) a plurality of —OH groups, (ii) a plurality of thio-functional groups —R¹—SH wherein R¹ is an ester-containing linker, and (iii) optionally one or more —OC(O)—(C₁-C₆ alkyl) groups; (b) a poly(ethylene glycol) polymer; and (c) an aqueous pharmaceutically acceptable carrier for administration to the eye of a subject. In certain embodiments, the invention provides an injectable, ocular formulation for forming a hydrogel in the eye of a subject, the formulation comprising: (a) a nucleo-functional polymer that is a biocompatible polyalkylene polymer substituted by (i) a plurality of —OH groups, (ii) a plurality of thio-functional groups —R¹—SH, (iii) at least one polyethylene glycolyl group, and (iv) optionally one or more —OC(O)—(C₁-C₆ alkyl) groups; R¹ is an ester-containing linker; (b) an electro-functional polymer that is a biocompatible polymer containing at least one thiol-reactive group; and (c) a liquid pharmaceutically acceptable carrier for administration to the eye of a subject. In some embodiments, the invention provides an injectable, ocular formulation for forming a hydrogel in the eye of a subject, the formulation comprising: (a) a biocompatible polymer described herein and (b) a liquid pharmaceutically acceptable carrier for administration to the eye of a subject. The formulation can be further characterized by, for example, the identity of the nucleo-functional polymer, the identity of the electro-functional polymer, the identity of the poly(ethylene glycol) polymer, the identity of the biocompatible polymer, the presence and identity of a curing agent, physical characteristics of the hydrogel formed, and/or other features described herein below

### General Features of the Methods and Injectable Ocular Formulation

General features of the methods and injectable ocular formulation are described below.

### Features of the Hydrogel

The therapeutic methods and compositions for forming hydrogels can be further characterized according to features of the hydrogel. Exemplary features of the hydrogel include, for example, refractive index, transparency, density, gelation time, elastic modulus, viscosity (e.g., dynamic viscosity), biodegradation, and pressure generated by the hydrogel within the eye or other location into which the polymers for forming a hydrogel are inserted.

In certain embodiments, the hydrogel is formed by reaction of the nucleo-functional polymer and electro-functional polymer, and the subsequent update of water from the subject (e.g., the subject's eye). In the more specific embodiment of a thiolated poly(vinyl alcohol) polymer as the nucleo-functional polymer and a poly(ethylene glycol) (PEG) containing thiol-reactive groups as the electro-functional polymer, the hydrogel is formed by a cross-linking reaction of thiolated poly(vinyl alcohol) (TPVA) with poly(ethylene glycol) (PEG) containing thiol-reactive groups. The thiolated poly(vinyl alcohol) polymer can be prepared according to procedures described in the literature (see, for example, U.S. Patent Application Publication No. 2016/0009872, which is hereby incorporated by reference), whereby thiol groups are incorporated into poly(vinylalcohol) (PVA) by coupling thiol functionalities to the hydroxyl groups of the poly(vinyl alcohol), or through use of protected thiol functionalities with subsequent deprotection. In certain embodiments, the nucleo-functional polymer can be prepared by reacting (a) a biocompatible polyalkylene polymer substituted by (i) a plurality of —OH groups, (ii) at least one polyethylene glycolyl group, and (iii) optionally one or more —OC(O)—(C₁-C₆ alkyl) groups with (b) HOC(O)—(C₁-C₆ alkylene)-SH, under conditions that promote reaction of a hydroxyl group with HOC(O)—(C₁-C₆ alkylene)-SH to form an ester bond, to thereby form the nucleo-functional polymer that is a biocompatible polyalkylene polymer substituted by (i) a plurality of —OH groups, (ii) a plurality of thio-functional groups —R¹—SH, (iii) at least one polyethylene glycolyl group, and (iv) optionally one or more —OC(O)—(C₁-C₆ alkyl) groups; where —R¹—SH is —OC(O)—(C₁-C₆ alkylene)-SH. An exemplary biocompatible polyalkylene polymer substituted by (i) a plurality of —OH groups, and (ii) at least one polyethylene glycolyl group contemplated for use is the polyvinyl alcohol-polyethylene glycol graft-copolymer having a weight-average molecular weight of about 45,000 g/mol sold by BASF under the tradename KOLLICOAT® IR. Another exemplary biocompatible polyalkylene polymer substituted by (i) a plurality of —OH groups, (ii) at least one polyethylene glycolyl group, and (iii) a plurality of —OC(O)—(C₁-C₆ alkyl) groups contemplated for use is a polyethylene glycol substituted polyvinyl alcohol polymer having a saponification degree of 86.5 to 89.5 mole percent and a weight-average molecular weight of about 50,000 g/mol sold by Gohsenol under product number WO-320R. Another exemplary biocompatible polyalkylene polymer substituted by (i) a plurality of —OH groups, (ii) at least one polyethylene glycolyl group, and (iii) a plurality of —OC(O)—(C₁-C₆ alkyl) groups contemplated for use is a polyethylene glycol substituted polyvinyl alcohol polymer having a saponification degree of at least 98.5 mole percent and a weight-average molecular weight of about 50,000 g/mol sold by Gohsenol under product number WO-320N. Certain poly(ethylene glycol) polymers containing thiol-reactive groups (e.g., an acrylate, methacrylate, maleimidyl, or N-hydroxysuccinimidyl) have been described in the literature (see, for example, U.S. Patent Application Publication No. 2016/0009872).

Crosslinking of the thiolated poly(vinyl alcohol) or the nucleo-functional polymer and the poly(ethylene glycol) containing thiol-reactive groups occurs through a Michael addition, without formation of by-products and does not require use of toxic initiators or a UV source. Further, there is no medically significant release of heat during the cross-linking reaction. Moreover, a freeze-thaw process is not required, as is commonly used to form poly(vinyl alcohol) hydrogels. Therefore, the nucleo-functional polymer and electro-functional polymer can be mixed easily in an operating room. Also, to the extent there are any unreacted nucleo-functional polymer and/or electro-functional polymer, the molecular weight of these components is desirably low enough that they will be readily cleared from the eye by natural processes.

In some embodiments, the hydrogel is formed by curing of the biocompatible polymer (which may be facilitated by exposing the biocompatible polymer to a curing agent), and the subsequent update of water from the subject (e.g., the subject's eye).

### Refractive Index

The therapeutic methods and compositions can be characterized according to the refractive index of hydrogel formed. For example, in certain embodiments, the hydrogel has a refractive index of greater than 1.0. In certain embodiments, the hydrogel has a refractive index in the range of from about 1.2 to about 1.5. In certain other embodiments, the hydrogel has a refractive index in the range of from about 1.3 to about 1.4. In certain other embodiments, the hydrogel has a refractive index in the range of from about 1.30 to about 1.35, or from about 1.31 to about 1.36. Methods and devices for measuring the refractive index are known in the art. For example, refractive index may be measured using an Atago Pocket Refractometer (PAL-BX/RI) using standard and known procedures.

### Transparency

The therapeutic methods and compositions can be characterized according to the transparency of the hydrogel formed. For example, in certain embodiments, the hydrogel has a transparency of at least 95% for light in the visible spectrum when measured through hydrogel having a thickness of 2 cm. In certain embodiments, the hydrogel has a transparency of at least 90%, 94%, or 98% for light in the visible spectrum when measured through hydrogel having a thickness of 2 cm.

### Density

The therapeutic methods and compositions can be characterized according to the density of the hydrogel formed. For example, in certain embodiments, the hydrogel has a density in the range of about 1 to about 1.5 g/mL. In certain other embodiments, the hydrogel has a density in the range of about 1 to about 1.2 g/mL, about 1.1 to about 1.3 g/mL, about 1.2 to about 1.3 g/mL, or about 1.3 to about 1.5 g/mL. In certain other embodiments, the hydrogel has a density in the range of about 1 to about 1.2 g/mL. In certain other embodiments, the hydrogel has a density in the range of about 1 to about 1.1 g/mL.

### Gelation Time

The therapeutic methods and compositions can be characterized according to the gelation time of the hydrogel (i.e., how long it takes for the hydrogel to form once the nucleo-functional polymer has been combined with the electro-functional polymer). Gelation time may also be referred to as cross-link time. For example, in certain embodiments, the hydrogel has a gelation time from about 1 minute to about 30 minutes after combining the nucleo-functional polymer and the electro-functional polymer. In certain embodiments, the hydrogel has a gelation time from about 5 minutes to about 30 minutes after combining the nucleo-functional polymer and the electro-functional polymer. In certain other embodiments, the hydrogel has a gelation time from about 5 minutes to about 20 minutes after combining the nucleo-functional polymer and the electro-functional polymer. In certain other embodiments, the hydrogel has a gelation time from about 5 minutes to about 10 minutes after combining the nucleo-functional polymer and the electro-functional polymer. In certain other embodiments, the hydrogel has a gelation time from about 1 minutes to about 5 minutes after combining the nucleo-functional polymer and the electro-functional polymer. In some embodiments, the hydrogel has a gelation time from about 2 minutes to about 5 minutes after combining the nucleo-functional polymer and the electro-functional polymer. In certain other embodiments, the hydrogel has a gelation time of less than about 1, 5, 10, 15, 20, 25, 30, 35, 40, 45, 50, 55 or 60 minutes. In some embodiments, the therapeutic methods and compositions can be characterized according to how long it takes for the hydrogel to form once the biocompatible polymer has been exposed to a curing agent. For example, in certain embodiments, the hydrogel has a gelation time from about 1 minute to about 30 minutes. In certain embodiments, the hydrogel has a gelation time from about 5 minutes to about 30 minutes. In certain other embodiments, the hydrogel has a gelation time from about 5 minutes to about 20 minutes. In certain other embodiments, the hydrogel has a gelation time from about 5 minutes to about 10 minutes. In certain other embodiments, the hydrogel has a gelation time of less than about 1, 5, 10, 15, 20, 25, 30, 35, 40, 45, 50, 55 or 60 minutes.

### Elastic Modulus

The therapeutic methods and compositions can be characterized according to the elastic modulus of the hydrogel formed. For example, in certain embodiments, the hydrogel has an elastic modulus in the range of from about 200 Pa to about 15 kPa at a temperature of 25° C. In certain embodiments, the hydrogel has an elastic modulus in the range of from about 600 Pa to about 7 kPa at a temperature of 25° C.

### Dynamic Viscosity

The therapeutic methods and compositions can be characterized according to the dynamic viscosity of the hydrogel formed. For example, in certain embodiments, the hydrogel has a dynamic viscosity in the range of about 20 to 60 cP at a temperature of 20° C.

### Biodegradation

The therapeutic methods and compositions can be characterized according whether the hydrogel is biodegradable. Accordingly, in certain embodiments, the hydrogel is biodegradable. A biodegradable hydrogel can be further characterized according to the rate at which the hydrogel undergoes biodegradation from the eye. In certain embodiments, the hydrogel undergoes complete biodegradation from the eye of the subject within about 7 days to about 30 days. In certain embodiments, the hydrogel undergoes complete biodegradation from the eye of the subject within about 1 week to about 4 weeks. In certain embodiments, the hydrogel undergoes complete biodegradation from the eye of the subject within about 2 weeks to about 8 weeks. In certain embodiments, the hydrogel undergoes complete biodegradation from the eye of the subject within about 3 weeks to about 5 weeks. In certain embodiments, the hydrogel undergoes complete biodegradation from the eye of the subject within about 4 months to about 6 months. In certain embodiments, the hydrogel undergoes complete biodegradation from the eye of the subject within about 3 days to about 7 days. In certain embodiments, the hydrogel undergoes complete biodegradation from the eye of the subject within 1, 2, 3, 4, 5, 6, or 7 days. In certain embodiments, the hydrogel undergoes complete biodegradation from the eye of the subject within 1, 2, 3, 4, 5, 6, 7, 8, 9, 10, 11, 12, 13, 14, 15, 16, 17, 18, 19, 20, 21, 22, 23, or 24 weeks. In certain embodiments, the hydrogel undergoes complete biodegradation from the eye of the subject within 1, 2, 3, 4, 5, 6, 7, 8, 9, 10, 11, 12, 13, 14, 15, 16, 17, 18, 19, 20, 21, 22, 23, or 24 months.

In certain embodiments, the hydrogel has a biodegradation half-life in the range of from about 4 days to about 20 days when disposed within the vitreous cavity of an eye. In certain embodiments, the hydrogel has a biodegradation half-life in the range of from about 1 month to about 2 months when disposed within the vitreous cavity of an eye. In certain embodiments, the hydrogel has a biodegradation half-life in the range of from about 1 week to about 3 weeks when disposed within the vitreous cavity of an eye. In certain embodiments, the hydrogel has a biodegradation half-life in the range of from about 8 weeks to about 15 weeks when disposed within the vitreous cavity of an eye. In certain embodiments, the hydrogel has a biodegradation half-life of less than 1, 2, 3, 4, 5, 6, 7, 8, 9, 10, 11, 12, 13, 14, 15, 16, 17, 18, 19, 20, 21, 22, 23, or 24 weeks when disposed within the vitreous cavity of an eye. In certain embodiments, the hydrogel has a biodegradation half-life of less than 1, 2, 3, 4, 5, 6, 7, 8, 9, 10, 11, 12, 13, 14, 15, 16, 17, 18, 19, 20, 21, 22, 23, or 24 months when disposed within the vitreous cavity of an eye.

In yet other embodiments, the hydrogel turns into liquid after approximately 5 weeks at a temperature in the range of 20° C. to 25° C., or within from about 4 weeks to 10 weeks, including all values and ranges therein. In embodiments, the ester bonds remaining in the hydrogel may degrade at room temperature in solution, such as in a phosphate buffered saline solution. In embodiments, degradation may begin after a few days and the hydrogel may be almost fully degraded, that is they form soluble products and the hydrogel turns in to liquid at around five weeks at a temperature in the range of 20° C. to 25° C. The rate of degradation will depend on a number of parameters, including total crosslink density, number of ester linkages in the crosslinks and the specifics of the environment.

Deliberate inclusion of degradable constituents into the nucle-functional polymer, electro-functional polymer, and/or biocompatible polymer permits tuning of the degradability and longevity of these materials and/or hydrogel in their chosen application. Examples of degradable constituents can be in the crosslinks, or elsewhere and can include, for example, any molecule or group that contains an ester bond (e.g. carbamate, amide, carbonate, lactic acid, glycolic acid, caprolactone or others). In certain embodiments, the degradable elements may be incorporated at an amount in the range of 1 to 6 per crosslinker. Similarly, incorporation of other functional groups into the hydrogel, such as though modification of the poly(vinyl alcohol) or poly(ethylene glycol) provide further degrees of tuning of the properties of the hydrogel.

### Pressure Generated Within the Eye

The therapeutic methods and compositions can be characterized according to the amount of pressured generated by the hydrogel in eye of the subject. For example, in certain embodiments, the hydrogel generates a pressure within the eye of less than 25 mmHg. In some embodiments, the hydrogel generates a pressure within the eye of less than 35 mmHg. In certain other embodiments, the hydrogel generates a pressure within the eye in the range of from about 10 mmHg to about 25 mmHg. In some embodiments, the hydrogel generates a pressure within the eye in the range of from about 20 mmHg to about 35 mmHg. In certain other embodiments, the hydrogel generates a pressure within the eye of about 15, 16, 17, 18, 29, 20, 21, 22, 23, 24, or 25, 26, 27, 28, 29, 30, 31, 32, 33, 34, or 35 mmHg. Methods and devices for measuring intraocular pressure are known in the art and include a tonometer such as a Tono-Pen.

It is contemplated that upon initial formation of the hydrogel in the eye of a subject, the hydrogel will be in a hyperosmotic state, where the concentration of hydrogel is such that additional fluid is pulled in (if available) by the gel to swell it. This approach allows the injected hydrogel to be filled passively to the size of the cavity, and then pull in additional water to exert an active swelling pressure on the interior of the eye suitable for the tamponade affect. In certain embodiments, the amount of swelling of the hydrogel is >5% and <20% within the first 24 hours of initial formation. The extent of the hyperosmotic state would be tunable using the concentration of the active ingredients. The source of the water in vivo would be the natural aqueous production in the eye, which is known to be produced at a rate of approximately 2-3 μL/min

### Features of the Nucleo-Functional Polymer

The therapeutic methods, compositions, and formulations for forming a hydrogel can be characterized according to features of the nucleo-functional polymer. Accordingly, in certain embodiments, the nucleo-functional polymer is a biocompatible poly(vinyl alcohol) polymer substituted by a plurality of thio-functional groups —R¹—SH. In certain embodiments, the nucleo-functional polymer is a biocompatible, partially hydrolyzed poly(vinyl alcohol) polymer substituted by a plurality of thio-functional groups —R¹—SH. In certain embodiments, the nucleo-functional polymer is a biocompatible, partially hydrolyzed poly(vinyl alcohol) polymer substituted by a plurality of thio-functional groups —R¹—SH, wherein the degree of hydrolysis of the partially hydrolyzed poly(vinyl alcohol) polymer is at least 85%, 88%, 90%, 92%, 95%, 97%, 98%, or 99%. In certain embodiments, the nucleo-functional polymer is a biocompatible, partially hydrolyzed poly(vinyl alcohol) polymer substituted by a plurality of thio-functional groups —R¹—SH, wherein the degree of hydrolysis of the partially hydrolyzed poly(vinyl alcohol) polymer is at least 85%. In certain embodiments, the nucleo-functional polymer is a biocompatible, partially hydrolyzed poly(vinyl alcohol) polymer substituted by a plurality of thio-functional groups —R¹—SH, wherein the degree of hydrolysis of the partially hydrolyzed poly(vinyl alcohol) polymer is at least 90%. In certain embodiments, the nucleo-functional polymer is a biocompatible, partially hydrolyzed poly(vinyl alcohol) polymer substituted by a plurality of thio-functional groups —R¹—SH, wherein the degree of hydrolysis of the partially hydrolyzed poly(vinyl alcohol) polymer is at least 95%. In certain embodiments, the nucleo-functional polymer is a biocompatible, partially hydrolyzed poly(vinyl alcohol) polymer substituted by a plurality of thio-functional groups —R¹—SH, wherein the degree of hydrolysis of the partially hydrolyzed poly(vinyl alcohol) polymer is at least 98%. In certain embodiments, the nucleo-functional polymer is a biocompatible, partially hydrolyzed poly(vinyl alcohol) polymer substituted by a plurality of thio-functional groups —R¹—SH, wherein the degree of hydrolysis of the partially hydrolyzed poly(vinyl alcohol) polymer is at least 99%.

In certain embodiments, the nucleo-functional polymer is a biocompatible polyalkylene polymer substituted by (i) a plurality of —OH groups, (ii) a plurality of thio-functional groups —R¹—SH, (iii) at least one polyethylene glycolyl group, and (iv) one or more —OC(O)—(C₁-C₆ alkyl) groups. In some embodiments, the nucleo-functional polymer is a biocompatible poly(vinyl alcohol) polymer substituted by (i) a plurality of thio-functional groups —R¹—SH and (ii) at least one polyethylene glycolyl group.

In certain other embodiments, the nucleo-functional polymer is a biocompatible, partially hydrolyzed poly(vinyl alcohol) polymer substituted by (i) a plurality of thio-functional groups —R¹—SH and (ii) at least one polyethylene glycolyl group. In certain embodiments, the degree of hydrolysis of the partially hydrolyzed poly(vinyl alcohol) polymer is at least 80%. In certain embodiments, the degree of hydrolysis of the partially hydrolyzed poly(vinyl alcohol) polymer is at least 85%. In certain embodiments, the degree of hydrolysis of the partially hydrolyzed poly(vinyl alcohol) polymer is at least 90%. In certain embodiments, the degree of hydrolysis of the partially hydrolyzed poly(vinyl alcohol) polymer is at least 95%. In certain embodiments, the degree of hydrolysis of the partially hydrolyzed poly(vinyl alcohol) polymer is at least 98%. In certain embodiments, the degree of hydrolysis of the partially hydrolyzed poly(vinyl alcohol) polymer is in the range of about 85% to about 91%.

The nucleo-functional polymer may be further characterized according to the number of polyethylene glycolyl groups in the nucleo-functional polymer. Accordingly, in certain embodiments, the nucleo-functional polymer contains from one to ten polyethylene glycolyl groups. In certain embodiments, the nucleo-functional polymer contains from one to five polyethylene glycolyl groups. In certain embodiments, the nucleo-functional polymer contains from one polyethylene glycolyl group.

In certain embodiments, the thio-functional group —R¹—SH is —OC(O)—(C₁-C₆ alkylene)-SH. In certain embodiments, the thio-functional group —R¹—SH is —OC(O)—(CH₂CH₂)—SH.

As described in the literature, poly(vinyl alcohol) is prepared by first polymerizing vinyl acetate to produce poly(vinyl acetate), and then the poly(vinyl acetate) is subjected to hydrolytic conditions to cleave the ester bond of the acetate group leaving only a hydroxyl group bound to the polymer backbone. Depending on the hydrolytic conditions used to cleave the ester bond of the acetate group, the resulting polymer product may still contain some acetate groups. That is, not all the acetate groups on the polymer are cleaved. For this reason, per common nomenclature used in the literature, the poly(vinyl alcohol) can be further characterized according to whether it is (a) fully hydrolyzed (i.e., all the acetate groups from the starting poly(vinyl acetate) starting material that have been converted to hydroxyl groups)) or (b) partially hydrolyzed (i.e., where some percentage of acetate groups from the poly(vinyl acetate) starting material have not been converted to hydroxyl groups). A partially hydrolyzed poly(vinyl alcohol) can be referred to as a poly(vinyl alcohol-co-vinyl acetate)). Per common usage in the literature, a poly(vinyl alcohol) that is partially hydrolyzed can be characterized according to the degree of hydrolysis (i.e., the percentage of acetate groups from the starting poly(vinyl acetate) starting material that have been converted to hydroxyl groups), such as greater than about 70%, 75%, 80%, 81%, 82%, 83%, 84%, 85%, 86%, 87%, 88%, 89%, 90%, 91%, 92%, 93%, 94%, 95%, 96%, 97%, 98%, or 99%. In certain embodiments, the degree of hydrolysis is in the range of from about 75% to about 95%, about 80% to about 95%, about 80% to about 90%, about 80% to about 85%, about 85% to about 95%, or about 85% to about 90%. For clarity, the term “poly(vinyl alcohol)” used herein encompasses both (a) fully hydrolyzed (i.e., all the acetate groups from the starting poly(vinyl acetate) starting material have been converted to hydroxyl groups)) and (b) partially hydrolyzed (i.e., where some percentage of acetate groups from the poly(vinyl acetate) starting material have not been converted to hydroxyl groups) material, unless indicated otherwise.

In certain embodiments, the nucleo-functional polymer is a biocompatible poly(vinyl alcohol) polymer comprising:

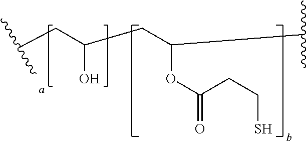

wherein a is an integer from 1-20 and b is an integer from 1-20. In certain embodiments, the nucleo-functional polymer is a biocompatible poly(vinyl alcohol) polymer comprising (i) a polyethylene glycolyl substituent and (ii)

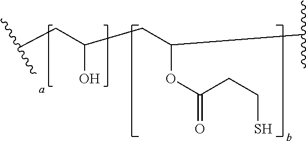

wherein a is an integer from 1-20 and b is an integer from 1-20.

In certain embodiments, the nucleo-functional polymer is a biocompatible poly(vinyl alcohol) polymer comprising:

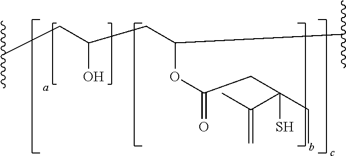

wherein a is an integer from 1-20, b is an integer from 1-20, and c is an integer from about 20 to about 500. In certain embodiments, the nucleo-functional polymer is a biocompatible poly(vinyl alcohol) polymer comprising (i) a polyethylene glycolyl substituent and (ii)

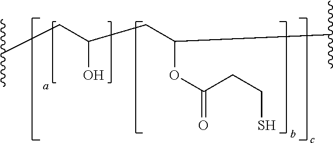

wherein a is an integer from 1-20, b is an integer from 1-20, and c is an integer from about 20 to about 500.

In certain embodiments, the nucleo-functional polymer is a biocompatible poly(vinyl alcohol) polymer comprising:

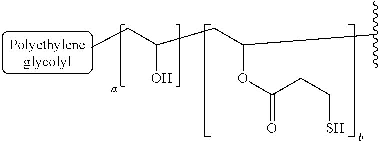

wherein a is an integer from 1-20 and b is an integer from 1-20.

In certain embodiments, the nucleo-functional polymer is a biocompatible poly(vinyl alcohol) polymer comprising:

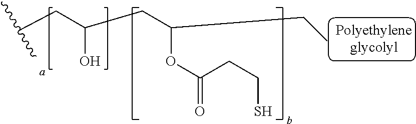

wherein a is an integer from 1-20 and b is an integer from 1-20.

In certain embodiments, the nucleo-functional polymer is a biocompatible poly(vinyl alcohol) polymer comprising:

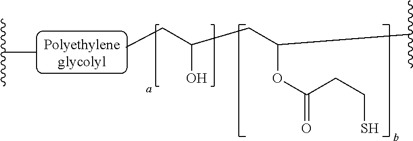

wherein a is an integer from 1-20 and b is an integer from 1-20.

In certain embodiments, the nucleo-functional polymer is a biocompatible poly(vinyl alcohol) polymer comprising:

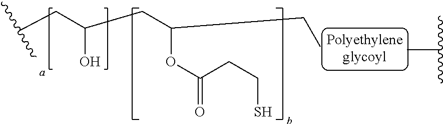

wherein a is an integer from 1-20 and b is an integer from 1-20.

The nucleo-functional polymer may be further characterized according to its molecular weight, such as the weight-average molecular weight of the polymer. In certain embodiments, the nucleo-functional polymer has a weight-average molecular weight in the range of from about 500 g/mol to about 1,000,000 g/mol. In certain embodiments, the nucleo-functional polymer has a weight-average molecular weight in the range of from about 2,000 g/mol to about 500,000 g/mol. In certain embodiments, the nucleo-functional polymer has a weight-average molecular weight in the range of from about 4,000 g/mol to about 30,000 g/mol. In certain embodiments, the nucleo-functional polymer has a weight-average molecular weight less than about 200,000 g/mol or less than about 100,000 g/mol. In certain embodiments, the nucleo-functional polymer has a weight-average molecular weight in the range of from about 20,000 g/mol to about 75,000 g/mol. In certain embodiments, the nucleo-functional polymer has a weight-average molecular weight in the range of from about 25,000 g/mol to about 55,000 g/mol. In certain embodiments, the nucleo-functional polymer has a weight-average molecular weight in the range of from about 25,000 g/mol to about 35,000 g/mol. In certain embodiments, the nucleo-functional polymer has a weight-average molecular weight in the range of from about 29,000 g/mol to about 33,000 g/mol. In certain embodiments, the nucleo-functional polymer has a weight-average molecular weight of about 31,000 g/mol. In certain embodiments, the nucleo-functional polymer has a weight-average molecular weight in the range of from about 26,000 g/mol to about 32,000 g/mol. In certain embodiments, the nucleo-functional polymer has a weight-average molecular weight of about 29,000 g/mol. In certain embodiments, the nucleo-functional polymer has a weight-average molecular weight of about 30,000 g/mol. In certain embodiments, the nucleo-functional polymer has a weight-average molecular weight in the range of from about 45,000 g/mol to about 55,000 g/mol. In certain embodiments, the nucleo-functional polymer has a weight-average molecular weight of about 50,000 g/mol.

The nucleo-functional polymer may be further characterized according to the molecular weight of any polyethylene glycolyl group. For example, in certain embodiments, the polyethylene glycolyl group has a weight-average molecular weight in the range of from about 100 g/mol to about 10,000 g/mol. In certain embodiments, the polyethylene glycolyl group has a weight-average molecular weight in the range of from about 100 g/mol to about 1,000 g/mol. In certain embodiments, the polyethylene glycolyl group has a weight-average molecular weight in the range of from about 1,000 g/mol to about 2,000 g/mol. In certain embodiments, the polyethylene glycolyl group has a weight-average molecular weight in the range of from about 2,000 g/mol to about 3,000 g/mol. In certain embodiments, the polyethylene glycolyl group has a weight-average molecular weight in the range of from about 3,000 g/mol to about 4,000 g/mol. In certain embodiments, the polyethylene glycolyl group has a weight-average molecular weight in the range of from about 4,000 g/mol to about 5,000 g/mol. In certain embodiments, the polyethylene glycolyl group has a weight-average molecular weight in the range of from about 5,000 g/mol to about 6,000 g/mol. In certain embodiments, the polyethylene glycolyl group has a weight-average molecular weight in the range of from about 6,000 g/mol to about 7,000 g/mol. In certain embodiments, the polyethylene glycolyl group has a weight-average molecular weight in the range of from about 7,000 g/mol to about 8,000 g/mol. In certain embodiments, the polyethylene glycolyl group has a weight-average molecular weight in the range of from about 8,000 g/mol to about 9,000 g/mol. In certain embodiments, the polyethylene glycolyl group has a weight-average molecular weight in the range of from about 9,000 g/mol to about 10,000 g/mol. In certain embodiments, the polyethylene glycolyl group has a weight-average molecular weight in the range of from about 5,000 g/mol to about 7,000 g/mol.

In certain embodiments, the nucleo-functional polymer is a thiolated poly(vinyl alcohol) that has been fully hydrolyzed or partially hydrolyzed (e.g., hydrolysis of about 75% or more, including all values and ranges from 75% to 99.9%, including 75%, 80%, 85%, 90%, 95%, 96%, 97%, 98%, 99%, etc.). In certain embodiments, the poly(vinyl alcohol) polymer is substantially fully hydrolyzed having, for example, less than 1.5 acetate groups remaining. The thiolated poly(vinyl alcohol) may be further characterized according to its molecular weight, such as where the thiolated poly(vinyl alcohol) has a weight average molecular weight (Mw) the range of 2 kDa to 2,000,000 kDa, including all values and ranges therein, and such as 2 kDa to 1,000,000 kDa, 2 kDa to 200 kDa, and 30 kDa to 50 kDa, etc. The thiolated poly(vinyl alcohol) may be further characterized according to its thiolation percentage. In certain embodiments, the thiolated poly(vinyl alcohol) has a thiolation percentage of up to about 30%. In some embodiments, the thiolated poly(vinyl alcohol) has a thiolation percentage of about 1% to about 30%. In certain embodiments, the thiolated poly(vinyl alcohol) has a thiolation percentage of about 1% to about 25%, about 1% to about 20%, about 1% to about 15%, about 1% to about 10%, or about 1% to about 5%. In some embodiments, the thiolated poly(vinyl alcohol) has a thiolation percentage of about 5% to about 10% or about 5% to about 7%.

The thiolated poly(vinyl alcohol) can be prepared by reacting a range of thiol containing functional groups with poly(vinyl alcohol), as further described in U.S. Patent Application Publication No. 2016/0009872, which is hereby incorporated by reference. In certain embodiments, thiolated poly(vinyl alcohol) is prepared by reacting (a) a compound having a thiol functionality and at least one hydroxyl-reactive group, such as, for example, a carboxyl group, represented by HS—R—CO₂H, where R may include an alkane, unsaturated ether, or ester group, and R includes from 1 to 20 carbons, with (b) a poly(vinyl alcohol).

In certain embodiments, the thiolated poly(vinyl alcohol) comprises the following fragment:

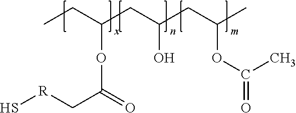

wherein R includes 1 to 20 carbons and may be an alkane, saturated ether or ester, and the individual units are randomly distributed along the length of the poly(vinyl alcohol) chain. X is in the range of 0.1-10%, n is in the range of 80-99.9%, indicating the level of hydrolysis of the poly(vinyl alcohol) polymer and allowing for water solubility of the polymer and m, the amount of non-hydrolyzed acetate groups, is in the range 0.1-20%.

The amount of thiol groups on the poly(vinyl alcohol) can be controlled by the number of hydroxyl groups on the poly(vinyl alcohol) that undergo reaction with the thiolating agent to generate the thiolated poly(vinyl alcohol). In certain embodiments, the amount of thiol functional groups on the poly(vinyl alcohol) may be characterized according to the molar ratio of thiol functional groups to poly(vinyl alcohol) polymer, such as from about 0.1:1 to about 10.0:1, including all values and ranges therein. Furthermore, the amount of thiol groups on the poly(vinyl alcohol) can be regulated by the reaction temperature and reaction time used when reacting the thiolating agent with the poly(vinyl alcohol) to form the thiolated poly(vinyl alcohol). In certain embodiments, the reaction 0 temperature may be in the range of 40° C. to 95° C., and reaction time may be in the range of 5 hours to 48 hours, including all values and ranges therein. Of course, cooler reaction temperatures may be utilized as well, such as in the range of 20° C. up to 40° C.

In certain embodiments, the nucleo-functional polymer is polyvinyl alcohol-polyethylene glycol graft-copolymer substituted by (i) a plurality of thio-functional groups —R¹—SH, wherein R¹ is an ester-containing linker. In certain embodiments, the thio-functional group-R¹—SH is —OC(O)—(CH₂CH₂)—SH. In certain embodiments, the polyethylene glycol has a weight-average molecular weight in the range of about 4,000 g/mol to about 8,000 g/mol. In certain embodiments, the polyethylene glycol has a weight-average molecular weight in the range of about 5,000 g/mol to about 7,000 g/mol. In certain embodiments, the polyethylene glycol has a weight-average molecular weight of about 6,000 g/mol.

In certain embodiments, the nucleo-functional polymer has a weight-average molecular weight in the range of from about 500 g/mol to about 1,000,000 g/mol. In certain embodiments, the nucleo-functional polymer has a weight-average molecular weight in the range of from about 2,000 g/mol to about 500,000 g/mol. In certain embodiments, the nucleo-functional polymer has a weight-average molecular weight in the range of from about 25,000 g/mol to about 75,000 g/mol. In certain embodiments, the nucleo-functional polymer has a weight-average molecular weight in the range of from about 40,000 g/mol to about 60,000 g/mol. In certain embodiments, the nucleo-functional polymer has a weight-average molecular weight in the range of from about 40,000 g/mol to about 50,000 g/mol. In certain embodiments, the nucleo-functional polymer has a weight-average molecular weight of about 45,000 g/mol. In certain embodiments, the nucleo-functional polymer has a weight-average molecular weight in the range of from about 45,000 g/mol to about 55,000 g/mol. In certain embodiments, the nucleo-functional polymer has a weight-average molecular weight of about 50,000 g/mol.

In certain other embodiments, the nucleo-functional polymer has a weight-average molecular weight in the range of from about 4,000 g/mol to about 30,000 g/mol. In certain embodiments, the nucleo-functional polymer has a weight-average molecular weight less than about 200,000 g/mol or less than about 100,000 g/mol. In certain embodiments, the nucleo-functional polymer has a weight-average molecular weight in the range of from about 26,000 g/mol to about 32,000 g/mol. In certain embodiments, the nucleo-functional polymer has a weight-average molecular weight of about 29,000 g/mol. In certain embodiments, the nucleo-functional polymer has a weight-average molecular weight of about 30,000 g/mol.

In certain embodiments, the number of hydroxyl groups on the nucleo-functional polymer is in the range of two-fold to eight-fold greater than the number of thio-functional groups —R¹—SH on the nucleo-functional polymer. In certain embodiments, the number of hydroxyl groups on the nucleo-functional polymer is in the range of three-fold to five-fold greater than the number of thio-functional groups —R¹—SH on the nucleo-functional polymer. In certain embodiments, the number of hydroxyl groups on the nucleo-functional polymer is about three-fold greater than the number of thio-functional groups —R¹—SH on the nucleo-functional polymer. In certain embodiments, the number of hydroxyl groups on the nucleo-functional polymer is about four-fold greater than the number of thio-functional groups —R¹—SH on the nucleo-functional polymer.

In some embodiments, the nucleo-functional polymer is a polyethylene glycol substituted polyvinyl alcohol having a saponification degree of 86.5 to 89.5 mole percent and a weight-average molecular weight of about 50,000 g/mol sold by Gohsenol under product number WO-320R, in which a plurality of the hydroxyl groups have been converted to —OC(O)CH₂CH₂SH groups. In certain embodiments, the nucleo-functional polymer is polyethylene glycol substituted polyvinyl alcohol having a saponification degree of at least 98.5 mole percent and a weight-average molecular weight of about 50,000 g/mol sold by Gohsenol under product number WO-320N, in which a plurality of the hydroxyl groups have been converted to —OC(O)CH₂CH₂SH groups.

In some embodiments, the nucleo-functional polymer is a polyvinyl alcohol-polyethylene glycol graft-copolymer having a weight-average molecular weight of about 45,000 g/mol sold by BASF under the tradename KOLLICOAT® IR, in which a plurality of the hydroxyl groups have been converted to —OC(O)CH₂CH₂SH groups.

In certain embodiments, the nucleo-functional polymer containing a plurality of thio-functional groups can be prepared based on procedures described in the literature, such as U.S. Patent Application 2016/0009872 in which a polymer having nucleophilic groups (e.g., hydroxyl groups) is reacted with a thiol-containing compound so that resulting polymer contains a thiol group bound to the polymer backbone via a linker.

### Features of the Electro-Functional Polymer

The therapeutic methods and compositions for forming a hydrogel can be characterized according to features of the electro-functional polymer. Accordingly, in certain embodiments, the electro-functional polymer is a biocompatible polymer selected from a polyalkylene and polyheteroalkylene polymer each being substituted by at least one thiol-reactive group. In certain embodiments, the electro-functional polymer is a biocompatible polyheteroalkylene polymer substituted by at least one thiol-reactive group. In certain embodiments, the electro-functional polymer is a biocompatible poly(oxyalkylene) polymer substituted by at least one thiol-reactive group. In certain embodiments, the electro-functional polymer is a biocompatible poly(ethylene glycol) polymer substituted by at least one thiol-reactive group.

In certain embodiments, the thiol-reactive group is an alpha-beta unsaturated ester, maleimidyl, or,

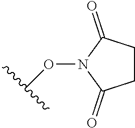 [SMILES: *ON1C(=O)CCC1=O]

each of which is optionally substituted by one or more occurrences of alkyl, aryl, or aralkyl. In certain embodiments, the thiol-reactive group is an alpha-beta unsaturated ester optionally substituted by one or more occurrences of alkyl, aryl, or aralkyl. In certain embodiments, the thiol-reactive group is —OC(O)CH═CH₂.

In certain embodiments, the electro-functional polymer has the formula:

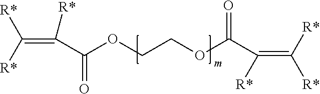

wherein R* is independently for each occurrence hydrogen, alkyl, aryl, or aralkyl; and m is an integer in the range of 5 to 15,000. In certain embodiments, R* is hydrogen. In yet other embodiments, m is an integer in the range of from about 20 to about 100, about 100 to about 500, about 500 to about 750, about 750 to about 1,000, about 1,000 to about 2,000, about 2,000 to about 5,000, about 5,000 to about 7,500, about 7,500 to about 10,000, about 10,000 to about 12,500, about 12,500 to about 15,000.

The electro-functional polymer may be further characterized according to its molecular weight, such the weight-average molecular weight of the polymer. Accordingly, in certain embodiments, the electro-functional polymer has a weight-average molecular weight in the range of from about 500 g/mol to about 1,000,000 g/mol. In certain embodiments, the electro-functional polymer has a weight-average molecular weight in the range of from about 1,000 g/mol to about 100,000 g/mol. In certain embodiments, the electro-functional polymer has a weight-average molecular weight in the range of from about 2,000 g/mol to about 8,000 g/mol. In certain embodiments, the electro-functional polymer has a weight-average molecular weight less than about 200,000 g/mol or less than about 100,000 g/mol. In certain embodiments, the electro-functional polymer has a weight-average molecular weight in the range of from about 1,000 g/mol to about 15,000 g/mol. In certain embodiments, the electro-functional polymer has a weight-average molecular weight in the range of from about 2,000 g/mol to about 8,000 g/mol. In certain embodiments, the electro-functional polymer has a weight-average molecular weight in the range of from about 3,000 g/mol to about 4,000 g/mol. In certain embodiments, the electro-functional polymer has a weight-average molecular weight in the range of from about 3,200 g/mol to about 3,800 g/mol. In certain embodiments, the electro-functional polymer has a weight-average molecular weight of about 3,400 g/mol. In some embodiments, the electro-functional polymer has a weight-average molecular weight of about 3,500 g/mol.

The electro-functional polymer may be a straight-chain polymer or a branched chain polymer. In yet other embodiments, the electro-functional polymer may be a multi-arm polymer described in U.S. Pat. No. 9,072,809, which is hereby incorporated by reference, such as pentaerythritol poly(ethylene glycol) maleimide (4ARM-PEG-MAL) (molecular weight selected from about 5,000 to about 40,000, e.g., 10,000 or 20,000), pentaerythritol poly(ethylene glycol) succinimidyl succinate (4ARM-PEG-SS) (molecular weight selected from about 5,000 to about 40,000, e.g., 10,000 or 20,000), pentaerythritol poly(ethylene glycol) succinimidyl glutarate (4ARM-PEG-SG) (molecular weight selected from about 5,000 to about 40,000, e.g., 10,000 or 20,000), pentaerythritol poly(ethylene glycol) succinimidyl glutaramide (4ARM-PEG-SGA) (molecular weight selected from about 5,000 to about 40,000, e.g., 10,000 or 20,000), hexaglycerin poly(ethylene glycol) succinimidyl succinate (8ARM-PEG-SS) (molecular weight selected from about 5,000 to about 40,000, e.g., 10,000 or 20,000), hexaglycerin poly(ethylene glycol) succinimidyl glutarate (8ARM-PEG-SG) (molecular weight selected from about 5,000 to about 40,000, e.g., 10,000, 15,000, 20,000, or 40,000), hexaglycerin poly(ethylene glycol) succinimidyl glutaramide (8ARM-PEG-SGA) (molecular weight selected from about 5,000 to about 40,000, e.g., 10,000, 15,000, 20,000, or 40,000), tripentaerythritol poly(ethylene glycol) succinimidyl succinate (8ARM(TP)-PEG-SS) (molecular weight selected from about 5,000 to about 40,000, e.g., 10,000 or 20,000), tripentaerythritol poly(ethylene glycol) succinimidyl glutarate (8ARM(TP)-PEG-SG) (molecular weight selected from about 5,000 to about 40,000, e.g., 10,000, 15,000, 20,000, or 40,000), or tripentaerythritol poly(ethylene glycol) succinimidyl glutaramide (8ARM(TP)-PEG-SGA) (molecular weight selected from about 5,000 to about 40,000, e.g., 10,000, 15,000, 20,000, or 40,000).

In other embodiments, the electro-functional polymer may be a poly(ethylene glycol) end-capped with at least two thiol-reactive groups. The poly(ethylene glycol) may be linear, branched, a dendrimer, or multi-armed. The thiol reactive group may be, for example, an acrylate, methacrylate, maleimidyl, haloacetyl, pyridyldithiol, or N-hydroxysuccinimidyl. An exemplary poly(ethylene glycol) end-capped with thiol-reactive groups may be represented by the formula Y—[—O—CH₂CH₂—]ₙ—O—Y wherein each Y is a thiol-reactive group, and n is, for example, in the range of 200 to 20,000. In another more specific embodiment, the electro-functional polymer may be CH₂═CHC(O)O—[—CH₂CH₂—O—]_{b}—C(O)CH═CH₂, wherein b is, for example, in the range of about 200 to about 20,000. Alternatively or additionally to the linear embodiments depicted above, the poly(ethylene glycol) may be a dendrimer. For example, the poly(ethylene glycol) may be a 4 to 32 hydroxyl dendron. In further embodiments, the poly(ethylene glycol) may be multi-armed. In such embodiments, the poly(ethylene glycol) may be, for example, a 4, 6 or 8 arm and hydroxy-terminated. The molecular weight of the poly(ethylene glycol) may be varied, and in some cases one of the thiol-reactive groups may be replaced with other structures to form dangling chains, rather than crosslinks. In certain embodiments, the molecular weight (Mw) is less than 20,000, including all values and ranges from 200 to 20,000, such as 200 to 1,000, 1,000 to 10,000, etc. In addition, the degree of functionality may be varied, meaning that the poly(ethylene glycol) may be mono-functional, di-functional or multi-functional.

In certain embodiments, the electro-functional polymer can be purchased from commercial sources or prepared based on procedures described in the literature, such as by treating a nucleo-functional polymer with reagent(s) to install one or more electrophilic groups (e.g., by reacting poly(ethylene glycol) with acrylic acid in an esterification reaction to form poly(ethylene glycol) diacrylate).

### Relative Amount of Nucleo-Functional Polymer and Electro-Functional Polymer

The therapeutic methods and compositions for forming a hydrogel can be characterized according to relative amount of nucleo-functional polymer and electro-functional polymer used. Accordingly, in certain embodiments, the mole ratio of (i) thio-functional groups —R¹—SH to (ii) thiol-reactive group is in the range of 10:1 to 1:10. In certain embodiments, the mole ratio of (i) thio-functional groups —R¹—SH to (ii) thiol-reactive groups is in the range of 5:1 to 1:1. In certain embodiments, the mole ratio of (i) thio-functional groups —R¹—SH to (ii) thiol-reactive groups is in the range of 2:1 to 1:1.

In certain embodiments, a thiolated poly (vinyl alcohol) and poly(ethylene glycol)-diacrylate are delivered at a ratio of functional groups (mmol/mmol) in the range of 2:1 to 0.5:1, including all values and ranges therein, and preferably 1:1. In some embodiments, a 6% thiolated poly (vinyl alcohol) with a range of about 5%-7% thiol modification (thiolation percentage) and a 6% poly(ethylene glycol)-diacrylate are provided and/or used in combination. Furthermore, once combined the combination of the thiolated poly(vinyl alcohol) and the poly(ethylene glycol)-diacrylate are present in solution in the range of about 6 mg/mL to about 250 mg/mL, including all values and ranges therein, and preferably about 25 mg/mL to about 65 mg/mL, and sometimes about 45 mg/mL. The viscosity of the thiolated poly(vinyl alcohol) and the poly(ethylene glycol)-diacrylate, prior to crosslinking and gelation, is in the range of about 0.005 Pa*s to about 0.35 Pa*s, including all values and ranges therein, such as in the range of about 0.010 Pa*s to about 0.040 Pa*s, and sometimes about 0.028 Pa*s.

### Amount of Nucleo-functional Polymer in the Ocular Formulation or Pharmaceutical Composition

The therapeutic methods and compositions for forming a hydrogel can be characterized according to amount of nucleo-functional polymer in the ocular formulation. Accordingly, in certain embodiments, the ocular formulation comprises the nucleo-functional polymer in an amount of from about 0.5% w/v to about 15% w/v. In certain embodiments, the ocular formulation comprises the nucleo-functional polymer in an amount of from about 1% w/v to about 10% w/v. In certain embodiments, the ocular formulation comprises the nucleo-functional polymer in an amount of from about 1% w/v to about 3% w/v. In certain embodiments, the ocular formulation comprises the nucleo-functional polymer in an amount of from about 3% w/v to about 5% w/v. In certain embodiments, the ocular formulation comprises the nucleo-functional polymer in an amount of from about 5% w/v to about 7% w/v. In certain embodiments, the ocular formulation comprises the nucleo-functional polymer in an amount of from about 7% w/v to about 9% w/v. In certain embodiments, the ocular formulation comprises the nucleo-functional polymer in an amount of from about 9% w/v to about 11% w/v.

### Amount of Electro-functional Polymer in the Ocular Formulation or Pharmaceutical Composition

The therapeutic methods and compositions for forming a hydrogel can be characterized according to presence and/or amount of electro-functional polymer in the ocular formulation. Accordingly, in certain embodiments, the ocular formulation comprises the electro-functional polymer. In certain embodiments, the ocular formulation comprises the electro-functional polymer in an amount of from about 0.5% w/v to about 15% w/v. In certain embodiments, the ocular formulation comprises the electro-functional polymer in an amount of from about 1% w/v to about 10% w/v. In certain embodiments, the ocular formulation comprises the electro-functional polymer in an amount of from about 1% w/v to about 3% w/v. In certain embodiments, the ocular formulation comprises the electro-functional polymer in an amount of from about 3% w/v to about 5% w/v. In certain embodiments, the ocular formulation comprises the electro-functional polymer in an amount of from about 5% w/v to about 7% w/v. In certain embodiments, the ocular formulation comprises the electro-functional polymer in an amount of from about 7% w/v to about 9% w/v.

### Administration Features of Nucleo-functional Polymer and Electro-functional Polymer

The method may be further characterized according to whether the nucleo-functional polymer and the electro-functional polymer are administered together as a single composition to the vitreous cavity of the eye of the subject, or alternatively the nucleo-functional polymer and the electro-functional polymer are administered separately to the vitreous cavity of the eye of the subject. In certain embodiments, the nucleo-functional polymer and the electro-functional polymer are administered together as a single composition to the vitreous cavity of the eye of the subject. The single composition may further comprise, for example, a liquid pharmaceutically acceptable carrier for administration to the eye of a subject. In certain embodiments, the nucleo-functional polymer and the electro-functional polymer are administered together as a single, liquid aqueous pharmaceutical composition to the vitreous cavity of the eye of the subject.

In certain other embodiments, the nucleo-functional polymer and the electro-functional polymer are administered separately to the vitreous cavity of the eye of the subject. Even when administered separately, the electro-functional polymer may be administered as a liquid ocular formulation comprising a liquid pharmaceutically acceptable carrier for administration to the eye of a subject. This facilitates easy administration of the electro-functional polymer through surgical ports in the eye of the subject. Similarly, the electro-functional polymer may be administered as a liquid ocular formulation comprising a liquid pharmaceutically acceptable carrier for administration to the eye of a subject. This facilitates easy administration of the electro-functional polymer through surgical ports in the eye of the subject. Accordingly, in certain embodiments, the nucleo-functional polymer and the electro-functional polymer are administered separately to the vitreous cavity of the eye of the subject, wherein the nucleo-functional polymer is administered as a single, liquid aqueous pharmaceutical composition to the vitreous cavity of the eye of the subject, and the electro-functional polymer is administered as a single, liquid aqueous pharmaceutical composition to the vitreous cavity of the eye of the subject.

### Poly(Ethylene Glycol) Polymer

The methods and ocular formulation may be further characterized according to the identity and amount of poly(ethylene glycol) polymer. Accordingly, in certain embodiments, the ocular formulation comprises the poly(ethylene glycol) polymer in an amount of from about 0.5% w/v to about 30% w/v. In certain embodiments, the ocular formulation comprises the poly(ethylene glycol) polymer in an amount of from about 0.5% w/v to about 1% w/v. In certain embodiments, the ocular formulation comprises the poly(ethylene glycol) polymer in an amount of from about 1% w/v to about 3% w/v. In certain embodiments, the ocular formulation comprises the poly(ethylene glycol) polymer in an amount of from about 3% w/v to about 5% w/v. In certain embodiments, the ocular formulation comprises the poly(ethylene glycol) polymer in an amount of from about 5% w/v to about 7% w/v. In certain embodiments, the ocular formulation comprises the poly(ethylene glycol) polymer in an amount of from about 7% w/v to about 9% w/v. In certain embodiments, the ocular formulation comprises the poly(ethylene glycol) polymer in an amount of from about 10% w/v to about 15% w/v. In certain embodiments, the ocular formulation comprises the poly(ethylene glycol) polymer in an amount of from about 15% w/v to about 20% w/v. In certain embodiments, the ocular formulation comprises the poly(ethylene glycol) polymer in an amount of from about 20% w/v to about 25% w/v. In certain embodiments, the ocular formulation comprises the poly(ethylene glycol) polymer in an amount of from about 25% w/v to about 30% w/v.

In certain embodiments, the poly(ethylene glycol) polymer has a number-average molecular weight in the range of from about 200 g/mol to about 1,000 g/mol. In certain embodiments, the poly(ethylene glycol) polymer has a number-average molecular weight in the range of from about 200 g/mol to about 300 g/mol. In certain embodiments, the poly(ethylene glycol) polymer has a number-average molecular weight in the range of from about 300 g/mol to about 400 g/mol. In certain embodiments, the poly(ethylene glycol) polymer has a number-average molecular weight in the range of from about 400 g/mol to about 500 g/mol. In certain embodiments, the poly(ethylene glycol) polymer has a number-average molecular weight in the range of from about 500 g/mol to about 600 g/mol. In certain embodiments, the poly(ethylene glycol) polymer has a number-average molecular weight in the range of from about 600 g/mol to about 700 g/mol. In certain embodiments, the poly(ethylene glycol) polymer has a number-average molecular weight in the range of from about 700 g/mol to about 800 g/mol. In certain embodiments, the poly(ethylene glycol) polymer has a number-average molecular weight in the range of from about 800 g/mol to about 900 g/mol. In certain embodiments, the poly(ethylene glycol) polymer has a number-average molecular weight in the range of from about 900 g/mol to about 1,000 g/mol. In certain embodiments, the poly(ethylene glycol) polymer has a number-average molecular weight of about 400 g/mol.

### Features of the Ocular Formulation or Liquid Aqueous Pharmaceutical Composition

The ocular formulation or liquid aqueous pharmaceutical composition may be further characterized according to, for example, pH, osmolality and presence and/or identity of salts. In certain embodiments, the formulation has a pH in the range of about 7.1 to about 7.7. In certain embodiments, the formulation or liquid aqueous pharmaceutical composition has a pH in the range of about 7.3 to about 7.5. In certain embodiments, the formulation or liquid aqueous pharmaceutical composition has a pH of about 7.4. In certain embodiments, the formulation or liquid aqueous pharmaceutical composition further comprises an alkali metal salt. In certain embodiments, the formulation or liquid aqueous pharmaceutical composition further comprises an alkali metal halide salt, an alkaline earth metal halide salt, or a combination thereof. In certain embodiments, the formulation or liquid aqueous pharmaceutical composition further comprises sodium chloride. In certain embodiments, the formulation or liquid aqueous pharmaceutical composition further comprises sodium chloride, potassium chloride, calcium chloride, magnesium chloride, or a combination of two or more of the foregoing. In certain embodiments, the formulation or liquid aqueous pharmaceutical composition has an osmolality in the range of about 275 mOsm/kg to about 350 mOsm/kg. In certain embodiments, the formulation or liquid aqueous pharmaceutical composition has an osmolality in the range of about 275 mOsm/kg to about 315 mOsm/kg. In certain embodiments, the formulation or liquid aqueous pharmaceutical composition has an osmolality in the range of about 275 mOsm/kg to about 300 mOsm/kg. In certain embodiments, the formulation or liquid aqueous pharmaceutical composition has an osmolality in the range of about 275 mOsm/kg to about 295 mOsm/kg. In certain embodiments, the formulation or liquid aqueous pharmaceutical composition has an osmolality of about 290 mOsm/kg.

A liquid formulation or liquid aqueous pharmaceutical composition containing a nucleo-functional polymer and/or the electro-functional polymer may be further characterized according to the viscosity of the formulation. In certain embodiments, the liquid formulation has a viscosity within 10%, 25%, 50%, 75%, 100%, 150%, 200%, or 300% of water. In certain other embodiments, the liquid formulation has a viscosity such that it can be administered through a needle having a gauge of less than or equal to 23 using a force of no more than 5N. In some embodiments, the liquid formulation has a viscosity such that it can be administered through a needle having a gauge of less than or equal to 23 using a force of no more than 5 lbf or 22.5N. In certain embodiments, the liquid formulation has a viscosity such that 1-2 mL of the liquid formulation can be administered within 3 minutes using a needle having a gauge of less than or equal to 23 using a force of no more than 5N. In certain embodiments, the liquid formulation has a viscosity such that 1-2 mL of the liquid formulation can be administered within 3 minutes using a needle having a gauge of less than or equal to 23 using a force of no more than 5 lbf or 22.5N.

In certain embodiments, a nucleo-functional polymer and/or the electro-functional polymer are provided in an aqueous pharmaceutical composition for administration to the eye. Such aqueous pharmaceutical compositions are desirably low viscosity liquids. In embodiments, the liquids exhibit a viscosity in the range of 0.004 Pa*s to 0.5 Pa*s, including all values and ranges therein, such as 0.010 Pa*s to 0.05 Pa*s. For example, an aqueous pharmaceutical composition may desirably comprise poly(ethylene glycol) diacrylate at a concentration of 3 mg/mL to 300 mg/mL, including all values and ranges therein, such as in the range of 10 mg/mL to 50 mg/mL, and even the more specific value of about 30 mg/mL. Another more specific embodiment is a poly(ethylene glycol) diacrylate aqueous solution having a viscosity in the range of 0.007 Pa*s to 0.5 Pa*s, including all values and ranges therein, such as in the range of 0.01 Pa*s to 0.05 Pa*s, or the more specific value of about 0.035 Pa*s.

### Reducing the Amount of Dissolved Oxygen

It has been discovered that reducing the amount of dissolved oxygen in liquid materials used in the therapeutic methods can provide benefits, such as reducing degradation of the nucleo-functional polymer. Reducing the amount of dissolved oxygen can minimize formation of di-sulfide linkages/crosslinking of thiolated nucleo-functional polymers, for example, thiolated poly(vinyl alcohol). Accordingly, in certain embodiments, the aqueous pharmaceutically acceptable carrier (e.g., that used in the ocular formulation) has been treated to reduce the amount of dissolved oxygen. In certain embodiments, the aqueous pharmaceutically acceptable carrier has been sparged with an insert gas to reduce the amount of dissolved oxygen. In certain embodiments, the aqueous pharmaceutically acceptable carrier has been sparged with an argon gas to reduce the amount of dissolved oxygen.

In certain embodiments, any formulation for administration to a patient has been treated to reduce the amount of dissolved oxygen. In certain embodiments, such formulation has been sparged with an insert gas to reduce the amount of dissolved oxygen.

### Additional Features

It is appreciated that the properties and gelation times of the in situ formed gels can be regulated by the concentration of the nucleo-functional polymer, for example, thiolated poly(vinyl alcohol), and/or and poly(ethylene glycol)-diacrylate, their ratio used for cross-linking and functionality (amount of thiol groups linked to nucleo-functional polymer, for example, poly(vinyl alcohol), and the amount of thiol reactive groups per poly(ethylene glycol) molecule). By changing the nucleo-functional polymer (e.g., thiolated poly(vinyl alcohol)) to poly(ethylene glycol) ratio, one can also regulate the fraction of dangling poly(ethylene glycol) chains that can be used to improve hydrogel's surface properties. Furthermore, mixing a blend of mono-functional and bi-functional poly(ethylene glycol) crosslinkers, wherein the functionality is the thiol reactive groups will allow the tuning of the crosslinking versus hydrophilicity of the hydrogel. Control of the length of the mono-functional and bi-functional crosslinker or the size of the starting nucleo-functional polymer (e.g., poly(vinyl alcohol)), allows modification of mechanical properties, swelling, lubricity, morphology, and hydrophilicity as well as frictional and wear properties.

### Features of the Biocompatible Polymer

The therapeutic methods and compositions for forming a hydrogel can be characterized according to features of the biocompatible polymer. Exemplary biocompatible polymers for use in the therapeutic methods and compositions include:
xii. a thermosensitive polymer selected from a hydroxybutyl chitosan, carboxymethyl chitosan, chitosan-(D)-glucose phosphate, (chitosan)-(hydroxypropylmethyl cellulose)-(glycerin) polymer, chitosan-(beta-gly cerophosphate)-hydroxyethyl cellulose polymer, (hyaluronic acid)-(hyperbranched polyethylene glycol) copolymer, poloxamer, (poloxamer)-(chondroitan sulfate)-(polyethylene glycol) polymer, (poly(lactic acid))-(poloxamer)-(poly(lactic acid) polymer, (polyethylene glycol)-polyalanine copolymer, (polyethylene glycol)-(poly caprolactone)-(polyethylene glycol) polymer, (polyethylene glycol)-(polyester urethane) copolymer, [poly(beta-benzyl L-aspartate)]-(polyethylene glycol)-[poly(beta-benzyl L-aspartate)], polycaprolactone-(polyethylene glycol)-polycaprolactone polymer, poly(lactic-co-glycolic acid)-(polyethylene glycol)-(poly(lactic-co-glycolic acid)), polymethacrylamide-polmethacrylate copolymer, poly(methacrylamide-co-methacrylate)-gellan gum copolymer, thiolated gellan, acrylated poloxamine, poly(N-isopropylacrylamide), poly(phosphazene), collagen-(poly(glycolic acid)) copolymer, (glycosaminoglycan)-(polypeptide) polymer, (ulvan)-(polyisopropylacrylamide) copolymer, a mixture of poloxamers, a mixture of hyaluronic acid and (polycaprolactone-(polyethylene glycol)-polycaprolactone), and mixtures thereof;
xiii. a nucleo-functional polymer selected from a N—O carboxymethyl chitosan, (poloxamer)-(chondroitan sulfate)-(polyethylene glycol) polymer, polyethylene glycol, (hyaluronic acid)-(polygalacturonic acid) copolymer, (hyaluronic acid)-(gelatin)-(polyethylene glycol) polymer, (hyaluronic acid)-(collagen)-(sericin) polymer, (hyaluronic acid)-dextran copolymer, star polyethylene glycol, (star polyethylene glycol)-dextran copolymer, lysine-functionalized polyethylene glycol, (polyethylene glycol)-(dendritic lysine) polymer, polyethylene glycol-polylysine copolymer, thioloated gellan, acylated-sulfobetaine-starch, acrylated poloxamine, polyamidoamine dendrimer, (polyamidoamine dendrimer)-dextran copolymer, chitosan-dextran copolymer, chitosan-alginate copolymer, (carboxymethyl chitosan)-(carboxymethyl cellulose) copolymer, hyaluronic acid, tetra-succinimidyl substituted polyethylene glycol, tetra-thiol-substituted polyethylene glycol, and mixtures thereof,
xiv. an electro-functional polymer selected from a (polyethylene glycol)-(dendritic thioester) polymer, acrylated four-arm polymer containing (poly(p-phenylene oxide))-(polyethylene glycol)-(poly(p-phenylene oxide)), poly(methacrylamide-co-methacrylate)-gellan gum copolymer, chitosan-polylysine copolymer, hyaluronic acid, and mixtures thereof;
xv. a pH-sensitive polymer selected from (polyethylene glycol)-polyaspartylhydrazide copolymer, chitosan-alginate copolymer, chitosan-(gellan gum) copolymer, and mixtures thereof,
xvi. an ion-sensitive polymer selected from an alginate-chitosan-genipin polymer, chitosan-alginate copolymer, chitosan-(gellan gum) copolymer, gellan gum-kappa carrageenan copolymer, and mixtures thereof,
xvii. a photo-sensitive polymer selected from a (polyethylene glycol)-lactide, (polyethylene glycol)-fibrinogen polymer, acrylate-(polyethylene glycolyl)-acrylate, alginate, gelatin, pHEMA-co-APMA-polyamidoamine, poly(6-aminohexyl propylene phosphate), carboxymethyl chitan, hyaluronic acid, and mixtures thereof,
xviii. an enzyme-reactive polymer selected from a (polylysine)-(polyethylene glycol)-tyramine polymer, gelatin, pullulan, poly(phenylene oxide)-polyethylene glycol copolymer, gelatin-chitosan copolymer, and mixtures thereof;
xix. a pressure-sensitive polymer selected from (polyethylene glycol)-dihydroxyacetone;
xx. free-radical sensitive polymer selected from a betaine-containing polymer; and
xxi. a polymer selected from a (carboxymethylchitosan)-(oxidized alginate) copolymer, hyaluronic acid, (hyaluronic acid)-(crosslinked alginate) copolymer, (vinyl phosphonic acid)-acrylamide polymer, (poly(vinyl alcohol))-(carboxymethyl cellulose) copolymer, and mixtures thereof, and
xxii. mixtures thereof.

In certain embodiments, the biocompatible polymer is a thermosensitive polymer selected from a hydroxybutyl chitosan, carboxymethyl chitosan, chitosan-(D)-glucose phosphate, (chitosan)-(hydroxypropylmethyl cellulose)-(glycerin) polymer, chitosan-(beta-glycerophosphate)-hydroxyethyl cellulose polymer, (hyaluronic acid)-(hyperbranched polyethylene glycol) copolymer, poloxamer, (poloxamer)-(chondroitan sulfate)-(polyethylene glycol) polymer, (poly(lactic acid))-(poloxamer)-(poly(lactic acid) polymer, (polyethylene glycol)-polyalanine copolymer, (polyethylene gly col)-(poly caprolactone)-(polyethylene glycol) polymer, (polyethylene glycol)-(polyester urethane) copolymer, [poly(beta-benzyl L-aspartate)]-(polyethylene glycol)-[poly(beta-benzyl L-aspartate)], polycaprolactone-(polyethylene glycol)-polycaprolactone polymer, poly(lactic-co-glycolic acid)-(polyethylene glycol)-(poly(lactic-co-glycolic acid)), polymethacrylamide-polmethacrylate copolymer, poly(methacrylamide-co-methacrylate)-gellan gum copolymer, thiolated gellan, acrylated poloxamine, poly(N-isopropylacrylamide), poly(phosphazene), collagen-(poly(glycolic acid)) copolymer, (glycosaminoglycan)-(polypeptide) polymer, (ulvan)-(polyisopropylacrylamide) copolymer, a mixture of poloxamers, a mixture of hyaluronic acid and (polycaprolactone-(polyethylene glycol)-polycaprolactone), and mixtures thereof.

In certain embodiments, the biocompatible polymer is a nucleo-functional polymer selected from a N—O carboxymethyl chitosan, (poloxamer)-(chondroitan sulfate)-(polyethylene glycol) polymer, polyethylene glycol, (hyaluronic acid)-(polygalacturonic acid) copolymer, (hyaluronic acid)-(gelatin)-(polyethylene glycol) polymer, (hyaluronic acid)-(collagen)-(sericin) polymer, (hyaluronic acid)-dextran copolymer, star polyethylene glycol, (star polyethylene glycol)-dextran copolymer, lysine-functionalized polyethylene glycol, (polyethylene glycol)-(dendritic lysine) polymer, polyethylene glycol-polylysine copolymer, thioloated gellan, acylated-sulfobetaine-starch, acrylated poloxamine, polyamidoamine dendrimer, (polyamidoamine dendrimer)-dextran copolymer, chitosan-dextran copolymer, chitosan-alginate copolymer, (carboxymethyl chitosan)-(carboxymethyl cellulose) copolymer, hyaluronic acid, tetra-succinimidyl substituted polyethylene glycol, tetra-thiol-substituted polyethylene glycol, and mixtures thereof.

In certain embodiments, the biocompatible polymer is an electro-functional polymer selected from a (polyethylene glycol)-(dendritic thioester) polymer, acrylated four-arm polymer containing (poly(p-phenylene oxide))-(polyethylene glycol)-(poly(p-phenylene oxide)), poly(methacrylamide-co-methacrylate)-gellan gum copolymer, chitosan-polylysine copolymer, hyaluronic acid, and mixtures thereof.

In certain embodiments, the biocompatible polymer is a pH-sensitive polymer selected from (polyethylene glycol)-polyaspartylhydrazide copolymer, chitosan-alginate copolymer, chitosan-(gellan gum) copolymer, and mixtures thereof.

In certain embodiments, the biocompatible polymer is an ion-sensitive polymer selected from an alginate-chitosan-genipin polymer, chitosan-alginate copolymer, chitosan-(gellan gum) copolymer, gellan gum-kappa carrageenan copolymer, and mixtures thereof;

In certain embodiments, the biocompatible polymer is a photo-sensitive polymer selected from a (polyethylene glycol)-lactide, (polyethylene glycol)-fibrinogen polymer, acrylate-(polyethylene glycolyl)-acrylate, alginate, gelatin, pHEMA-co-APMA-polyamidoamine, poly(6-aminohexyl propylene phosphate), carboxymethyl chitan, hyaluronic acid, and mixtures thereof.

In certain embodiments, the biocompatible polymer is an enzyme-reactive polymer selected from a (polylysine)-(polyethylene glycol)-tyramine polymer, gelatin, pullulan, poly(phenylene oxide)-polyethylene glycol copolymer, gelatin-chitosan copolymer, and mixtures thereof.

In certain embodiments, the biocompatible polymer is a pressure-sensitive polymer selected from (polyethylene glycol)-dihydroxyacetone.

In certain embodiments, the biocompatible polymer is a free-radical sensitive polymer selected from a betaine-containing polymer.

In certain embodiments, the biocompatible polymer is a polymer selected from a (carboxymethylchitosan)-(oxidized alginate) copolymer, hyaluronic acid, (hyaluronic acid)-(crosslinked alginate) copolymer, (vinyl phosphonic acid)-acrylamide polymer, (poly(vinyl alcohol))-(carboxymethyl cellulose) copolymer, and mixtures thereof.

The biocompatible polymer may be further characterized according to its molecular weight, such as the weight-average molecular weight of the polymer. In certain embodiments, the biocompatible polymer has a weight-average molecular weight in the range of from about 500 g/mol to about 1,000,000 g/mol. In certain embodiments, the biocompatible polymer has a weight-average molecular weight in the range of from about 1,000 g/mol to about 500,000 g/mol. In certain embodiments, the biocompatible polymer has a weight-average molecular weight in the range of from about 1,000 g/mol to about 100,000 g/mol. In certain embodiments, the biocompatible polymer has a weight-average molecular weight in the range of from about 2,000 g/mol to about 75,000 g/mol. In certain embodiments, the biocompatible polymer has a weight-average molecular weight in the range of from about 10,000 g/mol to about 75,000 g/mol. In certain embodiments, the biocompatible polymer has a weight-average molecular weight in the range of from about 25,000 g/mol to about 75,000 g/mol. In certain embodiments, the biocompatible polymer has a weight-average molecular weight in the range of from about 40,000 g/mol to about 60,000 g/mol. In certain embodiments, the biocompatible polymer polymer has a weight-average molecular weight in the range of from about 1,000 g/mol to about 10,000 g/mol.

### Features of the Curing Agent

The therapeutic methods for forming a hydrogel can be characterized according to the presence and/or identity of a curing agent used to facilitate formation of the hydrogel. The identity of the curing agent is tailored to the identity of the biocompatible polymer, as different biocompatible polymers form a hydrogel in response to different stimuli.

### Curing Agent for Thermosensitive Polymers

When the biocompatible polymer is a thermosensitive polymer, a curing agent may be used, and said curing agent may be heat. In certain embodiments, heat is applied to increase the temperature of the biocompatible polymer to a temperature that is at least 3, 6, 9, 12, 15, 18, 21, or 25° C. above ambient temperature. In certain embodiments, heat is applied to increase the temperature of the biocompatible polymer to a temperature that is from about 3-6, 6-9, 9-12, 12-15, 15-18, 18-21, or 21-25° C. above ambient temperature.

### Curing Agent for Nucleo-functional Polymers

When the biocompatible polymer is a nucleo-functional polymer, a curing agent may be used, and said curing agent may be an electrophile. In certain embodiments, the curing agent is a compound containing at least two electrophilic groups. In certain embodiments, the curing agent is a compound containing at least two functional groups capable of reaction with the nucleo-functional polymer. In certain embodiments, the curing agent is a polymer containing at least two electrophilic groups. In certain embodiments, the curing agent is a polymer containing at least two functional groups capable of reaction with the nucleo-functional polymer.

In certain embodiments, the curing agent is polymer selected from a polyalkylene and polyheteroalkylene polymer each being substituted by at least one electrophilic group. In certain embodiments, the curing agent is a biocompatible polyheteroalkylene polymer substituted by at least one electrophilic group. In certain embodiments, the curing agent is a biocompatible poly(oxyalkylene) polymer substituted by at least one electrophilic group. In certain embodiments, the curing agent is a biocompatible poly(ethyleneglycol) polymer substituted by at least one electrophilic group.

In certain embodiments, the electrophilic group is an alpha-beta unsaturated ester, maleimidyl, or

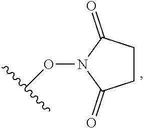 [SMILES: *ON1C(=O)CCC1=O]

each of which is optionally substituted by one or more occurrences of alkyl, aryl, or aralkyl. In certain embodiments, the electrophilic group is an alpha-beta unsaturated ester optionally substituted by one or more occurrences of alkyl, aryl, or aralkyl. In certain embodiments, the thiol-reactive group is —OC(O)CH═CH₂.

In certain embodiments, the curing agent has the formula:

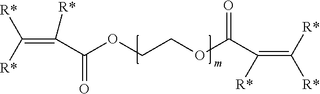

wherein R* is independently for each occurrence hydrogen, alkyl, aryl, or aralkyl; and m is an integer in the range of 5 to 15,000. In certain embodiments, R* is hydrogen. In yet other embodiments, m is an integer in the range of from about 20 to about 100, about 100 to about 500, about 500 to about 750, about 750 to about 1000, about 1000 to about 2000, about 2000 to about 5000, about 5000 to about 7500, about 7500 to about 10000, about 10000 to about 12500, about 12500 to about 15000.

The curing agent may be further characterized according to its molecular weight, such the weight-average molecular weight of the curing agent. Accordingly, in certain embodiments, the curing agent has a weight-average molecular weight in the range of from about 500 g/mol to about 1,000,000 g/mol. In certain embodiments, the curing agent has a weight-average molecular weight in the range of from about 1,000 g/mol to about 100,000 g/mol. In certain embodiments, the curing agent has a weight-average molecular weight in the range of from about 2,000 g/mol to about 8,000 g/mol. In certain embodiments, the curing agent has a weight-average molecular weight less than about 200,000 g/mol or less than about 100,000 g/mol.

In another more specific embodiment, the curing agent may be a poly(ethylene glycol) end-capped with at least two electrophilic groups capable of reaction with a nucleophile (e.g., where the electrophilic groups are thiol-reactive groups). The poly(ethylene glycol) may be linear, branched, a dendrimer, or multi-armed. The thiol-reactive group may be, for example, an acrylate, methacrylate, maleimidyl, haloacetyl, pyridyldithiol, or N-hydroxysuccinimidyl. An exemplary poly(ethylene glycol) end-capped with electrophilic groups may be represented by the formula Y—[—O—CH₂CH₂—]ₙ—O—Y wherein each Y is a thiol-reactive group, and n is, for example, in the range of 200 to 20,000. In another more specific embodiment, the curing agent may be CH₂=CHC(O)O—[—CH₂CH₂—O—]_{b}—C(O)CH═CH₂, wherein b is, for example, in the range of about 200 to about 20,000. Alternatively or additionally to the linear embodiments depicted above, the poly(ethylene glycol) may be a dendrimer. For example, the poly(ethylene glycol) may be a 4 to 32 hydroxyl dendron. In further embodiments, the poly(ethylene glycol) may be multi-armed. In such embodiments, the poly(ethylene glycol) may be, for example, a 4, 6 or 8 arm and hydroxy-terminated. The molecular weight of the poly(ethylene glycol) may be varied, and in some cases one of the thiol-reactive groups may be replaced with other structures to form dangling chains, rather than crosslinks. In certain embodiments, the molecular weight (Mw) is less than 20,000, including all values and ranges from 200 to 20,000, such as 200 to 1,000, 1,000 to 10,000, etc. In addition, the degree of functionality may be varied, meaning that the poly(ethylene glycol) may be mono-functional, di-functional or multi-functional.

### Curing Agent for Electro-functional Polymers

When the biocompatible polymer is an electro-functional polymer, a curing agent may be used, and said curing agent may be a nucleophile. In certain embodiments, the curing agent is a compound containing at least two nucleophilic groups. In certain embodiments, the curing agent is a polymer containing at least two functional groups capable of reaction with the electro-functional polymer. In certain embodiments, the curing agent is a polymer containing at least two nucleophilic groups. In certain embodiments, the curing agent is a polymer containing at least two functional groups capable of reaction with the electro-functional polymer. In certain embodiments, the curing agent is a polymer containing at least two nucleophilic groups independent selected from the group consisting of amino, hydroxyl, and sulfhydryl. In certain embodiments, the curing agent is a polymer containing at least two nucleophilic groups independent selected from the group consisting of amino and hydroxyl.

Curing Agent for pH-Sensitive Polymers

When the biocompatible polymer is a pH-sensitive polymer, a curing agent may be used, and said curing agent may be an acid or a base. In certain embodiments, the curing agent is a Bronsted acid. In certain embodiments, the curing agent is an organic carboxylic acid compound. In certain embodiments, the curing agent is a Bronsted base. In certain embodiments, the curing agent is an amine.

### Curing Agent for Ion-Sensitive Polymers

When the biocompatible polymer is an ion-sensitive polymer, a curing agent may be used, and said curing agent may be an ion. In certain embodiments, the curing agent is an cation. In certain embodiments, the curing agent is an anion. In certain embodiments, the curing agent is a salt compound. In certain embodiments, the curing agent is an alkali metal cation (e.g., a sodium or potassium cation) or an alkaline earth metal cation (e.g., a calcium or magnesium cation).

Curing Agent for Photo-Sensitive Polymers

When the biocompatible polymer is a photo-sensitive polymer, a curing agent may be used, and said curing agent may be light. In certain embodiments, the curing agent comprises visible light, ultra-violet light, or a mixture thereof. In certain embodiments, the curing agent is visible light. In certain embodiments, the curing agent is ultra-violet light.

### Curing Agent for Enzyme-Reactive Polymers

When the biocompatible polymer is an enzyme-reactive polymer, a curing agent may be used, and said curing agent may be an enzyme. In certain embodiments, the curing agent is horseradish peroxidase.

### Curing Agent for Pressure-Sensitive Polymers

When the biocompatible polymer is a pressure-sensitive polymer, a curing agent may be used, and said curing agent may be change in pressure. In certain embodiments, the curing agent is an agent that increases pressure experienced by the pressure-sensitive polymer.

### Curing Agent for Free-Radical Sensitive Polymer

When the biocompatible polymer is a free-radical sensitive polymer, a curing agent may be used, and said curing agent may be an agent that generates a free radical.

### Exemplary Combinations of Biocompatible Polymer and Curing Agents

Exemplary combinations of biocompatible polymers and curing agents that can be used to form hydrogels for use in the therapeutic methods and ocular formulations are provided in Tables 1-5 below.

TABLE 1
Biocompatible Polymer Containing Chitosan

| Biocompatible Polymer | Curing Technique to Form Hydrogel |
|---|---|
| hydroxybutyl chitosan | Heat |
| N—O carboxymethyl chitosan | Reaction with (hyaluronic acid - aldehyde) |
| alginate-chitosan-genipin polymer | Reaction with extracellular Ca+2 |
| gelatin-chitosan copolymer | Enzymatic cross-linking via horseradish |
| | peroxidase and H₂O₂ |
| carboxymethyl chitosan | Heat |
| (carboxymethylchitosan)-(oxidized | — |
| alginate) copolymer | |
| chitosan-dextran copolymer | Chemical cross-linking |
| chitosan-(D)-glucose phosphate | — |
| chitosan-polylysine copolymer | cross-inking via Michael addition |
| chitosan-(gellan gum) copolymer | pH and ion sensitive |
| chitosan-alginate copolymer | Schiff-base reaction |
| (chitosan)-(hydroxypropylmethyl | Heat |
| cellulose)-(glycerin) polymer | |
| chitosan-(beta glycerophosphate)- | Heat |
| hydroxyethyl cellulose polymer | |
| (carboxymethyl chitosan)- | Schiff-base reaction |
| (carboxymethyl cellulose) copolymer | |

TABLE 2
Biocompatible Polymer Containing Hyaluronic Acid

| Biocompatible Polymer | Curing Technique to Form Hydrogel |
|---|---|
| hyaluronic acid | — |
| | Thiol-disulfide cross-lining via oxidized |
| | glutathione |
| | Photo cross-linking with visible light |
| | Azide-Cyclooctyne cross-linking via click |
| | chemistry |
| | Adipic dihydrazide - aldehyde cross-linking |
| | Phenolic hydroxyl cross-linking via glucose |
| | oxidase and horseradish peroxidase |
| (hyaluronic acid)-(crosslinked alginate) | — |
| copolymer | |
| (hyaluronic acid)-(polygalacturonic acid) | Schiff-base reaction |
| copolymer | |
| (hyaluronic acid)-(gelatin)-(polyethylene | Thiol-acrylate cross-linking |
| glycol) polymer | |
| (hyaluronic acid)-(hyperbranched | Heat |
| polyethylene glycol) copolymer | |
| (hyaluronic acid)-(collagen)-(sericin) | Chemical cross-linked via amide amine |
| polymer | bonding |
| (hyaluronic acid)-dextran copolymer | Thiol-vinyl cross-inking |
| mixture hyaluronic acid and | Heat |
| (polycaprolactone-(polyethylene glycol- | |
| polycaprolactone) | |

TABLE 3
Biocompatible Polymer Containing Poloxamer

| Biocompatible Polymer | Curing Technique to Form Hydrogel |
|---|---|
| poloxamer | Heat |
| | Heat and/or photo cross-linking using UV light |
| mixture of poloxamers (e.g., mixture of | Heat |
| poloxamer F127, poloxamer F68, and | |
| poloxamer P123) | |
| (poloxamer)-(chondroitan sulfate)- | Heat and/or chemical cross-linking via click |
| (polyethylene glycol) polymer | reaction |
| (poly(lactic acid))-(poloxamer)-(poly(lactic | Heat |
| acid) polymer | |

TABLE 4
Biocompatible Polymer Containing Polyethylene Glycol

| Biocompatible Polymer | Curing Technique to Form Hydrogel |
|---|---|
| polyethylene glycol | Thiol-vinyl cross-inking via Michael addition |
| | Thiol-maleimide reaction |
| | Chemical cross-linking via bio-orthogonal Cu |
| | free click reaction |
| star polyethylene glycol | Schiff-base chemistry between the aldehydes |
| | and the amines |
| (star polyethylene glycol)-dextran copolymer | amine - aldehyde cross-linking |
| lysine-functionalized polyethylene glycol | nucleophilic substitution |
| (polyethylene glycol)-lactide | photo cross-linked using visible light |
| (polyethylene glycol)-(dendritic lysine) | nucleophilic substitution |
| polymer | |
| (polyethylene glycol)-(dendritic thioester) | thiol-thioester exchange (native chemical |
| polymer | ligation) |
| (polyethylene glycol)-fibrinogen polymer | photo cross-linking using UV light |
| (polyethylene glycol)-dihydroxyacetone | shear thinning physical cross-linked |
| (polyethylene glycol)-polyaspartylhydrazide | pH sensitive |
| copolymer | |
| (polyethylene glycol) - polyalanine | Heat |
| copolymer | |
| (polyethylene glycol)-(polycaprolactone)- | Heat |
| (polyethylene glycol) polymer | |
| (polyethylene glycol)-(polyester urethane) | Heat |
| copolymer | |
| [poly(beta-benzyl L-aspartate)]-(polyethylene | Heat |
| glycol)-[poly(beta-benzyl L-aspartate)] | |
| (polylysine)-(polyethylene glycol)-tyramine | Enzymatic cross-linking |
| polymer | |
| polycaprolactone-(polyethylene glycol)- | Heat |
| polycaprolactone polymer | |
| poly(phenylene oxide)-polyethylene glycol | cross-linking via horseradish peroxidase |
| copolymer | |
| acrylate-(polyethylene glycolyl)-acrylate | Photo cross-linking |
| polyethylene glycol- polylysine copolymer | Nucleophilic substitution |
| poly(lactic-co-glycolic acid)-(polyethylene | Heat |
| glycol)-(poly(lactic-co-glycolic acid)) | |
| acrylated four-arm polymer containing | chemical crosslinking with N- |
| (poly(p-phenylene oxide))-(polyethylene | hydroxysuccinimide (NHS) for reaction with |
| glycol)-(poly(p-phenylene oxide)) | tissue amines |
| tetra-succinimidyl and tetra-thiol-derivatized | Chemical cross-linking |
| polyethylene glycol | |

TABLE 5
Additional Biocompatible Polymers

| Material | Curing Technique to Form Hydrogel |
|---|---|
| alginate | photo cross-linked with visible light |
| gelatin | Enzymatic cross-linking |
| | Photo cross-linking |
| | Chemical cross-linking via enzymatic reaction |
| polymethacrylamide - polmethacrylate | Heat |
| copolymer | |
| poly(methacrylamide-co-methacrylate)-gellan | Heat and/or thiol cross-linking via oxidation |
| gum copolmer | |
| gellan gum - kappa carrageenan copolymer | Ion activated cross-linking |
| thiolated gellan | Heat and/or chemical cross-linking |
| 2-methacryloyloxyethyl phosphorylcholine | pH sensitive physical cross-linking |
| copolymer | |
| acylated-sulfobetaine-starch | Michael type click reaction |
| betaine compound | free radical via disulfide cross-linker |
| pHEMA-co-APMA- | photo cross-linked using UV light |
| polyamidoamine | |
| (vinyl phosphonic acid)-acrylamide polymer | — |
| poly(6-aminohexyl propylene phosphate) | Photo cross-linked by UV light |
| acrylated poloxamine | Heat and cross-linking via Michael addition |
| pullulan | Enzymatic cross-linking |
| (poly(vinyl alcohol))-(carboxymethyl | — |
| cellulose) copolymer | |
| poly(N-isopropylacrylamide) | Heat |
| poly(phosphazene) | Heat |
| polyamidoamine dendrimer | Chemical cross-linking by Michael's addition |
| carboxymethyl chitan | Photo cross-linking with UV light |
| collagen-(poly(glycolic acid)) copolymer | Heat |
| (polyamidoamine dendrimer)-dextran | cross-linking by Schiff-base reaction |
| copolymer | |
| (glycosaminoglycan)-(polypeptide) polymer | Heat |
| (ulvan)-(polyisopropylacrylamide) | Heat |
| copolymer | |

### Relative Amount of Biocompatible Polymer and Curing Agent

The therapeutic methods and compositions for forming a hydrogel can be characterized according to relative amount of biocompatible polymer and, when present, curing agent used. Accordingly, in certain embodiments, the mole ratio of (i) biocompatible polymer to (ii) curing agent (when the curing agent is a physical material that can be quantified) is in the range of 10:1 to 1:10. In certain embodiments, the mole ratio of (i) biocompatible polymer to (ii) curing agent (when the curing agent is a physical material that can be quantified) is in the range of 5:1 to 1:5. In certain embodiments, the mole ratio of (i) biocompatible polymer to (ii) curing agent (when the curing agent is a physical material that can be quantified) is in the range of 2:1 to 1:2.

### Administration Features of Biocompatible Polymer and Curing Agent

The method may be further characterized according to whether the biocompatible polymer and the curing agent, when present, are administered together as a single composition to the vitreous cavity of the eye of the subject, or alternatively the biocompatible polymer and the curing agent are administered separately to the vitreous cavity of the eye of the subject. In certain embodiments, the biocompatible polymer and the curing agent are administered together as a single composition to the vitreous cavity of the eye of the subject. The single composition may further comprise, for example, a liquid pharmaceutically acceptable carrier for administration to the eye of a subject.

In certain other embodiments, the biocompatible polymer and the curing agent are administered separately to the vitreous cavity of the eye of the subject. Even when administered separately, the biocompatible polymer may be administered as a liquid ocular formulation comprising a liquid pharmaceutically acceptable carrier for administration to the eye of a subject. This facilitates easy administration of the biocompatible polymer through surgical ports in the eye of the subject. Similarly, the curing agent, when it is a physical material, may be administered as a liquid ocular formulation comprising a liquid pharmaceutically acceptable carrier for administration to the eye of a subject. This facilitates easy administration of the curing agent through surgical ports in the eye of the subject.

A liquid formulation containing (i) a biocompatible polymer and/or the curing agent and (ii) a liquid pharmaceutically acceptable carrier for administration to the eye of a subject may be further characterized according to the viscosity of the formulation. In certain embodiments, the liquid formulation has a viscosity within 10%, 25%, 50%, 75%, 100%, 150%, 200%, or 300% of water. In certain other embodiments, the liquid formulation has a viscosity such that it can be administered through a needle having a gauge of less than or equal to 23 using a force of no more than 5N. In certain embodiments, the liquid formulation has a viscosity such that 1-2 mL of the liquid formulation can be administered within 3 minutes using a needle having a gauge of less than or equal to 23 using a force of no more than 5N.

In a more specific embodiment, a biocompatible polymer and/or the curing agent (when present) are provided in an aqueous pharmaceutical composition for administration to the eye. Such aqueous pharmaceutical compositions are desirably low viscosity liquids. In embodiments, the liquids exhibit a viscosity in the range of 0.004 Pa*s to 0.5 Pa*s, including all values and ranges therein, such as 0.010 Pa*s to 0.05 Pa*s.

Additional Step of Removing Vitreous Humor from the Eye

The provided methods may optionally further comprise the step of removing vitreous humor from the eye prior to administration of the nucleo-functional polymer and the electro-functional polymer.

### III. Injectable Ocular Pharmaceutical Compositions

Pharmaceutical compositions comprising (i) a nucleo-functional polymer and/or an electro-functional polymer and (ii) a pharmaceutically acceptable carrier for administration to the eye. Preferably, the pharmaceutical composition is a liquid pharmaceutical composition are also provided. The invention also provides pharmaceutical compositions comprising (a) a nucleo-functional polymer that is a biocompatible polyalkylene polymer substituted by (i) a plurality of —OH groups, (ii) a plurality of thio-functional groups —R¹—SH, (iii) at least one polyethylene glycolyl group, and (iv) optionally one or more —OC(O)—(C₁-C₆ alkyl) groups; R¹ is an ester-containing linker and (b) a pharmaceutically acceptable carrier for administration to the eye are also provided. Preferably, the pharmaceutical composition is a liquid pharmaceutical composition. The pharmaceutically acceptable carrier may be water or any other liquid suitable for administration to the eye of a subject.

Another aspect of the invention provides (a) a nucleo-functional polymer that is a biocompatible polyalkylene polymer substituted by (i) a plurality of —OH groups, (ii) a plurality of thio-functional groups —R¹—SH wherein R¹ is an ester-containing linker, and (iii) optionally one or more —OC(O)—(C₁-C₆ alkyl) groups; (b) a poly(ethylene glycol) polymer; and (c) an aqueous pharmaceutically acceptable carrier for administration to the eye of a subject. In certain embodiments, the formulation, further comprises an electro-functional polymer that is a biocompatible polymer containing at least one thiol-reactive group. Features recited in Section II above characterizing, for example, the nucleo-functional polymer, a poly(ethylene glycol) polymer, and the formulation are reiterated herein.

In certain embodiments, the invention provides pharmaceutical compositions comprising (i) a biocompatible polymer described herein and (ii) a pharmaceutically acceptable carrier for administration to the eye. Preferably, the pharmaceutical composition is a liquid pharmaceutical composition. The pharmaceutically acceptable carrier may be water or any other liquid suitable for administration to the eye of a subject.

The pharmaceutical composition is sterile and may optionally contain a preservative, antioxidant, and/or viscosity modifier. Exemplary viscosity modifiers include, for example, acacia, agar, alginic acid, bentonite, carbomers, carboxymethylcellulose calcium, carboxymethylcellulose sodium, carrageenan, ceratonia, cetostearyl alcohol, chitosan, colloidal silicon dioxide, cyclomethicone, ethylcellulose, gelatin, glycerin, glyceryl behenate, guar gum, hectorite, hydrogenated vegetable oil type I, hydroxyethyl cellulose, hydroxyethylmethyl cellulose, hydroxypropyl cellulose, hydroxypropyl starch, hypromellose, magnesium aluminum silicate, maltodextrin, methylcellulose, polydextrose, poly(ethylene glycol), poly(methylvinyl ether/maleic anhydride), polyvinyl acetate phthalate, polyvinyl alcohol, potassium chloride, povidone, propylene glycol alginate, saponite, sodium alginate, sodium chloride, stearyl alcohol, sucrose, sulfobutylether (3-cyclodextrin, tragacanth, xanthan gum, and derivatives and mixtures thereof. In some embodiments, the viscosity modifier is a bioadhesive or comprises a bioadhesive polymer.

In some embodiments, the concentration of the viscosity modifier in the pharmaceutical composition ranges from 0.1 to 20% by weight. In certain embodiments, the concentration of the viscosity modifier in the pharmaceutical composition ranges from 5 to 20% by weight. In certain embodiments, the concentration of the viscosity modifier in the pharmaceutical composition is less than 20%, less than 15%, less than 10%, less than 9%, less than 8%, less than 7%, less than 6%, less than 5%, less than 4%, less than 3%, less than 2%, less than 1.8%, less than 1.6%, less than 1.5%, less than 1.4%, less than 1.2%, less than 1%, less than 0.9%, less than 0.8%, less than 0.7%, less than 0.6%, less than 0.5%, less than 0.4%, less than 0.3%, less than 0.2%, or less than 0.1% by weight.

The pharmaceutical composition may be further characterized according to its viscosity. In certain embodiments, the viscosity of the pharmaceutical composition is less than 4000 cP, less than 2000 cP, less than 1000 cP, less than 800 cP, less than 600 cP, less than 500 cP, less than 400 cP, less than 200 cP, less than 100 cP, less than 80 cP, less than 60 cP, less than 50 cP, less than 40 cP, less than 20 cP, less than 10 cP, less than 8 cP, less than 6 cP, less than 5 cP, less than 4 cP, less than 3 cP, less than 2 cP, less than 1 cP. In some embodiments, the viscosity of the pharmaceutical composition is at least 4,000 cP, at least 2,000 cP, at least 1,000 cP, at least 800 cP, at least 600 cP, at least 500 cP, at least 400 cP, at least 200 cP, at least 100 cP, at least 80 cP, at least 60 cP, at least 50 cP, at least 40 cP, at least 20 cP, at least 10 cP, at least 8 cP, at least 6 cP, at least 5 cP, at least 4 cP, at least 3 cP, at least 2 cP, at least 1 cP. In certain embodiments, the viscosity of the pharmaceutical composition is about 4,000 cP, about 2,000 cP, about 1,000 cP, about 800 cP, about 600 cP, about 500 cP, about 400 cP, about 200 cP, about 100 cP, about 80 cP, about 60 cP, about 50 cP, about 40 cP, about 20 cP, about 10 cP, about 8 cP, about 6 cP, about 5 cP, about 4 cP, about 3 cP, about 2 cP, about 1 cP. In some embodiments, the viscosity of the viscosity of the pharmaceutical composition is between about 5 cP and 50 cP.

The pharmaceutical composition may be further characterized according to its pH. In certain embodiments, the pharmaceutical composition has a pH in the range of from about 5 to about 9, or about 6 to about 8. In certain embodiments, the pharmaceutical composition has a pH in the range of from about 6.5 to about 7.5. In certain embodiments, the pharmaceutical composition has a pH of about 7.

In certain embodiments, the pharmaceutical composition contains water, and the formulation has a pH in the range of about 7.1 to about 7.7. In certain embodiments, the pharmaceutical composition contains water, and the formulation has a pH in the range of about 7.1 to about 7.6, about 7.1 to about 7.5, about 7.1 to about 7.4, about 7.2 to about 7.6, about 7.2 to about 7.5, about 7.2 to about 7.4, about 7.2 to about 7.3, about 7.3 to about 7.7, about 7.3 to about 7.6, about 7.3 to about 7.5, about 7.3 to about 7.4, about 7.4 to about 7.7, about 7.4 to about 7.6, or about 7.4 to about 7.5. In certain embodiments, the pharmaceutical composition contains water, and the formulation has a pH in the range of about 7.3 to about 7.5. In certain embodiments, the pharmaceutical composition contains water, and the formulation has a pH of about 7.4.

The pharmaceutical composition may be further characterized according to osmolality and the presence and/or identity of salts. For example, in certain embodiments, the pharmaceutical composition has an osmolality in the range of about 280 mOsm/kg to about 315 mOsm/kg. In certain embodiments, the pharmaceutical composition has an osmolality in the range of about 280 mOsm/kg to about 300 mOsm/kg. In certain embodiments, the pharmaceutical composition has an osmolality in the range of about 285 mOsm/kg to about 295 mOsm/kg. In certain embodiments, the pharmaceutical composition has an osmolality of about 290 mOsm/kg. In certain embodiments, the pharmaceutical composition further comprises an alkali metal salt. In certain embodiments, the pharmaceutical composition further comprises an alkali metal halide salt, an alkaline earth metal halide salt, or a combination thereof. In certain embodiments, the pharmaceutical composition further comprises sodium chloride. In certain embodiments, the pharmaceutical composition further comprises sodium chloride, potassium chloride, calcium chloride, magnesium chloride, or a combination of two or more of the foregoing.

The pharmaceutical composition may be further characterized according to features of the nucleo-functional polymer described herein above.

### IV. Kits for Use in Medical Applications

Another aspect of the invention provides a kit for treating a disorder. The kit comprises: i) instructions for achieving one of the methods described herein (e.g., method for contacting retinal tissue in the eye of a subject with a hydrogel, methods for supporting retinal tissue, and methods for treating a subject with a retinal detachment); and ii) an nucleo-functional polymer described herein, an electro-functional polymer described herein, and/or formulation described herein. In certain embodiments, the kit comprises: i) instructions for achieving one of the methods described herein (e.g., method for contacting retinal tissue in the eye of a subject with a hydrogel, methods for supporting retinal tissue, and methods for treating a subject with a retinal detachment); and ii) a biocompatible polymer described herein and/or curing agent (when present as a material) described herein. In certain embodiments, one or more of the polymers described herein for forming a hydrogel may be supplied as a lyophilized formulation that may be reconstituted with a diluent prior to administration. In certain embodiments, the lyophilized formulation dissolves completely in the diluent in about 15 minutes or less at room temperature. In some embodiments, the lyophilized formulation has a shelf-life of at least 12 months. In certain embodiments, the volume of hydrogel-forming solution administered to the subject is sufficient to fill the cavity of the subject's eye. In some embodiments, the volume sufficient to fill the cavity of the eye is at least 6 mL. In certain embodiments, the volume sufficient to fill the cavity of the eye is less than 6 mL.

The description above describes multiple aspects and embodiments of the invention. The patent application specifically contemplates all combinations and permutations of the aspects and embodiments.

### Examples

The invention now being generally described, will be more readily understood by reference to the following examples, which are included merely for purposes of illustration of certain aspects and embodiments of the present invention, and are not intended to limit the invention.

### Example 1—Solubility Analysis of a Thiolated Poly(Vinyl Alcohol) Polymer, and Preparation of Exemplary Hydrogels

The ability of PEG 400 to reduce the amount of time required to dissolve a thiolated poly(vinyl alcohol) polymer in phosphate buffered saline was evaluated. Impact of PEG 400 on formation of a hydrogel from a phosphate buffered saline solution containing PEG 400, thiolated poly(vinyl alcohol) polymer, and a poly(ethylene glycol) diacrylate was evaluated. Experimental procedures and results are provided below.

### Part I—Experimental Procedures

Thiolated poly(vinyl alcohol) polymer having a weight-average molecular weight of approximately 31,000 g/mol was added to a solution of phosphate buffered saline that did or did not contain a poly(ethylene glycol) polymer having a number-average molecular weight of approximately 400 g/mol. The concentration of thiolated poly(vinyl alcohol) polymer in the phosphate buffered saline solution was approximately 8% w/v. The temperature of the solution of phosphate buffered saline was held at either room temperature (R.T.) or approximately 50° C., and monitored to determine the time required for all thiolated poly(vinyl alcohol) polymer to dissolve. Once all thiolated poly(vinyl alcohol) polymer had dissolved in the solution of phosphate buffered saline, samples were tested for time to crosslink with poly (ethylene glycol) diacrylate. the PVA solution was heated to 37° C., poly(ethylene glycol) diacrylate was added to the heated solution, and the time to crosslinking was measured. The poly(ethylene glycol) diacrylate had a weight-average molecular weight of approximately 3,400 g/mol. The concentration of poly(ethylene glycol) diacrylate in the heated solution was approximately 4% w/v.

The thiolated poly(vinyl alcohol) polymer is a poly(vinyl alcohol) polymer in which a portion of the hydroxyl groups on the polymer have been replaced with —OC(O)CH₂CH₂—SH. The thiolatedpoly(vinyl alcohol) polymer was prepared from poly(vinyl alcohol) based on procedures described in Ossipov et al. in Macromolecules (2008), vol. 41(11), pages 3971-3982.

### Part II—Results

Results of the experiment are provided in Table 6below.

TABLE 6

| | | Amount of | Time to | | Time to |
|---|---|---|---|---|---|
| | Thiolation | PEG 400 in | Dissolve | | Crosslink After |
| | Percentage | the PBS | Thiolated | Dissolution | Adding PEG- |
| | (on the PVA) | Solution | PVA | Temperature | Diacrylate |
| No. | (%) | (% w/v) | (min) | (° C.) | (min) |
| 1 | 5.625 | 0 | 25 | 50 | 2 |
| 2 | 5.625 | 5 | 11 | 50 | 2.8 |
| 3 | 5.275 | 0 | 47 | 50 | 2.3 |
| 4 | 5.275 | 5 | 19 | R.T. | 6 |
| 5 | 5.275 | 5 | 22 | R.T. | 7 |
| 6 | 6.125 | 5 | 8 | R.T. | 2.5 |
| 7 | 6.125 | 0 | 52 | R.T. | 2.5 |
| 8 | NA | 5 | 9 | R.T. | 3 |
| 9 | NA | 0 | 41 | R.T. | 2.5 |

NA means data not available.

### Example 2—Performance Specification for Exemplary Hydrogels

The following table provides various performance specifications for exemplary hydrogels formed by the methods, compositions, and formulations described herein.

TABLE 7

| | | Exemplary | |
|---|---|---|---|
| User Need | Specification | Requirement | |
| 1. May be provided as a | 1.1 Volume of hydrogel solution | ≥6 | ml |
| single-use kit with all | sufficient to fill one eye | | |
| necessary materials to | 1.1.1 Volume of thiolated-PVA after | ≥3 | ml |
| prepare and introduce in | reconstitution | | |
| a sterile manner | 1.1.2 Volume of PEG diacrylate | ≥3 | ml |
| sufficient hydrogel to | after reconstitution | | |
| tamponade one eye post | 1.2.1 Syringe volume sufficient for | 10 | ml min |
| vitrectomy. | injection of mixed solution | | |
| | 1.3 Amount of diluent provided is at | ≥6.5 | ml |
| | least 110% of volume required for the | | |
| | procedure | | |
| | 1.4 Accessory devices shall be sterile | SAL > 10-6 | |
| | 1.5 Diluent, t-PVA and PEGDA to be | Sterile | |
| | sterile | | |
| | 1.6 Cannula ID large enough to | ≥25 | Ga |
| | deliver hydrogel solution | | |
| | 1.7 Filter porosity small enough to | ≤5 | μm |
| | remove air bubbles | | |
| 2. The device is a safe | 2.1 Meets biocompatibility | Pass ISO 10993 | |
| and biocompatible | requirements for FDA | suite of tests | |
| | 2.1.1 Cytotoxicity | Non-cytotoxic | |
| | 2.1.2 Sensitization | Non-sensitizing | |
| | 2.1.3 Irritation | Non-irritant | |
| | 2.1.4 Acute Systemic Toxicity | Non-toxic | |
| | 2.1.5 Sub-acute Sytemnic Toxicity | Non-toxic | |
| | 2.1.6 Material Medicated | No pyrogenic | |
| | Pyrogenicity | components | |
| | 2.1.7 Implantation | NSD in tissue | |
| | | response compared | |
| | | to normal ocular | |
| | | tissue | |
| | 2.1.8 Genotoxicity | No clastogenic | |
| | | components | |
| | 2.2 No clinically significant increase | IOP < 35 mmHg | |
| | in Intra-ocular pressure | | |
| | 2.3 Endotoxin limit for injected | <0.2 | EU/mL |
| | hydrogel | | |
| | 2.4 Max Swelling | <50% | |
| | 2.5 pH of reconstituted hydrogel | 7.2-7.6 | |
| | solutions | | |
| | 2.6 Osmolality of reconstituted | 275-350 | mOsm/kg |
| | hydrogel solutions | | |
| | 2.7 Heat of reaction | <2° | C. |
| | 2.8 Degradation time | >7 days, <30 days | |
| | 2.9 Size of degradation components | <100 | kDa |
| | 2.10 Sterility | Sterile | |
| | 2.11 Particulates | ≤50 particles/mL > 10 um, | |
| | | ≤2 particles > 25 um | |
| | 2.12 Compatible with IOLs | No change in IOL | |
| | | Transparency | |
| | 2.13 Sub-retinal toxicity | Non-toxic | |
| 3. Provide tamponade to | 3.1 Volume of hydrogel solution | ≥6 | ml |
| the entire retinal | sufficient to fill one eye | | |
| surface. | 3.2 G′ Storage Modulus at full cure | >1000 | Pa |
| | 3.3 Swelling to ensure consistent | >5% & <20% | |
| | filling | within first 24 hours | |
| 4. Kit should have | 4.1 Storage conditions | RT (15-25 C.) stable | |
| similar storage or | 4.2 Shelf-life | ≥12 | months |
| handling conditions to a | 4.3 Shipping & Distribution | No change in | |
| non-gaseous intraocular | | properties | |
| fluid. | 4.4 Container Closure integrity | Meets ISO 8362 | |
| | 4.5 Package size | <50 | cu in |
| 5. Kit integrates into | 5.1 Time to prepare system | <15 | minutes |
| existing retinal surgery | 5.2 Crosslink time | 3 ± 2 minutes | |
| workflows | 5.3 Pot life of the polymer solutions | ≥30 | minutes |
| | 6.4 Force to inject | <5N | |
| 6. Kit components are | 6.1 Cannula compatibility | 25 | Ga max |
| compatible with | 6.2 Luer locks on accessories | Meet luer standards | |
| standard vitrectomy | | | |
| ports. | | | |
| 7. Preparation for | 7.1 Lyophilized polymers dissolve | Dissolves in 15 | |
| introduction into the eye | quickly without heating | minute or less @ | |
| should be easy. | | RT | |
| | 7.2 Easy to prepare | Prep nurse can | |
| | | prepare solutions | |
| | | for mixing | |
| 8. Hydrogel crosslinks | 8.1 Crosslink time | 3 ± 2 minutes | |
| quickly after injection | | | |
| into the eye. | | | |
| 9. Hydrogel | 9.1 Re-detachment rate | Non-inferior to | |
| demonstrates non- | | Standard of Care | |
| inferiority to standard of | 9.3 Visual acuity @ 7 days | Non-inferior to | |
| care. | | Standard of Care | |
| 10. Operator is able to | 10.1 Visible air-liquid interface | RI > 1.0 | |
| determine when | | | |
| sufficient hydrogel has | | | |
| been introduced into the | | | |
| eye. | | | |
| 11. Operators view of | 11.1 Transparency - absorbance in | <10% between 390 | |
| the retina remains | the visible spectrum | and 700 nm | |
| unobstructed upon | 11.2 Index of Refraction equal to | 1.32-1.34 | |
| completion of procedure | vitreous body | | |
| and post-operatively. | 11.3 Particulates | ≤50 particles/mL > 10 um, | |
| | | ≤2 particles > 25 um | |
| | 11.4 Entrapped air bubbles | <100/8 ml | |
| 12. Patients uncorrected | 12.1 Index of Refraction equal to | 1.32-1.34 | |
| visual acuity remains | vitreous | | |
| unaffected | 12.2 Transparency - absorbance in | <10% between 390 | |
| postoperatively and | the visible spectrum | and 700 nm | |
| throughout residence | 12.3 Particulates | ≤50 particles/mL > 10 um, | |
| time. | | ≤2 particles > 25 um | |
| | 12.4 Degrades without visible | ≤ 50 particles/mL > 10 um, | |
| | particulate formation | ≤2 particles > 25 um | |
| 13. Intraocular pressure | 13.1 IOP | <35 | mm Hg |
| remains clinically safe | 13.2 Swelling | <50% | |
| throughout residence | | | |
| time. | | | |
| 14. Hydrogel degrades | 14.1 Degradation Time | <30 | days |
| and diffuses from eye | 14.2 Size of degradation components | <100 | kDa |

| | | |
|---|---|---|
| and clear from the body | | Non-toxic |
| safely. | | degradation |
| | | components |
| 15. Patient has faster | 15.1 Habitual Corrected Visual | Within 3 lines of |
| uncorrected visual | Acuity | pre-operative acuity |
| recovery and is not | | in 75% patients at |
| required to he face | | one week |
| down post-operatively. | | |
| No post-operative | | |
| positioning or air-travel | | |
| restrictions should be | | |
| required. | | |
| 16. Removal is possible | 16.1 Crosslinked hydrogel can be | Standard vitrectomy |
| | broken up and aspirated | cutting and |
| | | aspiration devices |
| 17. Does not prevent | 17.1 Transparency - absorbance in | <10% between 400 |
| clinician from | the visible spectrum | and 600 nm |
| performing laser | | |
| retinopexy through the | | |
| Pykus Hydrogel | | |

### INCORPORATION BY REFERENCE

All of the references cited herein are hereby incorporated by reference in their entirety.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of contacting retinal tissue in an eye of a subject, the method comprising:
a. administering to the vitreous cavity of the eye of the subject an effective amount of (i) an electro-functional polymer, (ii) a nucleo-functional polymer, and (iii) a poly(ethylene glycol) polymer; and
b. allowing the nucleo-functional polymer and the electro-functional polymer to react to form a hydrogel in the vitreous cavity;
wherein the nucleo-functional polymer is a biocompatible polyalkylene polymer substituted by (i) a plurality of —OH groups, and (ii) a plurality of thio-functional groups —R¹—SH, wherein R¹ is an ester-containing linker; and
wherein the electro-functional polymer is a biocompatible polymer containing at least one thiol-reactive group.

2. The method of claim 1, wherein the subject has a physical discontinuity in the retinal tissue, a tear in the retinal tissue, a break in the retinal tissue, or a hole in the retinal tissue.

3. The method of claim 1 or 2, wherein the retinal tissue is contacted in a subject having undergone surgery for a macular hole, having undergone surgery to remove at least a portion of a epiretinal membrane, having undergone a vitrectomy for vitreomacular traction, having a rhegmatogenous retinal detachment, having tractional retinal detachment, or having serous retinal detachment.

4. The method of any one of claims 1-3, wherein the poly(ethylene glycol) polymer has a number-average molecular weight in the range of from about 200 g/mol to about 1,000 g/mol.

5. The method of any one of claims 1-4, wherein the nucleo-functional polymer is a biocompatible poly(vinyl alcohol) polymer substituted by a plurality of thio-functional groups —R¹—SH.

6. The method of claim 5, wherein the biocompatible poly(vinyl alcohol) polymer is a partially hydrolyzed poly(vinyl alcohol) polymer with a degree of hydrolysis of at least 85%.

7. The method of claim 5, wherein the biocompatible poly(vinyl alcohol) polymer is a fully hydrolyzed or substantially fully hydrolyzed poly(vinyl alcohol) polymer.

8. The method of any one of claims 1-7, wherein the thio-functional group —R¹—SH is —OC(O)—(CH₂CH₂)—SH.

9. The method of any one of claims 1-8, wherein the nucleo-functional polymer has a weight-average molecular weight up to about 75,000 g/mol.

10. The method of any one of claims 1-9, wherein the electro-functional polymer is a biocompatible polymer selected from a polyalkylene and polyheteroalkylene polymer, each being substituted by at least one thiol-reactive group.

11. The method of any one of claims 1-10, wherein the electro-functional polymer has a weight-average molecular weight up to about 15,000 g/mol.

12. The method of any one of claims 1-11, wherein the mole ratio of the (i) thio-functional 1 groups —R¹—SH to the (ii) thiol-reactive group is in the range of 10:1 to 1:10, 5:1 to 1:1, or 2:1 to 1:1.

13. The method of claim any one of claims 1-12, wherein the hydrogel has a refractive index greater than 1.0.

14. The method of claim any one of claims 1-13, wherein the hydrogel has a transparency of at least 95% for light in the visible spectrum when measured through hydrogel having a thickness of 2 cm.

15. The method of claim any one of claims 1-14, wherein the hydrogel has a gelation time of less than about 10 minutes after combining the nucleo-functional polymer and the electro-functional polymer or from about 1 minute to about 5 minutes after combining the nucleo-functional polymer and the electro-functional polymer.

16. The method of any of claims 1-15, wherein the hydrogel undergoes complete biodegradation from the eye of the subject within about 3 days to about 7 days, about 1 week to about 4 weeks, about 2 weeks to about 8 weeks, or about 4 months to about 6 months, or within 12 months or 24 months.

17. The method of any one of claims 1-16, wherein the hydrogel has a biodegradation half-life in the range of from about 1 week to about 3 weeks or from about 8 weeks to about 15 weeks when disposed within the vitreous cavity of the eye.

18. The method of any one of claims 1-17, wherein the hydrogel generates a pressure within the eye of less than about 35 mmHg or from about 20 mmHg to about 35 mmHg.

19. The method of any one of claims 1-18, wherein the electro-functional polymer, the nucleo-functional polymer, and the poly(ethylene glycol) polymer are each administered as separate liquid aqueous pharmaceutical compositions or together as a single, liquid aqueous pharmaceutical composition to the vitreous cavity of the eye of the subject.

20. The method of any one of claims 1-18, wherein the nucleo-functional polymer and the poly(ethylene glycol) polymer are administered together as a single, liquid aqueous pharmaceutical composition to the vitreous cavity of the eye of the subject.

21. The method of claim 19 or 20, wherein the separate pharmaceutical compositions or the single pharmaceutical composition comprises the poly(ethylene glycol) polymer in an amount of from about 0.5% w/v to about 30% w/v.

22. The method of any one of claims 19-21, wherein the separate pharmaceutical compositions or the single pharmaceutical composition comprises the nucleo-functional polymer in an amount of from about 0.5% w/v to about 15% w/v.

23. The method of any one of claims 19-22, wherein the separate pharmaceutical compositions or the single pharmaceutical composition comprises the electro-functional polymer in an amount of from about 0.5% w/v to about 15% w/v.

24. The method of claim any one of claims 19-23, wherein the separate pharmaceutical compositions or the single pharmaceutical composition has a pH in the range of about 7.2 to about 7.6 or has a pH of about 7.4.

25. The method of claim any one of claims 19-24, wherein the separate pharmaceutical compositions or the single pharmaceutical composition comprises phosphate buffered saline.

26. The method of claim any one of claims 19-25, wherein the separate pharmaceutical compositions or the single pharmaceutical composition has an osmolality in the range of about 275 mOsm/kg to about 350 mOsm/kg.

27. The method of any one of claims 1-26, wherein the poly(ethylene glycol) polymer is PEG 400 or PEGDA.

28. The method of any one of claims 1-27, wherein the nucleo-functional polymer is a biocompatible poly(vinyl alcohol) polymer substituted by a plurality of thio-functional groups —R¹—SH and having a thiolation percentage of up to about 30% or in a range of about 1% to about 10%, about 5% to about 10%, or about 5% to about 7%.

29. An injectable, pharmaceutical composition comprising:
a. a nucleo-functional polymer that is a biocompatible polyalkylene polymer substituted by (i) a plurality of —OH groups and (ii) a plurality of thio-functional groups —R¹—SH wherein R¹ is an ester-containing linker;
b. a poly(ethylene glycol) polymer; and
c. aqueous pharmaceutically acceptable carrier.

30. The composition of claim 29, further comprising an electro-functional polymer that is a biocompatible polymer containing at least one thiol-reactive group.

31. The composition of claim 29 or 30, wherein the composition comprises the poly(ethylene glycol) polymer in an amount of from about 0.5% w/v to about 30% w/v.

32. The composition of any one of claims 29-31, wherein the poly(ethylene glycol) polymer has a number-average molecular weight in the range of from about 200 g/mol to about 1,000 g/mol.

33. The composition of any one of claims 29-32, wherein the composition comprises the nucleo-functional polymer in an amount of from about 0.5% w/v to about 15% w/v.

34. The composition of any one of claims 30-33, wherein the composition comprises the electro-functional polymer in an amount of from about 0.5% w/v to about 15% w/v.

35. The composition of any one of claims 29-34, wherein the nucleo-functional polymer is a biocompatible poly(vinyl alcohol) polymer substituted by a plurality of thio-functional groups —R¹—SH.

36. The composition of any one of claims 29-35, wherein the nucleo-functional polymer is a biocompatible, partially hydrolyzed poly(vinyl alcohol) polymer with a degree of hydrolysis of at least 85%.

37. The composition of any one of claims 29-36, wherein the thio-functional group —R¹— SH is —OC(O)—(CH₂CH₂)—SH.

38. The composition of any one of claims 29-37, wherein the nucleo-functional polymer has a weight-average molecular up to about 75,000 g/mol.

39. The composition of any one of claims 30-38, wherein the electro-functional polymer is selected from a polyalkylene and polyheteroalkylene polymer each being substituted by at least one thiol-reactive group.

40. The composition of any one of claims 30-39, wherein the electro-functional polymer has a weight-average molecular weight up to about 15,000 g/mol.

41. The composition of any one of claims 29-40, wherein the poly(ethylene glycol) polymer is PEG 400 or PEGDA.

42. The composition of any one of claims 29-41, wherein the nucleo-functional polymer is a biocompatible poly(vinyl alcohol) polymer substituted by a plurality of thio-functional groups —R¹—SH and having a thiolation percentage of up to about 30% or in a range of about 1% to about 10%, about 5% to about 10%, or about 5% to about 7%.
